SEQUENCE LISTING (166 sequences)

SEQ ID NO: 1 - DNA, 44 bp, Artificial Sequence
  Primer HTJP-889
ctagaaagtataggaacttcgctagctctgctcgaggccatctg

SEQ ID NO: 2 - DNA, 34 bp, Artificial Sequence
  Primer HTJP-890
gttcgttccaatggccagcccgatgctatacttc

SEQ ID NO: 3 - DNA, 36 bp, Artificial Sequence
  Primer HTJP-891
agtatagcatcgggctggccattggaacgaactcgg

SEQ ID NO: 4 - DNA, 41 bp, Artificial Sequence
  Primer HTJP-892
gattgcgggacgatagcgtcaacatcgtagtccgacaaccg

SEQ ID NO: 5 - DNA, 41 bp, Artificial Sequence
  Primer HTJP-893
tcggactacgatgttgacgctatcgtcccgcaatccttcct

SEQ ID NO: 6 - DNA, 42 bp, Artificial Sequence
  Primer HTJP-894
ggtagagtaataacgcctaggacacgcaaaacgaggtacatt

SEQ ID NO: 7 - DNA, 29 bp, Artificial Sequence
  Primer HTJP-895
gtcctaggcgttattactctaccgcaagg

SEQ ID NO: 8 - DNA, 49 bp, Artificial Sequence
  Primer HTJP-896
taggaacttcaatcgatctagtcctaggctacgccaggaccgagcaagc

SEQ ID NO: 9 - DNA, 500 bp, Magnaporthe oryzae
tctgctcgaggccatctggcttttctctgctgtctgcctcgggaatgggatggaataccacgtacggtatttggcctccggtgccatccgaagcgagatgctttgagcttgaaaccccctcggcctgcacaggtgtctcatcgtgcatttaatccaacggcggcgagtcaaaacatcagctaattgaccaggtttctggattgtgaatgccaactttttgggtcttgaggagttgcggggtgggaaaaaagtaaagaaatttactgaggattttatcattgcgactataaaataaagcggcattgcaaatccttgcgttgctactatgtaaaatggactgtagttgtgctgctgaaaatagtttggcgattgtggattgtggattgtggattgtggattatggcaagttgtcaaggggcaagttgacgaaaatgattgtgtggtgtctgccagcaaattgagaacgtgggtatatatttcatcttttcatgattcccttc

SEQ ID NO: 10 - DNA, 91 bp, Aspergillus fumigatus
ggcttgcttgtcaagcaatggcatcattggtctagtggtagaattcgtcgttgccatcgacgaggcccgtgttcgattcacggatgatgca

SEQ ID NO: 11 - DNA, 78 bp, Artificial Sequence
  Cas9 sgRNA backbone
gttttagagctagaaatagcaagttaaaataaggctagtccgttatcaacttgaaaaagtggcaccgagtcggtgctt

SEQ ID NO: 12 - DNA, 215 bp, Magnaporthe oryzae
tttttttggctcttgggttcgaactgcccaaggcccatgttttggtcatctttttttttatgccccaccatttgggtcacccctgccaatcattccatctttgttcctacccttcacgtgtgctttccgaagccaaagttcccattcaacaactctccttgcgttttttttttcttgaagcttgtcacccgtcgatagtttctgccatttgcaat

SEQ ID NO: 13 - DNA, 886 bp, Aspergillus nidulans
cgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcgtcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcacc

SEQ ID NO: 14 - DNA, 4131 bp, Artificial Sequence
  dCas9_coding
atggacaagaagtatagcatcgggctggccattggaacgaactcggttggttgggctgtgattacggacgaatacaaggtgccatccaagaagtttaaggtcctgggaaacaccgaccgtcactcaatcaagaagaatctcattggagccctgctcttcgatagtggggagaccgccgaagctactcgactgaagcgaacggctcgccggcgttatacacgacgcaagaatcgcatctgctacctccaggagattttcagcaacgaaatggctaaggttgatgactcattctttcatcgactcgaagaaagtttcttggtcgaggaggataagaagcacgagcgccatccgatctttggtaacattgtggatgaggttgcctatcacgaaaagtacccaactatctatcatcttcgtaagaagctggtcgatagcacggacaaggctgatttgcgacttatctacctggcactcgcgcacatgattaagttccgcggccattttcttatcgagggtgacctgaaccccgataattctgacgttgataagctcttcatccagttggtccaaacctacaatcagctgtttgaggaaaaccctattaatgcatctggcgtggacgccaaggctatcctttcggcgcgcctgtctaagtcgcggcgtttggagaaccttatcgcacaactccccggcgaaaagaagaacggcctcttcggtaatttgattgcgttgtcacttggtctgactcctaacttcaagagtaattttgacctggcagaggatgcgaagctccagttgtctaaggatacgtatgatgacgatctcgacaacttgcttgcccaaatcggtgaccagtacgctgatcttttcctggccgctaagaatctctcagatgcaatcctgctcagtgacattttgcgggtcaacaccgagattactaaggcccccctgtcagctagtatgatcaagcggtatgatgagcaccatcaggacctcaccttgcttaaggccctcgtgcgtcagcaattgcctgagaagtacaaggaaatcttctttgaccaatccaagaacggatacgcagggtatattgatggcggtgcgagccaggaggaattctacaagtttatcaagccgattttggagaagatggacggcactgaggaactgctcgtcaagctgaatcgcgaagatttgcttcgtaagcaacgaacgttcgacaacggctccatcccgcaccagattcatctgggcgagctccacgccatccttcgacgccaggaagatttctacccatttctgaaggacaaccgtgagaagatcgaaaagattcttacattccgaatcccctactatgtgggacctttggcccgtgggaattcccgatttgcttggatgacccgaaagagcgaggaaaccatcactccgtggaacttcgaggaagtcgtggacaagggtgcatccgcgcagagcttcattgagcggatgaccaattttgataagaaccttccgaatgaaaaggtcctgccaaagcattcgctgctctacgagtatttcaccgtgtataacgaactgactaaggtcaagtacgtgacggagggaatgcggaagccagccttcctctcaggggaacaaaagaaggctatcgtcgatttgctttttaagaccaatcgtaaagtgactgttaagcagctgaaggaggattatttcaagaagattgaatgtttcgactccgtcgagatcagcggcgtggaagatcgctttaacgcttccctcggtacctaccacgacctgctcaagatcattaaggacaaggatttcctcgataacgaggaaaatgaggacatcttggaagatattgtcctcacgttgacactttttgaggaccgcgaaatgatcgaggaacggctcaagacatatgcccatttgttcgacgataaggtgatgaagcagctgaagcggcgtcgatacaccggatggggtcgccttagccggaagctgatcaacggcattcgagataagcaatctggtaagactatcttggatttccttaagtcggacggcttcgccaaccgcaattttatgcagcttattcacgacgattccctgacgttcaaggaggacatccagaaggcacaagtctcaggacaaggggattccctgcacgagcatatcgccaacctggctggatccccggcgatcaagaaggggattcttcagaccgtcaaggttgtcgacgagctggtcaaggtgatgggccgtcataagccagaaaacatcgtgattgagatggcccgagaaaatcagaccactcaaaagggtcagaagaacagccgcgagcggatgaagcggatcgaggaaggcattaaggaacttggttctcagatcctgaaggagcaccctgttgaaaacacacagctccaaaatgagaagctgtatctctactatttgcaaaatggacgcgacatgtacgtcgatcaggagctcgacattaaccggttgtcggactacgatgttgacgctatcgtcccgcaatccttccttaaggacgatagcattgataacaaggtgctgactcgctcagataagaaccggggcaagtccgacaatgttccaagcgaggaagtggttaagaagatgaagaactactggcgccaattgcttaatgccaagctcatcacacagcgcaagtttgacaacttgaccaaggccgagcggggagggctgagtgaactcgataaggctggcttcatcaagcgtcaactcgtggagacgcgacagatcacaaagcacgttgctcagattctggactcccggatgaacacaaagtacgacgagaatgataagctcatccgtgaagttaaggtcattaccctcaagtctaagttggtgtcggatttccgcaaggacttccaattttataaggttcgggagatcaacaattatcaccatgcacatgatgcgtacctcaacgcagtcgtgggaactgcgctcatcaagaagtatcccaagttggagtccgaattcgtctacggggattataaggtttacgacgtccgcaagatgatcgccaagagtgagcaggaaattggcaaggccacggctaagtatttcttttactccaacatcatgaatttctttaagacggagatcacactcgccaatggagaaatccgtaagcgacctttgattgagaccaacggcgagactggtgaaatcgtttgggataaggggcgcgacttcgctaccgtgcggaaggttctgagcatgccgcaagtcaatatcgtcaagaaaaccgaggtgcagacaggcggtttctctaaggaatcgattcttccaaagcgtaactctgacaagctgatcgctcgaaagaaggattgggaccccaagaagtatggagggttcgattctcctacagtggcatactcggttctcgttgtcgcgaaggttgagaagggaaagtctaagaagctgaagtcggtcaaggaactgctcgggatcaccattatggagcgctccagcttcgaaaagaatcccatcgactttctcgaggccaagggctataaggaagtcaagaaggatcttatcattaagctgcctaagtactctttgttcgagcttgaaaacggtcgaaagcgaatgctcgcatcggcaggagagttgcagaaggggaatgaattggcacttccctcaaagtacgtgaacttcctgtatctcgcgtcccactacgagaagctgaagggtagccctgaggacaacgaacagaagcaactttttgttgagcaacacaagcattatctggatgagatcattgaacagatttcagagttcagtaagcgcgtcatcctcgccgatgctaatctcgacaaggtgttgtcggcctacaacaagcaccgtgacaagccgatccgagagcaggctgaaaatatcattcatctgttcaccctcactaacttgggagcaccagcagcgttcaagtattttgatacgacaatcgaccgtaagcgatacacgtccacaaaggaggtgcttgatgcgaccctgattcatcaatccatcactgggctctatgaaacccgtatcgaccttagtcaactggggggcgaccctcccaagaagaagcgcaaggtctga

SEQ ID NO: 15 - protein, 1376 aa, Artificial Sequence
  dCas9_protein
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPPKKKRKV

SEQ ID NO: 16 - DNA, 1023 bp, Artificial Sequence
  hph selection marker
atgtcgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 17 - DNA, 21 bp, Artificial Sequence
  Primer IF_U6cas9_fwd
ttttctctgctgtctgcctcg

SEQ ID NO: 18 - DNA, 21 bp, Artificial Sequence
  Primer IF_dCasCDA_rev
gtcgccccccagttgactaag

SEQ ID NO: 19 - DNA, 26 bp, Artificial Sequence
  Primer IF_CDA3UTR_fwd
gcggacattcgatttatgccgttatg

SEQ ID NO: 20 - DNA, 24 bp, Artificial Sequence
  Primer IF_U63UTR_rev
agacagcagagaaaagccagatgg

SEQ ID NO: 21 - DNA, 20 bp, Artificial Sequence
  Primer CDAinsert_fwd
caactggggggcgacagcag

SEQ ID NO: 22 - DNA, 25 bp, Artificial Sequence
  Primer CDAinsert_rev
aaatcgaatgtccgcttatccggag

SEQ ID NO: 23 - DNA, 20 bp, Artificial Sequence
  Protospacer for pTNA197
cagcagtcctctgctctaga

SEQ ID NO: 24 - DNA, 20 bp, Artificial Sequence
  Protospacer for pTNA198
tccaacccactccctggaat

SEQ ID NO: 25 - DNA, 20 bp, Artificial Sequence
  Protospacer for pTNA199
ccagcatgttgactcggaat

SEQ ID NO: 26 - DNA, 20 bp, Artificial Sequence
  Protospacer for pTNA200
tgtcccagcatagtcgtcgt

SEQ ID NO: 27 - DNA, 60 bp, Artificial Sequence
  Primer wA_sense_1
ttcgattcacggatgatgcacagcagtcctctgctctagagttttagagctagaaatagc

SEQ ID NO: 28 - DNA, 60 bp, Artificial Sequence
  Primer wA_sense_1_rev
gctatttctagctctaaaactctagagcagaggactgctgtgcatcatccgtgaatcgaa

SEQ ID NO: 29 - DNA, 60 bp, Artificial Sequence
  Primer wA_sense_2
ttcgattcacggatgatgcatccaacccactccctggaatgttttagagctagaaatagc

SEQ ID NO: 30 - DNA, 60 bp, Artificial Sequence
  Primer wA_sense_2_rev
gctatttctagctctaaaacattccagggagtgggttggatgcatcatccgtgaatcgaa

SEQ ID NO: 31 - DNA, 60 bp, Artificial Sequence
  Primer wA_anti_1
ttcgattcacggatgatgcaccagcatgttgactcggaatgttttagagctagaaatagc

SEQ ID NO: 32 - DNA, 60 bp, Artificial Sequence
  Primer wA_anti_1_rev
gctatttctagctctaaaacattccgagtcaacatgctggtgcatcatccgtgaatcgaa

SEQ ID NO: 33 - DNA, 60 bp, Artificial Sequence
  Primer wA_anti_2
ttcgattcacggatgatgcatgtcccagcatagtcgtcgtgttttagagctagaaatagc

SEQ ID NO: 34 - DNA, 60 bp, Artificial Sequence
  Primer wA_anti_2_rev
gctatttctagctctaaaacacgacgactatgctgggacatgcatcatccgtgaatcgaa

SEQ ID NO: 35 - DNA, 5055 bp, Artificial Sequence
  dCas9-AID_coding
atggacaagaagtatagcatcgggctggccattggaacgaactcggttggttgggctgtgattacggacgaatacaaggtgccatccaagaagtttaaggtcctgggaaacaccgaccgtcactcaatcaagaagaatctcattggagccctgctcttcgatagtggggagaccgccgaagctactcgactgaagcgaacggctcgccggcgttatacacgacgcaagaatcgcatctgctacctccaggagattttcagcaacgaaatggctaaggttgatgactcattctttcatcgactcgaagaaagtttcttggtcgaggaggataagaagcacgagcgccatccgatctttggtaacattgtggatgaggttgcctatcacgaaaagtacccaactatctatcatcttcgtaagaagctggtcgatagcacggacaaggctgatttgcgacttatctacctggcactcgcgcacatgattaagttccgcggccattttcttatcgagggtgacctgaaccccgataattctgacgttgataagctcttcatccagttggtccaaacctacaatcagctgtttgaggaaaaccctattaatgcatctggcgtggacgccaaggctatcctttcggcgcgcctgtctaagtcgcggcgtttggagaaccttatcgcacaactccccggcgaaaagaagaacggcctcttcggtaatttgattgcgttgtcacttggtctgactcctaacttcaagagtaattttgacctggcagaggatgcgaagctccagttgtctaaggatacgtatgatgacgatctcgacaacttgcttgcccaaatcggtgaccagtacgctgatcttttcctggccgctaagaatctctcagatgcaatcctgctcagtgacattttgcgggtcaacaccgagattactaaggcccccctgtcagctagtatgatcaagcggtatgatgagcaccatcaggacctcaccttgcttaaggccctcgtgcgtcagcaattgcctgagaagtacaaggaaatcttctttgaccaatccaagaacggatacgcagggtatattgatggcggtgcgagccaggaggaattctacaagtttatcaagccgattttggagaagatggacggcactgaggaactgctcgtcaagctgaatcgcgaagatttgcttcgtaagcaacgaacgttcgacaacggctccatcccgcaccagattcatctgggcgagctccacgccatccttcgacgccaggaagatttctacccatttctgaaggacaaccgtgagaagatcgaaaagattcttacattccgaatcccctactatgtgggacctttggcccgtgggaattcccgatttgcttggatgacccgaaagagcgaggaaaccatcactccgtggaacttcgaggaagtcgtggacaagggtgcatccgcgcagagcttcattgagcggatgaccaattttgataagaaccttccgaatgaaaaggtcctgccaaagcattcgctgctctacgagtatttcaccgtgtataacgaactgactaaggtcaagtacgtgacggagggaatgcggaagccagccttcctctcaggggaacaaaagaaggctatcgtcgatttgctttttaagaccaatcgtaaagtgactgttaagcagctgaaggaggattatttcaagaagattgaatgtttcgactccgtcgagatcagcggcgtggaagatcgctttaacgcttccctcggtacctaccacgacctgctcaagatcattaaggacaaggatttcctcgataacgaggaaaatgaggacatcttggaagatattgtcctcacgttgacactttttgaggaccgcgaaatgatcgaggaacggctcaagacatatgcccatttgttcgacgataaggtgatgaagcagctgaagcggcgtcgatacaccggatggggtcgccttagccggaagctgatcaacggcattcgagataagcaatctggtaagactatcttggatttccttaagtcggacggcttcgccaaccgcaattttatgcagcttattcacgacgattccctgacgttcaaggaggacatccagaaggcacaagtctcaggacaaggggattccctgcacgagcatatcgccaacctggctggatccccggcgatcaagaaggggattcttcagaccgtcaaggttgtcgacgagctggtcaaggtgatgggccgtcataagccagaaaacatcgtgattgagatggcccgagaaaatcagaccactcaaaagggtcagaagaacagccgcgagcggatgaagcggatcgaggaaggcattaaggaacttggttctcagatcctgaaggagcaccctgttgaaaacacacagctccaaaatgagaagctgtatctctactatttgcaaaatggacgcgacatgtacgtcgatcaggagctcgacattaaccggttgtcggactacgatgttgacgctatcgtcccgcaatccttccttaaggacgatagcattgataacaaggtgctgactcgctcagataagaaccggggcaagtccgacaatgttccaagcgaggaagtggttaagaagatgaagaactactggcgccaattgcttaatgccaagctcatcacacagcgcaagtttgacaacttgaccaaggccgagcggggagggctgagtgaactcgataaggctggcttcatcaagcgtcaactcgtggagacgcgacagatcacaaagcacgttgctcagattctggactcccggatgaacacaaagtacgacgagaatgataagctcatccgtgaagttaaggtcattaccctcaagtctaagttggtgtcggatttccgcaaggacttccaattttataaggttcgggagatcaacaattatcaccatgcacatgatgcgtacctcaacgcagtcgtgggaactgcgctcatcaagaagtatcccaagttggagtccgaattcgtctacggggattataaggtttacgacgtccgcaagatgatcgccaagagtgagcaggaaattggcaaggccacggctaagtatttcttttactccaacatcatgaatttctttaagacggagatcacactcgccaatggagaaatccgtaagcgacctttgattgagaccaacggcgagactggtgaaatcgtttgggataaggggcgcgacttcgctaccgtgcggaaggttctgagcatgccgcaagtcaatatcgtcaagaaaaccgaggtgcagacaggcggtttctctaaggaatcgattcttccaaagcgtaactctgacaagctgatcgctcgaaagaaggattgggaccccaagaagtatggagggttcgattctcctacagtggcatactcggttctcgttgtcgcgaaggttgagaagggaaagtctaagaagctgaagtcggtcaaggaactgctcgggatcaccattatggagcgctccagcttcgaaaagaatcccatcgactttctcgaggccaagggctataaggaagtcaagaaggatcttatcattaagctgcctaagtactctttgttcgagcttgaaaacggtcgaaagcgaatgctcgcatcggcaggagagttgcagaaggggaatgaattggcacttccctcaaagtacgtgaacttcctgtatctcgcgtcccactacgagaagctgaagggtagccctgaggacaacgaacagaagcaactttttgttgagcaacacaagcattatctggatgagatcattgaacagatttcagagttcagtaagcgcgtcatcctcgccgatgctaatctcgacaaggtgttgtcggcctacaacaagcaccgtgacaagccgatccgagagcaggctgaaaatatcattcatctgttcaccctcactaacttgggagcaccagcagcgttcaagtattttgatacgacaatcgaccgtaagcgatacacgtccacaaaggaggtgcttgatgcgaccctgattcatcaatccatcactgggctctatgaaacccgtatcgaccttagtcaactggggggcgacagcagggctgaccccaagaagaagaggaaggtgggtggaggaggttctggaggtggaggttctgcagagtatgtgcgggccctctttgactttaatgggaatgatgaagaagaccttccctttaagaaaggagacatcctgagaatccgggataagcctgaagagcagtggtggaatgcagaggacagcgaaggaaagagggggatgattcctgtcccttacgtggagaagtattccggagactataaggaccacgacggagactacaaggatcatgatattgattacaaagacgatgacgataagtctaggatgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggcattcacgccgaaatctttagcattagaaaagtcgaagaatacctgcgcgacaaccccggacaattcacgataaattggtactcatcctggagtccttgtgcagattgcgctgaaaaaatcttagaatggtataaccaggagctgcgggggaacggccacactttgaaaatctgggcttgcaaactctattacgagaaaaatgcgaggaatcaaattgggctgtggaacctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgtttctagaggctccgga

SEQ ID NO: 36 - protein, 1685 aa, Artificial Sequence
  dCas9-AID_protein
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVGGGGSGGGGSAEYVRALFDFNGNDEEDLPFKKGDILRIRDKPEEQWWNAEDSEGKRGMIPVPYVEKYSGDYKDHDGDYKDHDIDYKDDDDKSRMTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAVSRGSG

SEQ ID NO: 37 - DNA, 6680 bp, Artificial Sequence
  PKS (wA) gene locus
atggagggtccatctcgtgtgtacctttttggagaccagaccagcgacatcgaagctggcctgcgccgtctgctccaagcgaagaatagtaccattgtccagtcctttttccagcaatgcttccatgcaattcgtcaagagatcgcgaagctcccgccgtctcatcggaagctcttcccacgcttcacgagcatcgttgatctcctttccaggagtcgtgaatcaggtcctagccctgtcctggagagtgcattgacatgcatctaccaattgggttgtttcattcagtaagtcaatgagttaccatctatacttgacaagtctgaccagccttcagcttttacggggatcttggacatgactaccctacaccctccaacagccatcttgttggcctgtgcactggtgttctgagctgcacggctgtaagttgcgccagaaatgttggagagcttattccagctgcagtggaatcggttgtaattgcactgcgactgggaatctgcgtttttcgagttcgagaactggtggactccgccgattccgagtcaacatgctggtcagcgttggtttctggaatcagtgaagcagaggctagccacctgatcgacgagtacagtagtaagaaggtgtgctcttccaactttaaacccccgcattgtgggatgctgacagatgcaggctactccgccttcttcgaaaccgtatatcagcgcggtaagctctaatggcgttactgtcagcgcaccacctacggtacttgatgaattcgtcgagacctgcatttccaagaattacaagccagtgaaggcccctattcatggcccgtaccatgcgccacatctgtatgatgataaggatatcgaccgcatcctgcagcagtcctctgctctagaaggactgaccggctgttcacccgttattcccatcatctccagtaacactggaaagccgatcaaggccaagtccatcaaagatctcttcaaggtcgcactggaggagatactcctacgacgactatgctgggacaaggtcacggagtcctgcacatcagtctgcaagaccggcacaaaccactcttgcaaattgtttccgatctcgagtagcgccactcaaagtttgttcacagtcctcaagaaggccggtgtgagcatcagcttggagactggggtaggagagatcgcgacgaacccagaaatgcggaaccttactggcaaggcagaaaattcaaagattgctatcattggtatgtctggaagatttcctgactcggatggtacggagagcttctggaacctcctgtacaaaggactcgacgtacatcgcaaagtccccgcagaccgttgggacgttgatgcccacgtcgacatgaccgggtcaaagagaaacacaagcaaagtggcttacggttgctggatcaacgaacccggcctgtttgacccccgattcttcaacatgtcgcctcgggaagcactccaagcagatcctgcacaacgtcttgcgttgcttacagcgtacgaggctctcgagatggctggcttcatcccggatagctctccatcgacgcagagggaccgtgtgggtattttctacggaatgaccagtgacgactaccgtgagatcaacagcggccaggacattgatacctatttcatccctggcggtaaccgagcatttacgccgggtcggataaactactacttcaaatttagcggccccagtgtgagcgttgacacagcgtgctcgtctagtcttgctgctatccacatggcttgcaattcgatctggagaaatgactgcgatgccgccatcactggaggtgtgaacattctgaccagccctgacaaccacgccggtctggatcggggccatttcctgtccaccactggcaactgtaacacctttgatgacggcgccgacggctactgtagagcggacggagttggaagcatcgttttgaagcggcttgaagatgccgaggccgacaacgacccgatcctggccgtcatcaacggtgcttacaccaaccactcggcggaggccgtgtcaatcactcgtccccatgttggcgcgcaagcattcatcttcaacaagctgctcaatgatgcgaatatcgaccctaaggacgtgagctacgtggaaatgcatggcactggaactcaagcaggtgatgcagtcgaaatgcagtccgttcttgacgtcttcgcaccagactaccgccggggtcccggtcaatcgcttcatatcggttctgccaaggcaaacattggacacggtgaatccgcatcaggagtgactgctcttgtcaaggtcctcctaatgatgagagagaacatgattcctcctcattgtggtatcaagaccaagatcaattccaatttcccgacagacttggcgaagcgcaatgttcatatcgccttccaacccactccctggaatcggccagcttcaggaaagcggcgaactttcgtcaacaacttttctgctgctggtggtaacactgctcttctactggaagatgctcccataccggaacgccaagggcaggaccccaggtcgttccatttggtctccgtgtcagcaagatcccagtctgcattgaagaacaacgtcgaagctctggtgaagtacattgactctcagggcaagtcctttggtgtgaaagagactgaattccttccaaacctggcgtacacgaccaccgcacgccgtatccaccatcccttccgtgtcattgcggttggagcgaacctacaatcactgcgtgactcgctgcatggtgctttgcaccgtgagacatataccccagttccctcaacggctcctggtattggtttcgtcttcaccggccaaggagcccaatactccggaatgggcaaggaactctaccgcagttgtttccaattccgaaccaccattgagcattttgactgcatcgcaagaagccagggccttccttctatccttcctcttgtcgatggaagcgtggctgtcgaagaacttagccctgtcgtggtacaagtgggaactacctgtgtacaaatggctctagtaaattactggactgctctgggtgtgaagccggcctttatcatcggacacagtcttggagactatgcagcccttaacacggccggtgttctatccaccagcgatacaatctatctttgtggccggcgtgctcagttgctgacgaaggaatgcaagattgggacacattcgatgctggccatcaaggcgtccctggcagaggtcaaacatttcctcagagacgagctccacgaagtctcttgtgttaacgcacctgcggagaccgtcgtcagcggccttgtcgctgatatcgacgagttggctcagaaatgctccacagagggtttgaagtcaaccaagctcaaggttccttacgcgttccattcctctcaggttgatcctatcttggaggccttcgaagatattgcccaaggtgtcaccttccacaagccgacaacacctttcgtctcagccctgttcggggaagtgatcaccgatgctaactgggagtgtctcggccccaagtacctgcgcgatcattgcagaaagacggtcaacttccttggcggcgtggaggctacgaggcatgcgaagctgaccaatgacaagactctgtgggttgagatcggctcacataccatttgctctggaatgatcaaagcaactcttggaccgcaagttacaacggttgcatctctacgccgcgaagaagatacctggaaggtcctttcgaacagtcttgcgagccttcatctggcgggtattgatatcaactggaagcaatatcaccaggactttagctcctctctccaggtcctccgcctcccagcctacaagtgggatctcaagaactactggattccctataccaacaacttctgcctgagcaagggcgctccagttgcgacagtagcggcagggccacagcatgagtacctgacaaccgcggctcagaaggtcattgagactcgaagtgatggagcaacagctacagtcgtgatagagaacgacattgctgatcccgagctcaaccgcgtcattcaaggccataaggtcaacggtactgctttgtgtccctcagtaagttaccgctcttgcccaacgactgcgttaagattcgtactaatcaggatatagtcactatatgccgacatctctcaaacgcttgcagagtatctcatcaaaaagtacaagcctgagtacgacggacttggactggatgtgtgtgaggtcacagtgccacgaccactgattgcgaaaggcggacagcagctctttagagtatctgcgacagcggattgggcggagaagaagacaacccttcagatatattcagtcactgcggaggggaagaagacggctgaccacgcaacttgcactgtccgattctttgactgcgctgctgcggaggcggaatggaaacgagtttcctaccttgtcaagaggagcattgaccgactgcatgatatcgccgaaaatggtgacgctcaccgtcttggtagaggcatggtttacaaactcttcgctgccttggttgattatgacgacaacttcaagtccattcgcgaggttattcttgacagtgaacagcacgaagcgactgcacgcgtcaagttccaagcaccacaaggcaatttccaccgaaacccgttctggattgacagttttggacacctgtctgggttcatcatgaacgcaagcgatgcaaccgactccaagaaccaggtctttgtcaatcacggatgggactccatgcgttgtttgaagaagttctcgcctgatgtcacctacaggacttatgttagaatgcagccttggaaagactccatctgggctggtgatgtctacgttttcgatggggatgatatcgttgcggtgtatggtgcagtcaaggtgagttcggcccgcgctcagttgcataagattcaaggtgctaatcattggtgtcacagttccaagccttatcacgcaagattctcgatacggtcctacctccagttggggcttcgaagggccccgccagaccagccgctagcgctcagaaggcggcccctgctgctgctgccagcaagagtcgtgctagcgccccggccccggcgaagcctgctgctaagcccagcgccccaagcttggtcaaacgggcacttaccatcctcgcagaggaagtgggtctgtctgaatccgagattacggatgatctggtcttcgcagactacggtgtggactcccttctttcgttgacggtcacgggcaggtatcgtgaagagctggatatcgatctcgaatcctccatcttcatcgaccagccgaccgtgaaagacttcaagcagttcttggccccaatgagccagggagaagccagcgatgggtccaccagtgacccagagtctagtagctccttcaatggtggctcttcaacagacgagtccagtgctgggtcccctgtcagctcaccaccaaatgagaaggttacgcaggtcgagcagcatgctacgataaaggagattcgcgccattttggccgatgagattggtgttacggaggaggagctgaaggacgatgagaacttgggagagatggggatggactctctgctttcgcttacggtgcttggtaggatccgtgagacattggatctggatctaccgggcgagttcttcatcgagaatcaaactctgaatgacgtggaggatgcattgggcctcaaacccaaggcagctcctgcgcctgcgcctgcgcctgctcccgtacccgcacccgtgtccgcgcccatattgaaggagcctgtccccaacgcaaactctaccatcatggcccgggcgagcccgcaccctcgatcaacctccattctgttgcaaggaaacccgaaaaccgcgaccaagaccctgttcctgttccctgatgggtctggctccgcaacatcgtatgcaaccattcccggagtgtccccggacgtgtgtgtctacggattgaactgcccgtacatgaagactccagagaagctcaagtatccccttgctgagatgacattcccctatctggccgagatccgccgcagacagcccaagggcccgtacaacttcggtggatggtctgcaggtggtatttgcgcctatgatgccgctcgctacctaatccttgaagagggcgaacaggttgaccgattgcttcttcttgactcgcccttccccattggcttagagaagttgcccactcggctgtacggcttcatcaactcaatgggtctctttggtgaaggcaacaaggctcccccggcctggttgctccctcatttcctggccttcattgattccctcgatacctacaaggccgtccccctcccctttgacgatccgaagtgggccaagaagatgccaaagacattcatggtctgggccaaggacggtatctgcagcaagccggatgacccgtggcccgagccggacccggacggcaagccggacacgagagagatggtctggctcctcaagaaccggaccgacatgggacccaacaagtgggacacactcgtcgggccccaaaacgtcggtggaatcactgtgatagagggtgcgaatcatttcaccatgactttgggacccaaggctaaagaattgggctcgttcattggcaacgccatggccaattaa

SEQ ID NO: 38 - DNA, 20 bp, Artificial Sequence
  Primer pks_seq_f2
tcatatcggttctgccaagg

SEQ ID NO: 39 - DNA, 20 bp, Artificial Sequence
  Primer pks_R
gttgttgacgaaagttcgcc

SEQ ID NO: 40 - DNA, 20 bp, Artificial Sequence
  Primer pks_F
actgcgactgggaatctgcg

SEQ ID NO: 41 - DNA, 23 bp, Artificial Sequence
  Primer pks_seq_r3
cttgtaattcttggaaatgcagg

SEQ ID NO: 42 - DNA, 31 bp, Artificial Sequence
  Primer BsrGI-3UTR-fwd
gtctaatgtacagcggacattcgatttatgc

SEQ ID NO: 43 - DNA, 32 bp, Artificial Sequence
  Primer NheI-FRT-rev
agcagagctagcgaagttcctatactttctag

SEQ ID NO: 44 - DNA, 30 bp, Artificial Sequence
  Primer Ex-UGItop-fwd
atgatctctagaggctccggaaccaacctg

SEQ ID NO: 45 - DNA, 25 bp, Artificial Sequence
  Primer UGIend-rev
aaatcgaatgtccgctgtacattag

SEQ ID NO: 46 - DNA, 5340 bp, Artificial Sequence
  dCas9-AID-UGI_coding
atggacaagaagtatagcatcgggctggccattggaacgaactcggttggttgggctgtgattacggacgaatacaaggtgccatccaagaagtttaaggtcctgggaaacaccgaccgtcactcaatcaagaagaatctcattggagccctgctcttcgatagtggggagaccgccgaagctactcgactgaagcgaacggctcgccggcgttatacacgacgcaagaatcgcatctgctacctccaggagattttcagcaacgaaatggctaaggttgatgactcattctttcatcgactcgaagaaagtttcttggtcgaggaggataagaagcacgagcgccatccgatctttggtaacattgtggatgaggttgcctatcacgaaaagtacccaactatctatcatcttcgtaagaagctggtcgatagcacggacaaggctgatttgcgacttatctacctggcactcgcgcacatgattaagttccgcggccattttcttatcgagggtgacctgaaccccgataattctgacgttgataagctcttcatccagttggtccaaacctacaatcagctgtttgaggaaaaccctattaatgcatctggcgtggacgccaaggctatcctttcggcgcgcctgtctaagtcgcggcgtttggagaaccttatcgcacaactccccggcgaaaagaagaacggcctcttcggtaatttgattgcgttgtcacttggtctgactcctaacttcaagagtaattttgacctggcagaggatgcgaagctccagttgtctaaggatacgtatgatgacgatctcgacaacttgcttgcccaaatcggtgaccagtacgctgatcttttcctggccgctaagaatctctcagatgcaatcctgctcagtgacattttgcgggtcaacaccgagattactaaggcccccctgtcagctagtatgatcaagcggtatgatgagcaccatcaggacctcaccttgcttaaggccctcgtgcgtcagcaattgcctgagaagtacaaggaaatcttctttgaccaatccaagaacggatacgcagggtatattgatggcggtgcgagccaggaggaattctacaagtttatcaagccgattttggagaagatggacggcactgaggaactgctcgtcaagctgaatcgcgaagatttgcttcgtaagcaacgaacgttcgacaacggctccatcccgcaccagattcatctgggcgagctccacgccatccttcgacgccaggaagatttctacccatttctgaaggacaaccgtgagaagatcgaaaagattcttacattccgaatcccctactatgtgggacctttggcccgtgggaattcccgatttgcttggatgacccgaaagagcgaggaaaccatcactccgtggaacttcgaggaagtcgtggacaagggtgcatccgcgcagagcttcattgagcggatgaccaattttgataagaaccttccgaatgaaaaggtcctgccaaagcattcgctgctctacgagtatttcaccgtgtataacgaactgactaaggtcaagtacgtgacggagggaatgcggaagccagccttcctctcaggggaacaaaagaaggctatcgtcgatttgctttttaagaccaatcgtaaagtgactgttaagcagctgaaggaggattatttcaagaagattgaatgtttcgactccgtcgagatcagcggcgtggaagatcgctttaacgcttccctcggtacctaccacgacctgctcaagatcattaaggacaaggatttcctcgataacgaggaaaatgaggacatcttggaagatattgtcctcacgttgacactttttgaggaccgcgaaatgatcgaggaacggctcaagacatatgcccatttgttcgacgataaggtgatgaagcagctgaagcggcgtcgatacaccggatggggtcgccttagccggaagctgatcaacggcattcgagataagcaatctggtaagactatcttggatttccttaagtcggacggcttcgccaaccgcaattttatgcagcttattcacgacgattccctgacgttcaaggaggacatccagaaggcacaagtctcaggacaaggggattccctgcacgagcatatcgccaacctggctggatccccggcgatcaagaaggggattcttcagaccgtcaaggttgtcgacgagctggtcaaggtgatgggccgtcataagccagaaaacatcgtgattgagatggcccgagaaaatcagaccactcaaaagggtcagaagaacagccgcgagcggatgaagcggatcgaggaaggcattaaggaacttggttctcagatcctgaaggagcaccctgttgaaaacacacagctccaaaatgagaagctgtatctctactatttgcaaaatggacgcgacatgtacgtcgatcaggagctcgacattaaccggttgtcggactacgatgttgacgctatcgtcccgcaatccttccttaaggacgatagcattgataacaaggtgctgactcgctcagataagaaccggggcaagtccgacaatgttccaagcgaggaagtggttaagaagatgaagaactactggcgccaattgcttaatgccaagctcatcacacagcgcaagtttgacaacttgaccaaggccgagcggggagggctgagtgaactcgataaggctggcttcatcaagcgtcaactcgtggagacgcgacagatcacaaagcacgttgctcagattctggactcccggatgaacacaaagtacgacgagaatgataagctcatccgtgaagttaaggtcattaccctcaagtctaagttggtgtcggatttccgcaaggacttccaattttataaggttcgggagatcaacaattatcaccatgcacatgatgcgtacctcaacgcagtcgtgggaactgcgctcatcaagaagtatcccaagttggagtccgaattcgtctacggggattataaggtttacgacgtccgcaagatgatcgccaagagtgagcaggaaattggcaaggccacggctaagtatttcttttactccaacatcatgaatttctttaagacggagatcacactcgccaatggagaaatccgtaagcgacctttgattgagaccaacggcgagactggtgaaatcgtttgggataaggggcgcgacttcgctaccgtgcggaaggttctgagcatgccgcaagtcaatatcgtcaagaaaaccgaggtgcagacaggcggtttctctaaggaatcgattcttccaaagcgtaactctgacaagctgatcgctcgaaagaaggattgggaccccaagaagtatggagggttcgattctcctacagtggcatactcggttctcgttgtcgcgaaggttgagaagggaaagtctaagaagctgaagtcggtcaaggaactgctcgggatcaccattatggagcgctccagcttcgaaaagaatcccatcgactttctcgaggccaagggctataaggaagtcaagaaggatcttatcattaagctgcctaagtactctttgttcgagcttgaaaacggtcgaaagcgaatgctcgcatcggcaggagagttgcagaaggggaatgaattggcacttccctcaaagtacgtgaacttcctgtatctcgcgtcccactacgagaagctgaagggtagccctgaggacaacgaacagaagcaactttttgttgagcaacacaagcattatctggatgagatcattgaacagatttcagagttcagtaagcgcgtcatcctcgccgatgctaatctcgacaaggtgttgtcggcctacaacaagcaccgtgacaagccgatccgagagcaggctgaaaatatcattcatctgttcaccctcactaacttgggagcaccagcagcgttcaagtattttgatacgacaatcgaccgtaagcgatacacgtccacaaaggaggtgcttgatgcgaccctgattcatcaatccatcactgggctctatgaaacccgtatcgaccttagtcaactggggggcgacagcagggctgaccccaagaagaagaggaaggtgggtggaggaggttctggaggtggaggttctgcagagtatgtgcgggccctctttgactttaatgggaatgatgaagaagaccttccctttaagaaaggagacatcctgagaatccgggataagcctgaagagcagtggtggaatgcagaggacagcgaaggaaagagggggatgattcctgtcccttacgtggagaagtattccggagactataaggaccacgacggagactacaaggatcatgatattgattacaaagacgatgacgataagtctaggatgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggcattcacgccgaaatctttagcattagaaaagtcgaagaatacctgcgcgacaaccccggacaattcacgataaattggtactcatcctggagtccttgtgcagattgcgctgaaaaaatcttagaatggtataaccaggagctgcgggggaacggccacactttgaaaatctgggcttgcaaactctattacgagaaaaatgcgaggaatcaaattgggctgtggaacctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgtttctagaggctccggaaccaacctgtccgacatcatcgagaaggagaccggcaagcagctcgttatccaggagtccatcctgatgctgcccgaggaggtcgaggaggtcatcggcaacaagcccgagtccgacatcctggtccacaccgcctacgacgagtccaccgacgagaacgtcatgctgctgacctccgacgcccccgagtacaagccctgggccctggtcatccaggactccaacggcgagaacaagatcaagatgctgtccggcggctcccccaagaagaagcgcaaggtctaa

SEQ ID NO: 47 - protein, 1779 aa, Artificial Sequence
  dCas9-AID-UGI_protein
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVGGGGSGGGGSAEYVRALFDFNGNDEEDLPFKKGDILRIRDKPEEQWWNAEDSEGKRGMIPVPYVEKYSGDYKDHDGDYKDHDIDYKDDDDKSRMTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAVSRGSGTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 48 - DNA, 20 bp, Artificial Sequence
  Primer pks_seq_r1
atttgcaagagtggtttgtg

SEQ ID NO: 49 - DNA, 3792 bp, Artificial Sequence
  Mad7_coding
atgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagactattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgttcatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgacaggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcaaattctcgagggacaagttgaagatttcgaacaaggattggttcgatttcatccagaacaagaggtacttgtaa

SEQ ID NO: 50 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 51 - protein, 192 aa, Artificial Sequence
  LbCpf1_partial_protein
IYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIEN

SEQ ID NO: 52 - protein, 189 aa, Artificial Sequence
  FnCpf1_partial_protein
IYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINN

SEQ ID NO: 53 - protein, 229 aa, Artificial Sequence
  Mad7_partial_protein
IYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGK

SEQ ID NO: 54 - DNA, 26 bp, Artificial Sequence
  Primer IF-Ptef-rev
ggtgaaggttgtgttatgttttgtgg

SEQ ID NO: 55 - DNA, 21 bp, Artificial Sequence
  Primer IF-nls-fwd
agcagggctgaccccaagaag

SEQ ID NO: 56 - DNA, 36 bp, Artificial Sequence
  Primer IF-PtfMd7-fwd
aacacaaccttcaccatgaacaacggcacaaacaac

SEQ ID NO: 57 - DNA, 36 bp, Artificial Sequence
  Primer IF-nlsMd7-rev
ggggtcagccctgctaggcaagtacctcttgttctg

SEQ ID NO: 58 - DNA, 88 bp, Artificial Sequence
  Primer sg-site_fwd
ttcgattcacggatgatgcagtcaaaagacctttttaatttctactcttgtagatagatcttttttttggctcttgggttcgaactgc

SEQ ID NO: 59 - DNA, 130 bp, Artificial Sequence
  Primer sg-site_rev
tttggcttcggaaagcacacgtgaagggtaggaacaaagatggaatgattggcaggggtgacccaaatggtggggcataaaaaaaaagatgaccaaaacatgggccttgggcagttcgaacccaagagcc

SEQ ID NO: 60 - DNA, 35 bp, Artificial Sequence
  Mad7 sgRNA backbone
gtcaaaagacctttttaatttctactcttgtagat

SEQ ID NO: 61 - DNA, 5028 bp, Artificial Sequence
  Mad7d-AID-UGI_coding
atgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagactattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgttcatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgcgaggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcaaattctcgagggacaagttgaagatttcgaacaaggattggttcgatttcatccagaacaagaggtacttgcctagcagggctgaccccaagaagaagaggaaggtgggtggaggaggttctggaggtggaggttctgcagagtatgtgcgggccctctttgactttaatgggaatgatgaagaagaccttccctttaagaaaggagacatcctgagaatccgggataagcctgaagagcagtggtggaatgcagaggacagcgaaggaaagagggggatgattcctgtcccttacgtggagaagtattccggagactataaggaccacgacggagactacaaggatcatgatattgattacaaagacgatgacgataagtctaggatgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggcattcacgccgaaatctttagcattagaaaagtcgaagaatacctgcgcgacaaccccggacaattcacgataaattggtactcatcctggagtccttgtgcagattgcgctgaaaaaatcttagaatggtataaccaggagctgcgggggaacggccacactttgaaaatctgggcttgcaaactctattacgagaaaaatgcgaggaatcaaattgggctgtggaacctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgtttctagaggctccggaaccaacctgtccgacatcatcgagaaggagaccggcaagcagctcgttatccaggagtccatcctgatgctgcccgaggaggtcgaggaggtcatcggcaacaagcccgagtccgacatcctggtccacaccgcctacgacgagtccaccgacgagaacgtcatgctgctgacctccgacgcccccgagtacaagccctgggccctggtcatccaggactccaacggcgagaacaagatcaagatgctgtccggcggctcccccaagaagaagcgcaaggtctaa

SEQ ID NO: 62 - protein, 1675 aa, Artificial Sequence
  Mad7d-AID-UGI_protein
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIARGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLPSRADPKKKRKVGGGGSGGGGSAEYVRALFDFNGNDEEDLPFKKGDILRIRDKPEEQWWNAEDSEGKRGMIPVPYVEKYSGDYKDHDGDYKDHDIDYKDDDDKSRMTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAVSRGSGTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 63 - DNA, 21 bp, Artificial Sequence
  Protospacer pTNA296 MdwA1
ttggagaccagaccagcgaca

SEQ ID NO: 64 - DNA, 21 bp, Artificial Sequence
  Protospacer pTNA297 MdwA2
gagaccagaccagcgacatcg

SEQ ID NO: 65 - DNA, 21 bp, Artificial Sequence
  Protospacer pTNA298 MdwA3
ttccagcaatgcttccatgca

SEQ ID NO: 66 - DNA, 21 bp, Artificial Sequence
  Protospacer pTNA299 MdwA4
cagcaatgcttccatgcaatt

SEQ ID NO: 67 - DNA, 21 bp, Artificial Sequence
  Protospacer pTNA300 MdwA5
catgcaattcgtcaagagatc

SEQ ID NO: 68 - DNA, 21 bp, Artificial Sequence
  Protospacer pTNA301 MdwA6
tcgagttcgagaactggtgga

SEQ ID NO: 69 - DNA, 21 bp, Artificial Sequence
  Protospacer pTNA302 MdwA7
cgatctcgagtagcgccactc

SEQ ID NO: 70 - DNA, 21 bp, Artificial Sequence
  Protospacer pTNA303 MdwA8
atccctggcggtaaccgagca

SEQ ID NO: 71 - DNA, 21 bp, Artificial Sequence
  Protospacer pTNA304 MdwA9
aggaaagcggcgaactttcgt

SEQ ID NO: 72 - DNA, 21 bp, Artificial Sequence
  Protospacer pTNA305 MdwA10
actgcatcgcaagaagccagg

SEQ ID NO: 73 - DNA, 21 bp, Artificial Sequence
  Protospacer pTNA306 MdwA11
gccctgtcgtggtacaagtgg

SEQ ID NO: 74 - DNA, 21 bp, Artificial Sequence
  Protospacer pTNA307 MdwA12
tggccggcgtgctcagttgct

SEQ ID NO: 75 - DNA, 21 bp, Artificial Sequence
  Protospacer pTNA324 MdwA13
tacaggaggttccagaagctc

SEQ ID NO: 76 - DNA, 21 bp, Artificial Sequence
  Protospacer pTNA325 MdwA14
tccagatcgaattgcaagcca

SEQ ID NO: 77 - DNA, 21 bp, Artificial Sequence
  Protospacer pTNA326 MdwA15
ctgaagctggccgattccagg

SEQ ID NO: 78 - DNA, 21 bp, Artificial Sequence
  Protospacer pTNA327 MdwA16
acacccagagcagtccagtaa

SEQ ID NO: 79 - DNA, 21 bp, Artificial Sequence
  Protospacer pTNA328 MdwA17
gggccgagacactcccagtta

SEQ ID NO: 80 - DNA, 21 bp, Artificial Sequence
  Protospacer pTNA329 MdwA18
agatcccacttgtaggctggg

SEQ ID NO: 81 - DNA, 21 bp, Artificial Sequence
  Protospacer pTNA330 MdwA19
ttctccgcccaatccgctgtc

SEQ ID NO: 82 - DNA, 61 bp, Artificial Sequence
  Primer 296-C9-sense
taatttctactcttgtagatttggagaccagaccagcgacatttttttggctcttgggttc

SEQ ID NO: 83 - DNA, 61 bp, Artificial Sequence
  Primer 296-C9-anti
gaacccaagagccaaaaaaatgtcgctggtctggtctccaaatctacaagagtagaaatta

SEQ ID NO: 84 - DNA, 61 bp, Artificial Sequence
  Primer 297-C6-sense
taatttctactcttgtagatgagaccagaccagcgacatcgtttttttggctcttgggttc

SEQ ID NO: 85 - DNA, 61 bp, Artificial Sequence
  Primer 297-C6-anti
gaacccaagagccaaaaaaacgatgtcgctggtctggtctcatctacaagagtagaaatta

SEQ ID NO: 86 - DNA, 61 bp, Artificial Sequence
  Primer 298-C4_7-sense
taatttctactcttgtagatttccagcaatgcttccatgcatttttttggctcttgggttc

SEQ ID NO: 87 - DNA, 61 bp, Artificial Sequence
  Primer 298-C4_7-anti
gaacccaagagccaaaaaaatgcatggaagcattgctggaaatctacaagagtagaaatta

SEQ ID NO: 88 - DNA, 61 bp, Artificial Sequence
  Primer 299-C1_4-sense
taatttctactcttgtagatcagcaatgcttccatgcaatttttttttggctcttgggttc

SEQ ID NO: 89 - DNA, 61 bp, Artificial Sequence
  Primer 299-C1_4-anti
gaacccaagagccaaaaaaaaattgcatggaagcattgctgatctacaagagtagaaatta

SEQ ID NO: 90 - DNA, 61 bp, Artificial Sequence
  Primer 300-C13-sense
taatttctactcttgtagatcatgcaattcgtcaagagatctttttttggctcttgggttc

SEQ ID NO: 91 - DNA, 61 bp, Artificial Sequence
  Primer 300-C13-anti
gaacccaagagccaaaaaaagatctcttgacgaattgcatgatctacaagagtagaaatta

SEQ ID NO: 92 - DNA, 61 bp, Artificial Sequence
  Primer 301-C2_8-sense
taatttctactcttgtagattcgagttcgagaactggtggatttttttggctcttgggttc

SEQ ID NO: 93 - DNA, 61 bp, Artificial Sequence
  Primer 301-C2_8-anti
gaacccaagagccaaaaaaatccaccagttctcgaactcgaatctacaagagtagaaatta

SEQ ID NO: 94 - DNA, 61 bp, Artificial Sequence
  Primer 302-C21-sense
taatttctactcttgtagatcgatctcgagtagcgccactctttttttggctcttgggttc

SEQ ID NO: 95 - DNA, 61 bp, Artificial Sequence
  Primer 302-C21-anti
gaacccaagagccaaaaaaagagtggcgctactcgagatcgatctacaagagtagaaatta

SEQ ID NO: 96 - DNA, 61 bp, Artificial Sequence
  Primer 303-C16-sense
taatttctactcttgtagatatccctggcggtaaccgagcatttttttggctcttgggttc

SEQ ID NO: 97 - DNA, 61 bp, Artificial Sequence
  Primer 303-C16-anti
gaacccaagagccaaaaaaatgctcggttaccgccagggatatctacaagagtagaaatta

SEQ ID NO: 98 - DNA, 61 bp, Artificial Sequence
  Primer 304-C11-sense
taatttctactcttgtagataggaaagcggcgaactttcgttttttttggctcttgggttc

SEQ ID NO: 99 - DNA, 61 bp, Artificial Sequence
  Primer 304-C11-anti
gaacccaagagccaaaaaaaacgaaagttcgccgctttcctatctacaagagtagaaatta

SEQ ID NO: 100 - DNA, 61 bp, Artificial Sequence
  Primer 305-C18-sense
taatttctactcttgtagatactgcatcgcaagaagccaggtttttttggctcttgggttc

SEQ ID NO: 101 - DNA, 61 bp, Artificial Sequence
  Primer 305-C18-anti
gaacccaagagccaaaaaaacctggcttcttgcgatgcagtatctacaagagtagaaatta

SEQ ID NO: 102 - DNA, 61 bp, Artificial Sequence
  Primer 306-C15-sense
taatttctactcttgtagatgccctgtcgtggtacaagtggtttttttggctcttgggttc

SEQ ID NO: 103 - DNA, 61 bp, Artificial Sequence
  Primer 306-C15-anti
gaacccaagagccaaaaaaaccacttgtaccacgacagggcatctacaagagtagaaatta

SEQ ID NO: 104 - DNA, 61 bp, Artificial Sequence
  Primer 307-C14-sense
taatttctactcttgtagattggccggcgtgctcagttgcttttttttggctcttgggttc

SEQ ID NO: 105 - DNA, 61 bp, Artificial Sequence
  Primer 307-C14-anti
gaacccaagagccaaaaaaaagcaactgagcacgccggccaatctacaagagtagaaatta

SEQ ID NO: 106 - DNA, 61 bp, Artificial Sequence
  Primer 324-C12,13-sense
taatttctactcttgtagattacaggaggttccagaagctctttttttggctcttgggttc

SEQ ID NO: 107 - DNA, 61 bp, Artificial Sequence
  Primer 324-C12,13-anti
gaacccaagagccaaaaaaagagcttctggaacctcctgtaatctacaagagtagaaatta

SEQ ID NO: 108 - DNA, 61 bp, Artificial Sequence
  Primer 325-C2,3-sense
taatttctactcttgtagattccagatcgaattgcaagccatttttttggctcttgggttc

SEQ ID NO: 109 - DNA, 61 bp, Artificial Sequence
  Primer 325-C2,3-anti
gaacccaagagccaaaaaaatggcttgcaattcgatctggaatctacaagagtagaaatta

SEQ ID NO: 110 - DNA, 61 bp, Artificial Sequence
  Primer 326-C17,18-sense
taatttctactcttgtagatctgaagctggccgattccaggtttttttggctcttgggttc

SEQ ID NO: 111 - DNA, 61 bp, Artificial Sequence
  Primer 326-C17,18-anti
gaacccaagagccaaaaaaacctggaatcggccagcttcagatctacaagagtagaaatta

SEQ ID NO: 112 - DNA, 61 bp, Artificial Sequence
  Primer 327-C15,16-sense
taatttctactcttgtagatacacccagagcagtccagtaatttttttggctcttgggttc

SEQ ID NO: 113 - DNA, 61 bp, Artificial Sequence
  Primer 327-C15,16-anti
gaacccaagagccaaaaaaattactggactgctctgggtgtatctacaagagtagaaatta

SEQ ID NO: 114 - DNA, 61 bp, Artificial Sequence
  Primer 328-C15,16-2-sense
taatttctactcttgtagatgggccgagacactcccagttatttttttggctcttgggttc

SEQ ID NO: 115 - DNA, 61 bp, Artificial Sequence
  Primer 328-C15,16-2-anti
gaacccaagagccaaaaaaataactgggagtgtctcggcccatctacaagagtagaaatta

SEQ ID NO: 116 - DNA, 61 bp, Artificial Sequence
  Primer 329-C6,7-sense
taatttctactcttgtagatagatcccacttgtaggctgggtttttttggctcttgggttc

SEQ ID NO: 117 - DNA, 61 bp, Artificial Sequence
  Primer 329-C6,7-anti
gaacccaagagccaaaaaaacccagcctacaagtgggatctatctacaagagtagaaatta

SEQ ID NO: 118 - DNA, 61 bp, Artificial Sequence
  Primer 330-C9,10-sense
taatttctactcttgtagatttctccgcccaatccgctgtctttttttggctcttgggttc

SEQ ID NO: 119 - DNA, 61 bp, Artificial Sequence
  Primer 330-C9,10-anti
gaacccaagagccaaaaaaagacagcggattgggcggagaaatctacaagagtagaaatta

SEQ ID NO: 120 - DNA, 21 bp, Artificial Sequence
  Primer pks_seq_f5
ttcttcaacatgtcgcctcgg

SEQ ID NO: 121 - DNA, 19 bp, Artificial Sequence
  Primer Primer pks_seq_r6
gtgttacagttgccagtgg

SEQ ID NO: 122 - DNA, 20 bp, Artificial Sequence
  Primer pks_seq_f4
ggtacttgatgaattcgtcg

SEQ ID NO: 123 - DNA, 22 bp, Artificial Sequence
  Primer MS-test-wA3
tgaattcaactctttacaatcg

SEQ ID NO: 124 - DNA, 3792 bp, Artificial Sequence
  Mad7d coding sequence
  CDS (1)..(3792)
atgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagagtattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgttcatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgcgaggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcaaattctcgagggacaagttgaagatttcgaacaaggattggttcgatttcatccagaacaagaggtacttgtaa

SEQ ID NO: 125 - protein, 1263 aa, Artificial Sequence
  Synthetic Construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKEYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIARGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 126 - protein, 1263 aa, Artificial Sequence
  Mad7d mature protein
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIARGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 127 - DNA, 624 bp, Artificial Sequence
  PmCDA1 coding sequence
atgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggcattcacgccgaaatctttagcattagaaaagtcgaagaatacctgcgcgacaaccccggacaattcacgataaattggtactcatcctggagtccttgtgcagattgcgctgaaaaaatcttagaatggtataaccaggagctgcgggggaacggccacactttgaaaatctgggcttgcaaactctattacgagaaaaatgcgaggaatcaaattgggctgtggaacctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgtt

SEQ ID NO: 128 - protein, 208 aa, Artificial Sequence
  PmCDA1 mature polypeptide
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 129 - DNA, 315 bp, Artificial Sequence
  Linker coding sequence
cctagcagggctgaccccaagaagaagaggaaggtgggtggaggaggttctggaggtggaggttctgcagagtatgtgcgggccctctttgactttaatgggaatgatgaagaagaccttccctttaagaaaggagacatcctgagaatccgggataagcctgaagagcagtggtggaatgcagaggacagcgaaggaaagagggggatgattcctgtcccttacgtggagaagtattccggagactataaggaccacgacggagactacaaggatcatgatattgattacaaagacgatgacgataagtctagg

SEQ ID NO: 130 - protein, 105 aa, Artificial Sequence
  Linker mature polypeptide
PSRADPKKKRKVGGGGSGGGGSAEYVRALFDFNGNDEEDLPFKKGDILRIRDKPEEQWWNAEDSEGKRGMIPVPYVEKYSGDYKDHDGDYKDHDIDYKDDDDKSR

SEQ ID NO: 131 - DNA, 297 bp, Artificial Sequence
  UGI coding sequence
tctagaggctccggaaccaacctgtccgacatcatcgagaaggagaccggcaagcagctcgttatccaggagtccatcctgatgctgcccgaggaggtcgaggaggtcatcggcaacaagcccgagtccgacatcctggtccacaccgcctacgacgagtccaccgacgagaacgtcatgctgctgacctccgacgcccccgagtacaagccctgggccctggtcatccaggactccaacggcgagaacaagatcaagatgctgtccggcggctcccccaagaagaagcgcaaggtc

SEQ ID NO: 132 - protein, 99 aa, Artificial Sequence
  UGI mature polypeptide
SRGSGTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 133 - DNA, 17429 bp, Artificial Sequence
  pAT3530
accaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcgctacataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttattcgactctatagtgaagttcctattctctagaaagtataggaacttctgaagtggggatttaaatgcggccgcgctgagggtttaatcgacgaagcagctgacggccagtgccaagcttaacgcgtacccgggcccagtatatgttccgcagatgactggagctctgccatacgtgccctctcaagcaccatttgttccatctacagagactagtcaccaactagtctatcaagactcacagggtacattgctgagaccaactgaccagaggcagggtagcggattgacggctccatctccttcacttacaaggtctattgaaagccctttagcatcaccaagcggagaatagattgttaagcttattttttgtatactgttttgtgatagcacgaagtttttccacggtatcttgtaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgccataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgactcccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggaccattgtgcaatgtaaactccatgccgtgggagttcttgtccttcaagtgcttgaccaatatgtttctgttggcagagggaacctgtcaactagttaataactagtcagaaactatgatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgttatcgcgaatgctcggcatactaactgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaagagatttcgactagttatgtacctggagtttggatataggaatgtgttgtggtaacgaaatgtaagggggaggaaagaaaaagtcgtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtaccaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaacggtgagagtccagtggtcgcagataactccctaaattccctgagctaactctaagtcgaccatgccgtttatggttagcgcctcccaaaaaggaatggccgacttagagttacctcttgacgactttttctttcctcccccttacatttcgttaccacaacacattcctatatccaaactccaggtacataactagtcgaaatctctttaaatctagtcaagaactagatacttaaccttcatactgcttagagatagcctttgaagctcttatttcgctctttgtctcactctcaccttcctcctcctcctccaatcctttttgctcgggactagtccaagactagtcaacagttagtatgccgagcattcgcgataacgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatcatagtttctgactagttattaactagttgacaggttccctctgccaacagaaacatattggtcaagcacttgaaggacaagaactcccacggcatggagtttacattgcacaatggtcctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggagtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatggcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttacaagataccgtggaaaaacttcgtgctatcacaaaacagtatacaaaaaataagcttaacaatctattctccgcttggtgatgctaaagggctttcaatagaccttgtaagtgaaggagatggagccgtcaatccgctaccctgcctctggtcagttggtctcagcaatgtaccctgtgagtcttgatagactagttggtgactagtctctgtagatggaacaaatggtgcttgagagggcacgtatggcagagctccagtcatctgcggaacatatactgggcccgggaagatctcatggtcatagctgtttccgttaattaatggttcacttctctttagaaatcaactgtgggttttgctttttgcttcattctctttgtcttctccatctttgatcaaatcctggactttctcaatccccagctaattcaatcatagtcagttttctatttttattatttctttttcttttgaaatgtgattaacaaccagtccgttatatatcttgtacccagattacgcccaactcgtgctcctcagccacaaagatactcaattgatagccaagatacatacataccacaaagtaaggactccatgcattgagtattactcatcgtattctagactactccaaaactcagcacatagacaaacaatacgaacctcgtctaggggtgattcagaggcggcaaagcggggttttcgcatttgatgttcctggcacttatgtaagcccacgcttcccgctcaactaaaccatcagccaatcagactgctcagatttatcttttgaagggtaaataaatcattgtaaagaagaacaagtggcttgcttgtcaagcaatggcatcattggtctagtggtagaattcgtcgttgccatcgacgaggcccgtgttcgattcacggatgatgcagtcaaaagacctttttaatttctactcttgtagatgcgatcgctttttttttgagcatttatcagcttgatatagaggtaggaatgtatggaggtgcagaatggctattttgttattggagcgggttcgaaacggagggcaggagactttttctaaatacgtcacgtgatatagagctgctttaattaacgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcgtcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacacaaccttcaccatgaacaacggcacaaacaacttccagaacttcattggaatctcgtcgttgcagaagactttgcgcaacgccctcatccccacagaaactacccagcagttcattgtgaagaacggaatcatcaaggaagatgaactccgaggcgagaaccgccagattttgaaggacatcatggatgattactaccgtggtttcatctcggaaacgctctcctccattgacgacatcgattggacttcgttgttcgaaaagatggaaatccagctcaaaaacggcgataacaaggataccttgatcaaggagcagaccgagtatcggaaggcgatccataagaagttcgccaacgatgatcggttcaagaacatgttctcggccaagttgatttccgacattctccccgaattcgtgatccataacaacaactactcggcgtcggagaaggaggagaagacgcaggtcatcaagttgttctcgaggttcgccacatcgttcaaagagtattttaagaatcgtgcgaactgtttctcggcagatgatatctcctcgtcctcctgtcaccgcattgtgaacgacaacgcggaaatcttcttctcgaacgcgttggtgtataggcgcatcgtgaagtccctctccaacgatgacatcaacaaaatctcgggagatatgaaggattcgctcaaggagatgtcgttggaggaaatctactcctatgagaagtatggcgagttcattacgcaggagggcatttccttctacaacgacatttgtggtaaagtcaactcgttcatgaacctctactgtcagaaaaacaaggagaacaaaaacctctataagctccagaagttgcataagcagatcctctgtatcgcagacacctcgtacgaggtcccttacaagttcgaatccgatgaggaggtctaccagtccgtcaacggattcttggacaacatctcctcgaaacacattgtcgagcggctccgaaagatcggcgataactacaacggctacaacttggacaaaatctatatcgtctccaagttctatgagtccgtctcgcagaaaacctatcgtgattgggagactatcaacactgcgctcgagattcactataacaacatcttgcctggtaacggcaaatcgaaagccgacaaggtgaagaaggccgtgaaaaacgatctccagaagtcgatcacagaaatcaacgaactcgtctcgaactacaagctctgttcggatgataacatcaaggcggaaacgtacatccatgaaatctcgcatatcttgaacaacttcgaggcccaggaactcaaatacaaccccgagatccacttggtcgagtcggagctcaaagcctcggagttgaagaacgtcttggatgtcatcatgaacgcattccactggtgttccgtgttcatgaccgaggaactcgtcgataaagacaacaacttctacgcggaactcgaggaaatctacgatgaaatctatcccgtgatctccctctacaacctcgtgcgaaactacgtcactcagaagccctattccaccaagaagatcaagctcaacttcggcatccccactctcgcagacggttggtcgaagtcgaaggagtactccaacaacgccattatcctcatgcgagacaacctctactacttgggtatcttcaacgcaaagaacaagccggataagaagatcattgaaggcaacacttcggaaaacaagggagactataagaagatgatctacaacctcctccctggacccaacaagatgattcctaaagtgttcctctcgtcgaagactggtgtggaaacgtataagccgtcggcctacatcttggagggctacaaacagaacaagcatatcaagtcctcgaaggacttcgacatcactttctgtcacgacctcatcgactatttcaagaactgtattgcaatccatccggaatggaagaacttcggcttcgatttctcggatacttcgacatacgaagatatctcgggattctaccgagaggtcgaattgcagggctataagattgattggacctacatctcggaaaaggatatcgacttgctccaggaaaagggccagctctacctcttccagatttacaacaaggacttctccaagaagtcgacgggtaacgacaacttgcacacaatgtatctcaaaaacctcttctcggaggagaacttgaaggatatcgtgctcaaattgaacggagaggccgaaatcttcttccgtaagtcctccatcaagaacccgatcatccataagaagggatcgatcttggtcaaccggacttacgaagcagaggaaaaagatcagttcggaaacatccagattgtcaggaagaacatccctgaaaacatctatcaggagttgtataagtacttcaacgacaagtcggataaggagctctccgacgaagcagccaaactcaagaacgtcgtcggacaccatgaagcagcaaccaacattgtgaaggactaccggtacacttacgacaagtacttcttgcacatgccgatcactatcaacttcaaagccaacaagaccggattcattaacgacaggatcctccagtacattgccaaagaaaaggacctccatgtcatcggtatcgcgaggggagaacggaacctcatctacgtctccgtgattgacacttgtggcaacattgtcgaacagaagtcgttcaacatcgtcaacggttacgattaccagattaagttgaaacagcaggaaggtgcgaggcagattgcgcgaaaggaatggaaggagattggcaaaatcaaggagattaaggaaggctacttgtcgttggtcatccacgaaatctcgaaaatggtgatcaaatacaacgccatcatcgccatggaagacctctcgtacggcttcaaaaagggacggttcaaagtggagcgtcaggtgtaccagaagttcgaaacaatgttgatcaacaagttgaactacttggtgttcaaggacatttccattaccgagaacggaggattgctcaagggttatcagctcacgtacatccccgacaagttgaaaaacgtgggacaccagtgtggctgtatcttctacgtgcctgcagcctacacgtcgaaaatcgaccctacaacaggattcgtgaacatcttcaagttcaaggatctcaccgtcgacgcgaagcgggagttcatcaaaaagttcgactccatccgctatgattcggagaagaacttgttctgtttcacattcgactacaacaacttcattactcagaacaccgtgatgtccaaatcgtcgtggtccgtgtacacgtatggtgtgcgcatcaaaaggcgcttcgtcaacggtcgcttctccaacgaatcggacacgatcgatatcacgaaagacatggagaaaacattggaaatgaccgacatcaactggcgtgacggccatgacctcaggcaggacatcatcgattacgagatcgtccagcacatcttcgaaatcttccgtctcaccgtgcagatgaggaactccctctccgagctcgaagatcgggattacgaccggctcatttcccctgtgttgaacgagaacaacatcttctacgactcggcaaaagcgggagatgcattgccgaaggacgccgatgcgaacggtgcatattgtattgcactcaagggtctctacgaaatcaagcagatcaccgaaaactggaaggaggacggcaaattctcgagggacaagttgaagatttcgaacaaggattggttcgatttcatccagaacaagaggtacttgcctagcagggctgaccccaagaagaagaggaaggtgggtggaggaggttctggaggtggaggttctgcagagtatgtgcgggccctctttgactttaatgggaatgatgaagaagaccttccctttaagaaaggagacatcctgagaatccgggataagcctgaagagcagtggtggaatgcagaggacagcgaaggaaagagggggatgattcctgtcccttacgtggagaagtattccggagactataaggaccacgacggagactacaaggatcatgatattgattacaaagacgatgacgataagtctaggatgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggcattcacgccgaaatctttagcattagaaaagtcgaagaatacctgcgcgacaaccccggacaattcacgataaattggtactcatcctggagtccttgtgcagattgcgctgaaaaaatcttagaatggtataaccaggagctgcgggggaacggccacactttgaaaatctgggcttgcaaactctattacgagaaaaatgcgaggaatcaaattgggctgtggaacctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgtttctagaggctccggaaccaacctgtccgacatcatcgagaaggagaccggcaagcagctcgttatccaggagtccatcctgatgctgcccgaggaggtcgaggaggtcatcggcaacaagcccgagtccgacatcctggtccacaccgcctacgacgagtccaccgacgagaacgtcatgctgctgacctccgacgcccccgagtacaagccctgggccctggtcatccaggactccaacggcgagaacaagatcaagatgctgtccggcggctcccccaagaagaagcgcaaggtctaatgtacagcggacattcgatttatgccgttatgacttccttaaaaaagcctttacgaatgaaagaaatggaattagacttgttatgtagttgattctacaatggattatgattcctgaacttcaaatccgctgttcattattaatctcagctcttcccgtaaagccaatgttgaaactattcgtaaatgtacctcgttttgcgtgtaccttgcttatcacgtgatattacatgacctggacagagttctgcgcgaaagtcataacgtaaatcccgggcggtaggtgcgtcccgggcggaaggtagttttctcgtccaccccaacgcgtttatcaacctcaactttcaacaaccatcatgccaccaaaagcgcgtaaaacaaagcgagatttgattgagcaagagggcaggatccaatgcgcgattcaagacattaaaaatggaaaatttcaaaaaattgcgcccgcagcgcgtgcatacaaaattcatcccaatactcctcgtgtactgtgtaagcgcccactaggtaatatgacatgattacgaattcgagctcggtacccggccggaactccacgtctagaggatccaccagtgattgaccaatgttttatcttctacagttctgcctgtctaccccattctagctgtacctgactacagagtagtttaattgtggttgaccccacagtcggaggcggaggaatacagcaccgatgtggcctgtctccatccagattggcacgcaatttttacacgcggaaaagatcgagatagagtacgactttaaatttagtccccggcggcttctattttagaatatttgagatttgattctcaagcaattgatttggttgggtcaccctcaattggataatatacctcattgctcggctacttcaactcatcaatcaccgtcataccccgcatataaccctccattcccacgatgtcgtccaagtcgcaattgacttacggtgctcgagccagcaagcaccccaatcctctggcaaagagactttttgagattgccgaagcaaagaagacaaacgttaccgtctctgctgatgtgacgacaacccgagaactcctggacctcgctgaccgtacggaagctgttggatccaatacatatgccgtctagcaatggactaatcaacttttgatgatacaggtctcggtccctacatcgccgtcatcaagacacacatcgacatcctcaccgatttcagcgtcgacactatcaatggcctgaatgtgctggctcaaaagtacaactttttgatcttcgaggaccgcaaattcatcgacatcggcaataccgtccagaagcaataccacggcggtgctctgaggatctccgaatgggcccacattatcaactgcagcgttctccctggcgagggcatcgtcgaggctctggcccagaccgcatctgcgcaagacttcccctatggtcctgagagaggactgttggtcctggcagagatgacccccaaaggatcgctggctacgggcgagtataccaaggcatcggttgactacgctcgcaaatacaagaacttcgttatgggtttcgtgtcgacgcgggccctgacggaagtgcagtcggatgtgtcttcagcctcggaggatgaagatttcgtggtcttcacgacgggtgtgaacctctcttccaaaggagataagcttggacagcaataccagactcctgcatcggctattggacgcggtgccgactttatcatcgccggtcgaggcatctacgctgctcccgacccggttgaagctgcacagcggtaccagaaagaaggctgggaagcttatatggccagagtatgcggcaagtcatgatttcctcttggagcaaaagtgtagtgccagtacgagtgttgtggaggaaggctgcatacattgtgcctgtcattaaacgatgagctcgtccgtattggcccctgtaatgccatgttttccgcccccaatcgtcaaggttttccctttgttagattcctaccagtcatctagcaaggcggccgcagctagcacaattgaggcatccccactaccgcattaagacctcagcgcggccgcaaatttaaataaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctttt

SEQ ID NO: 134 - DNA, 6651 bp, Aspergillus oryzae
atggaggggccacgcggcgtctatctcttcggagaccagacaagtgatttcgacgccggcttacgtcgcctcctacaagtaaagaataacacaattgttgcatcgttcttccagagatgctttcacgctttgcgccaagagatcgcgaggctttcaccatctgaacggaagatcttcccccggtttacgagcatagtggatctactggcgcgtcaccgggagtcagaccctaatccggctctggagagtgcgttgacctgtatctatcaattgggatgctttataaagtacgtgtaactgcagatcctgacccgtttgaacgagcctaacctgagatagctactacggagaccttggaaacgtgtacccatctgcttcagactgccatatagttggcctgtgcgcgggtcttcttagttctgcagctgtaagctgttcgaacaatgttggagaattgctccccgctgcggttgaagcggtggtggtagctctccgacttggtctatgcgtccttaaagttcgagagctggtgagctctgaccaagcgtcgtcaacaagctggtcagtcttgatttcagggattagcgagaaagatgcctcgcagcttataggagaattcactgctgaacgggtaagtcaattgatctgaaatagtttgcaggacagaatgttctaaccactggataaaggcaattcctccttcatccaaaccgtatatcagtgcggtgggatataacagtataaccatcagcgcaccgcctaaggtccttgatgatttaattgattctaggctgtctaagagccataagccggtgagggcgcaaatccatggtccttaccatgcagcacatctgtactatggccgagatgtcgacaggatcatcgaaagctgccataatgaggtcgtttcaaactacacaccccgtatccccgtactatcaagtactacgggacagccgatagaggccaaacacatgaaagatctacttaaggccgcccttgaagagattctactacgtcaactatgctgggagaaagtgaccgatgcctgctattccatattaaaaactgctcgtcatcaaccatgcaagttgttcccaatttcaagcactgcgacacaaagcttgtttacagctcttacgaaagccgggataaccgacatcgaagtggaaaatgggctcggagatgttcccacgaacccgaaggacaaccttaacatcagcggcagggcggactgctccaagatagctatcattggcatgtctggacgattcccagaagctgatggcacagagagtttctgggaccttctgtataatggcctcgatgtacaccggaaggtgcctgcagagcgttgggatgttgatgcccacgttgatcctaccggaacaaaacggaacaccagcaaggttccatacggatgctggataaacgaaccggggttatttgacccccgcttcttcaatatgtcgccacgcgaagccctccaggcagatcccgctcaaagacttgcattgctcacggcctatgaagctcttgaaatggccggctttatccccgacagcaccccttctacacagagggatcgagtcggcctcttctatggaatgactagcgatgactatcgggagataaatagtggtcaagatattgatacttactttatccctggtgggaatcgtgctttcacacctggccggataaactactatttcaagttcagtgggcccagcgtcagcgttgatacagcttgttcttcaagtcttgcggctattcatatggcttgcaattcgatctggagaaatgattgcgatgctgctattgctggaggtgtcaatatattgacaaaccctgataaccatgccggtcttgaccgtggccatttcctgtccagaaccgggaattgcaacacatttgacgatggtgctgatggctactgtagagcagatggagtgggtacaatcattctcaagcggctggaagacgctcaggcggacaacgatccaatcctcggtgtgatcaatggagcctataccaatcattcggcagaagcagtctcgattacccgccctcatgttggcgcacaagcgtttatctttaataagctattgaacgatgccaatatcgaccctaaggacgtcagctacgttgaaatgcatggaactggtactcaagctggggatgcggtggaaatgcaatcggtcttggatacgtttgctcccgactaccgccgtggaccaggacagtctctccatcttggttccgccaaagcaaatgttgggcatggagagtcagcatctggtgtaactgcacttgtgaaagtgctgctaatgatgaagaagaataccataccccctcattgtggtataaagactaagatcaaccacaacttccccacggatctcgcgcaacgaaatgtccacattgcctttcaacctaccccttggaacagaccggcttccggaaagcggcagtgcttcattaacaacttttcggcggctggtggaaataccgctcttttgatggaagacgctccaatcgctgaggttaaggggcaggacactcgacctgttcacgttgtgtctgtatcggcacgatcccagagtgcgctcaaaaacaacatcaactctctcgtaaaatacatcgacgaacaaggaaggtcattcaatgtgaacgaggcagactttatcccaagcttggcatacaccaccacagcacggcgtatccatcacccattccgtgtcacagctatcgggtctagtttgcaggagctgcgtgactcacttaacaacagctctcgtctggaaagctttacccctgtccctgcgacggcccctggcgtagggttcgtgttcgctggccaaggagctcagcacaccggaatgggaaggcaactatacgaaaaatgctctcaattccgggcaacaatgcagcacttcgattgcattagtcaaaaccaagggtttccttcgatccttcccttggttgacggaagcgtgcccgtggaggagctgggccctatcgtgacacagctcggcaccacatgtcttcagatggctttggtcaactattggggttcactaggtataaaacctgcgttcgttcttgggcatagtctcggggagtttgctgctttgaataccgcaggagtattatcgacttccgataccatctacctttgtggccgtcgggctaccctccttacagaatactgccaggttgggacacacgccatgctggctgtcaaggcttcctacccccaggtcaagcagttactgaaagaaggtgtggatgaagttgcctgtgtcaactcacccagtgagacagtcgtcagtggcctcaccgctgatattgatgacttggctcaaaggtgttccactgaaggttggaagtccactaaactaagggtaccgttcgctttccattctgcccaagttactccaattcttgaacggtttcaagaagaggcccagggtgtcacgttccgtaagccgtcgttaccgtttgtttcctcactccttggggaagtcatcaccgaatctaattacgatgtcctgggagctcaatatatggtgaagcagtgccggaagtcggtgaacttccttggtgctcttgaggccaccagatatgcgaaattgatgactgataagactgtctggctggaagttggtgcccataccatttgctctggtatgatcaaagcaacattcggtccccaggttaccactgtggcatctcttcgccgagaggagaatgcatggaaggtcctctccaatagtctatcggcccttcatttggctggcattgatattaattggaaagaatatcatcaagacttcagctccagccaccaggtgctcccacttccttcttacaagtgggatctcaagaactactggataccctacactaacaatttctgccttacgaagggtgctccccaaactgcaattcaagctgcaccacaaactacattcctgaccactgctgcgcaaaaggttgttgagagtcgcgacgacggtacaacagcgactgtcgtggtgcaaaatgacatcgctgatcctgagttgaaccgtgttatccaaggtcacaaggtcaatggagccgcactttgcccatcggtaagtattgcatgcattgccagactatcttgtgttataattcggctacttacgtattgcctagtcactctacgcagatattgcccagacacttggagagtatcttattgagaaatacaaacccgagttcaaagatcttggtctcgatgtgtgtgacatggtcgtaccgaagccactcatcgcgaagggaggagagcagctctttagagtctctgctattgctaattgggctgagaagaaggcttcagttcaagtatacgccgttaatgctgacggcaaaaagaccgtggatcatgcgtattgtacggtgaagttctttgataccaatgcctccgagctcgagtggaagagaatctcgtacctggtcaagagaagcatcgacagtcttcaccagaatgcggagacaggggaggctcaccgtatccagcgaggaatggtctataaacttttcagcgcgttggtcgattatgatgaaaatttcaagtcgattcgcgaggttatcctggacagcgacaataatgaggccaccgctcgtgtcaaattccaagcaccgccaggaaatttccaccgaaacccattctggattgacagtttcggtcacttgtccggattcattatgaatgcgagcgacgcgaccgactctaagaaccaagtatttgttaaccatggatgggattcgatgcgttgcctgaagaagttctcgcctgatgtcacttatcgcacttatgtgaggatgcagccatggcaaaacaacatttgggctggagatgtttatatctttgagggcgacgatattattgctgtcttcggaggtgtgaaggtgggtacctcactactgattttggttcctgcttactgacatgataattagttccaagcactggcacgcaagatacttgacactgttcttccccctgttggcggttcaaaggcaccaattacagcgaaatcaccacctccagctcgcactcagaaggccaacaccggcgccaagacccgtcctaaagcacctgttccttccaagtcgttcaccaaatcttctgggccgagtgttgtcgtacgcgcactcagcattctggcctcagaagttggcctggcagagtctgaaatctcagacgacatggtgtttgcggactacggtgtagactcactcctctcccttacagttactggcaggtatcgtgaagagttgaacctcgatttggactcctctgtgtttaccgatcatccaactgtcaacgacttcaagcggctcatcgcccaagtgagtccttcagagagccatgatggttcctccagtgaacaagagtcgaatttctctttcaacggtggcgagtcctcaagcgcaagcacacctgacataacgtcaccgccgaatgagaaggtagctcaagtcgagcaaaacggcaccatgaaggaaatccgtaacatcatggcggaggagatcggtgtacccgcagaagagatcgaccctgacgagaacttgggagagatgggtatggactcgcttctctcccttactgttcttggaagaatacgggagactttggacatggacctgccaggagagttcttcatcgaaaaccagaccctcaatgatatagaggtggctttggacctaaaacccaagactacctctgctccaattcctatgccagagccagtgaaattccctgaagctatccacgacctccagccaaagcttgctcaacatcccaaggccacatccatcctgttacaaggaaaccccaggacagcaacaaagacgttattcttgtttcctgacggctctggctcagctacatcttacgctaccatccccggactctctcctgacgtctgcgtttacgggttgaattgcccatatatgaagacacctgagaagctcaaatgcagcctagatgaactcactgcgccctatgtagcagagattcgtcgtcggcaacccaagggtccttacagcttcggtggctggtcagcaggagggatctgtgcatatgatgcggcacgccatctaatgtttgaggaaggtgaacaagtcgaccgcttgcttctccttgatacccccttccccatcggcctcgagaagctgccgcagagattgtacggcttcttcaactctatcggtctcttcggtgaaggtaaaacggcaccaccctcctggctcctaccccacttcctagcctttatcgacgctctcgacgcatacaaggccgcgccccttccattcaaagacgagaaatgggccaagaaactgcccaagacttatatcatctgggccaaggacggtgtttgcggtaagccgggagatccccggcctgatcccccgacagacggttccaaggatcccaaggagatggtctggcttcttaatgaccggaccgatctgggacctaacaagtgggatacattggttggacctgagaatattggtggaatcacagtaatggaagatgctaatcattttacgatgacgaagggcgaaaaagcgaaagagttgtctacatttatggctaacgccatggcttaa

SEQ ID NO: 135 - DNA, 20 bp, Artificial Sequence
  pAT3532 protospacer
acgctttgcgccaagagatc

SEQ ID NO: 136 - DNA, 20 bp, Artificial Sequence
  pAT3533 protospacer
cgccaagagatcgcgaggct

SEQ ID NO: 137 - DNA, 20 bp, Artificial Sequence
  pAT3534 protospacer
aagcactgcgacacaaagct

SEQ ID NO: 138 - DNA, 20 bp, Artificial Sequence
  pAT3535 protospacer
cattctgcccaagttactcc

SEQ ID NO: 139 - DNA, 20 bp, Artificial Sequence
  pAT3536 protospacer
cggaagccggtctgttccaa

SEQ ID NO: 140 - DNA, 20 bp, Artificial Sequence
  pAT3537 protospacer
tacctagtgaaccccaatag

SEQ ID NO: 141 - DNA, 62 bp, Artificial Sequence
  Primer oAT3941
aatttctactcttgtagatacgctttgcgccaagagatctttttttttgagcatttatcagc

SEQ ID NO: 142 - DNA, 62 bp, Artificial Sequence
  Primer oAT3942
aatttctactcttgtagatcgccaagagatcgcgaggcttttttttttgagcatttatcagc

SEQ ID NO: 143 - DNA, 62 bp, Artificial Sequence
  Primer oAT3943
aatttctactcttgtagataagcactgcgacacaaagcttttttttttgagcatttatcagc

SEQ ID NO: 144 - DNA, 62 bp, Artificial Sequence
  Primer oAT3944
aatttctactcttgtagatcattctgcccaagttactcctttttttttgagcatttatcagc

SEQ ID NO: 145 - DNA, 62 bp, Artificial Sequence
  Primer oAT3945
aatttctactcttgtagatcggaagccggtctgttccaatttttttttgagcatttatcagc

SEQ ID NO: 146 - DNA, 62 bp, Artificial Sequence
  Primer oAT3946
aatttctactcttgtagattacctagtgaaccccaatagtttttttttgagcatttatcagc

SEQ ID NO: 147 - DNA, 18 bp, Artificial Sequence
  Primer oAT3912
tccaagttctttgcatgc

SEQ ID NO: 148 - DNA, 18 bp, Artificial Sequence
  Primer oAT3613
tatctcaggttaggctcg

SEQ ID NO: 149 - DNA, 18 bp, Artificial Sequence
  Primer oJaL188
ccatggtccttaccatgc

SEQ ID NO: 150 - DNA, 22 bp, Artificial Sequence
  Primer oAT3616
tatttatctcccgatagtcatc

SEQ ID NO: 151 - DNA, 18 bp, Artificial Sequence
  Primer oAT919
ctggctgtcaaggcttcc

SEQ ID NO: 152 - DNA, 19 bp, Artificial Sequence
  Primer oAT1040
tttgtggtgcagcttgaat

SEQ ID NO: 153 - DNA, 17 bp, Artificial Sequence
  Primer oAT967
gcgaacacgaaccctac

SEQ ID NO: 154 - DNA, 17 bp, Artificial Sequence
  Primer oAT3618
tcaaagcagcaaactcc

SEQ ID NO: 155 - protein, 1232 aa, Sulfuricurvum sp. PC08-66
MLHAFTNQYQLSKTLRFGATLKEDEKKCKSHEELKGFVDISYENMKSSATIAESLNENELVKKCERCYSEIVKFHNAWEKIYYRTDQIAVYKDFYRQLSRKARFDAGKQNSQLITLASLCGMYQGAKLSRYITNYWKDNITRQKSFLKDFSQQLHQYTRALEKSDKAHTKPNLINFNKTFMVLANLVNEIVIPLSNGAISFPNISKLEDGEESHLIEFALNDYSQLSELIGELKDAIATNGGYTPFAKVTLNHYTAEQKPHVFKNDIDAKIRELKLIGLVETLKGKSSEQIEEYFSNLDKFSTYNDRNQSVIVRTQCFKYKPIPFLVKHQLAKYISEPNGWDEDAVAKVLDAVGAIRSPAHDYANNQEGFDLNHYPIKVAFDYAWEQLANSLYTTVTFPQEMCEKYLNSIYGCEVSKEPVFKFYADLLYIRKNLAVLEHKNNLPSNQEEFICKINNTFENIVLPYKISQFETYKKDILAWINDGHDHKKYTDAKQQLGFIRGGLKGRIKAEEVSQKDKYGKIKSYYENPYTKLTNEFKQISSTYGKTFAELRDKFKEKNEITKITHFGIIIEDKNRDRYLLASELKHEQINHVSTILNKLDKSSEFITYQVKSLTSKTLIKLIKNHTTKKGAISPYADFHTSKTGFNKNEIEKNWDNYKREQVLVEYVKDCLTDSTMAKNQNWAEFGWNFEKCNSYEDIEHEIDQKSYLLQSDTISKQSIASLVEGGCLLLPIINQDITSKERKDKNQFSKDWNHIFEGSKEFRLHPEFAVSYRTPIEGYPVQKRYGRLQFVCAFNAHIVPQNGEFINLKKQIENFNDEDVQKRNVTEFNKKVNHALSDKEYVVIGIDRGLKQLATLCVLDKRGKILGDFEIYKKEFVRAEKRSESHWEHTQAETRHILDLSNLRVETTIEGKKVLVDQSLTLVKKNRDTPDEEATEENKQKIKLKQLSYIRKLQHKMQTNEQDVLDLINNEPSDEEFKKRIEGLISSFGEGQKYADLPINTMREMISDLQGVIARGNNQTEKNKIIELDAADNLKQGIVANMIGIVNYIFAKYSYKAYISLEDLSRAYGGAKSGYDGRYLPSTSQDEDVDFKEQQNQMLAGLGTYQFFEMQLLKKLQKIQSDNTVLRFVPAFRSADNYRNILRLEETKYKSKPFGVVHFIDPKFTSKKCPVCSKTNVYRDKDDILVCKECGFRSDSQLKERENNIHYIHNGDDNGAYHIALKSVENLIQMK

SEQ ID NO: 156 - DNA, 3699 bp, Sulfuricurvum sp. PC08-66
  CDS (1)..(3699)
atgcttcacgctttcactaatcagtatcaactttctaaaacattgagattcggagcaactctgaaagaagacgagaaaaaatgcaagagtcatgaggaacttaaaggatttgtagatatttcatatgaaaacatgaaatcttccgctacaatcgctgaaagtttgaacgaaaatgaacttgtgaaaaaatgcgaaaggtgttattctgagatcgtgaaatttcataacgcttgggagaaaatctactacaggacagatcaaattgctgtctataaagatttctataggcaactgtcaagaaaagctagatttgatgccggtaagcaaaattcacaactgataaccttagcttccctttgcggtatgtaccaaggagctaagttaagtagatacataaccaattattggaaagataacattactaggcagaaatcatttcttaaagatttttcccaacagttacatcaatacactcgtgcactggaaaagtctgataaggctcatacaaaacctaatctgatcaacttcaataagacctttatggtgttggccaatctcgtgaacgaaatagttattcctctttctaatggagccatctcttttccaaacatctctaagctggaggacggggaagagtcccatcttatagaatttgcactcaatgactattctcagttgtctgaattaattggtgaattgaaggatgcaatagccactaacggtggttacacaccatttgcaaaggtgacccttaatcattatacagcagaacagaaaccacacgtatttaaaaatgatattgatgctaaaatacgtgagcttaagttgattgggttggttgagaccttgaaaggaaaatccagtgaacagattgaggaatacttctcaaatttagacaagtttagcacatacaacgataggaaccaatcagtaatcgtaagaactcaatgctttaagtataaacccattccttttttggttaagcatcaacttgcaaagtacatttcagaaccaaacggttgggatgaagacgccgtagctaaggttctggatgctgttggagctattcgttctccagcacatgattacgctaataaccaagaggggtttgatttaaaccattatcctattaaagtcgctttcgattatgcttgggagcagttggctaattctttgtataccaccgtgacttttccccaagaaatgtgcgaaaaatatttaaatagtatctacggttgtgaagtctccaaggagcctgtatttaaattctatgctgatctgctttatatcaggaagaatctggctgtactcgaacataagaacaatctgcccagtaatcaggaagagttcatatgtaagatcaacaacacatttgagaacatcgtgttaccatataagatttctcaatttgaaacttataagaaggatatacttgcctggataaacgatgggcatgaccataaaaaatatactgatgcaaaacagcaattaggttttattaggggtggactcaagggtaggattaaggcagaagaagtgtcccagaaagacaaatatggaaaaatcaagtcttattatgagaacccttacactaaactcaccaacgaatttaagcaaatatcctctacttatgggaagaccttcgctgagttaagagacaaatttaaagagaagaatgagatcaccaaaattacccacttcggtattataatagaagataaaaacagagacagatatttacttgcaagcgagttgaagcacgaacaaatcaaccacgtcagtactatccttaacaagttagataaatcatctgaatttattacctatcaagttaagagccttacaagcaaaacattgattaaattgattaaaaatcacaccacaaagaagggagccatttcaccatatgctgattttcacaccagtaaaaccggattcaacaagaatgaaatcgaaaagaattgggataattataagagagaacaggtattggttgagtatgtcaaagattgtctgaccgatagtactatggcaaaaaaccagaactgggcagagttcggttggaattttgagaaatgcaactcctatgaggatatcgaacacgaaatcgaccaaaaatcatatttgctgcagagcgatacaattagcaagcagagtattgcttccctcgtggaggggggctgtcttctccttcctataattaaccaagatataacaagcaaggagaggaaggataaaaatcaattttcaaaagattggaaccatattttcgaaggttccaaagaattccgtctccacccagagttcgcagttagctacaggacacctattgaagggtatccggtacagaagaggtacgggcgtctgcagttcgtttgcgcttttaatgcacacatcgttccacaaaatggtgagttcatcaatttgaaaaagcagatcgagaactttaacgatgaagacgttcagaaacgtaatgtgactgaattcaataaaaaggtgaatcatgcactttccgacaaagaatacgtcgttattggtattgatagaggcctcaaacagcttgccacactctgtgttttagacaaaagaggtaaaattcttggagattttgagatctacaaaaaggaatttgtgcgtgctgaaaaaagaagcgagagtcattgggaacacacacaagcagaaaccagacatatcttggatctttccaatttgcgtgtggagacaacaatagagggtaaaaaggttctcgtggaccagagcctcacacttgtgaaaaagaatcgtgatacaccagatgaggaagctactgaagaaaataaacagaaaatcaagttgaagcagctcagctatattagaaaattgcagcataagatgcagactaacgaacaggacgttttagatttaattaataatgaaccatcagatgaagaatttaagaaaagaatcgaggggcttatttccagttttggagaaggacagaagtacgctgaccttccaattaatactatgagagaaatgatctctgatctccagggagttatcgctagaggaaacaaccaaacagagaaaaataaaattattgaattagatgctgcagacaaccttaaacaaggtattgtagctaacatgatcggaattgttaattacatcttcgctaagtattcatacaaggcttacatctctcttgaggatttgtcaagagcctatggaggtgcaaagtccggttatgacggaaggtatctgccatcaacttcacaagacgaggatgtagatttcaaggaacagcagaatcagatgcttgcaggtttgggtacctaccaattcttcgagatgcagcttctgaaaaaacttcaaaagattcagagtgataacaccgttctgagattcgtgcccgctttcagatctgcagataactatagaaatattttgagacttgaggaaactaaatataagtctaagccgttcggcgttgttcatttcatagatccaaagtttacatcaaagaaatgccccgtctgtagcaaaacaaatgtatacagggacaaggatgacatcttggtttgcaaagagtgcggttttaggagcgactcccaattaaaagaaagagagaataacattcattatattcacaacggggacgataacggtgcataccacatcgcccttaagagcgttgagaatcttattcagatgaagtaa

SEQ ID NO: 157 - protein, 1232 aa, Sulfuricurvum sp. PC08-66; identical to SEQ ID NO: 155

SEQ ID NO: 158 - DNA, 4995 bp, Artificial Sequence
  pMDT452 MAD7d-AID-UGI nucleotide sequence
atgaataatggcacaaataacttccagaacttcattggcattagcagcctgcaaaaaacactgagaaatgcactgattccgacagaaacaacacagcagtttattgtcaaaaacggcatcatcaaagaggatgaactgagaggcgaaaatcgccaaattctgaaagatatcatggacgactattaccgtggctttatttcagaaacactgtccagcattgatgatatcgattggacaagcctgttcgagaaaatggaaatccaactgaaaaacggcgataacaaagacacgctgattaaagaacaaacggaatatcgcaaagcgatccacaaaaagtttgcaaatgatgaccgctttaaaaacatgttcagcgcgaaactgattagcgatattctgccggaatttgtcatccacaataataactatagcgcgagcgagaaagaagaaaaaacacaggtcattaaactgtttagccgctttgccacaagcttcaaagactatttcaaaaatcgcgcaaactgctttagcgcagatgatatttcatcatcaagctgccatcggattgtcaatgataatgcggaaatcttttttagcaacgcactggtctatcgcagaattgttaaatcattgagcaacgacgacatcaacaaaatctcaggcgatatgaaagacagcctgaaagaaatgtcactggaagaaatctacagctacgaaaaatacggcgaatttatcacacaagaaggcatcagcttttacaacgatatttgcggcaaagtcaacagctttatgaatctgtattgccagaaaaacaaagaaaacaaaaacctgtataaactgcagaaactgcacaagcagattctgtgcattgcagatacatcatatgaagtcccgtacaaatttgagagcgacgaagaagtttatcaaagcgttaatggctttctggataacatcagcagcaaacatattgttgaacgcctgagaaaaattggcgataactataatggctacaacctggacaaaatctacatcgtcagcaaattttacgaaagcgtcagccaaaaaacatatcgcgattgggaaacaattaatacagcgctggaaattcattataacaacattctgcctggcaacggcaaaagcaaagcagataaagttaaaaaggcggtcaaaaatgacctgcagaaaagcattacagaaatcaatgaactggtcagcaactacaaactgtgctcagatgataatatcaaggcggaaacgtacatccatgaaattagccatatcctgaacaactttgaagcgcaagaactgaaatataacccggaaatccatctggttgaaagcgaactgaaagcaagcgagctgaaaaatgttctggatgtcattatgaatgcgtttcattggtgcagcgtctttatgacagaagaactggtcgataaagataacaacttttatgcggaactggaagagatttacgacgaaatttatccggtcatcagcctgtataatctggttcgcaattatgtcacacagaaaccgtatagcacgaagaaaatcaaactgaactttggcattccgacactggcagatggctggtcaaaatcaaaagaatatagcaacaacgcgatcatcctgatgcgcgataatctttattatctgggcattttcaacgcgaaaaacaagccggacaaaaaaatcatcgaaggcaatacgtcagagaacaaaggcgactataaaaagatgatctataatctgcttccgggaccgaataaaatgatcccgaaagtttttctgtcaagcaaaacaggcgtcgaaacatataaaccgtcagcgtatattctggaaggctacaaacagaacaaacacatcaaaagcagcaaggactttgacatcacattttgccatgatctgatcgactactttaagaactgcattgcaattcatccggaatggaaaaacttcggctttgatttttcagacacgagcacgtatgaagatatcagcggcttttatagagaagttgaactgcagggctataaaatcgactggacatatatcagcgaaaaggatattgatctgctgcaagaaaaaggccaactgtacctgtttcagatctacaacaaagacttcagcaaaaaaagcacgggcaatgataacctgcatacgatgtacctgaaaaacctttttagcgaagagaacctgaaagacattgtcctgaaactgaatggcgaagccgaaattttctttcgcaaatccagcattaaaaacccgatcatccataaaaaaggcagcattctggttaaccgcacatatgaagcggaagaaaaagatcagtttggcaacattcagatcgtccgcaaaaacattccggaaaacatttatcaagaactgtacaaatactttaacgataaaagcgataaagaactgtccgacgaagcagcgaaacttaaaaatgttgttggccatcatgaagcggcaacaaacattgttaaagactatcgctatacgtacgataaatactttctgcatatgccgatcacgatcaacttcaaagcaaataaaacgggctttatcaacgatcgcattctgcagtatattgccaaagaaaaggatctgcatgtcatcggcattgctagaggcgaacgcaatctgatttatgtcagcgttattgatacatgcggcaacattgtcgaacagaaaagctttaacattgtcaacggctatgactaccagatcaagctgaaacagcaagaaggcgcaagacaaattgctcgcaaagaatggaaagaaatcggcaagatcaaagaaattaaagagggctatctgagcctggtcattcatgaaatttctaaaatggtcatcaaatataacgcgattatcgccatggaagatctgtcatatggctttaagaaaggccgttttaaagtcgaaagacaggtctaccagaaattcgaaacaatgctgattaacaaactgaattatctggtgtttaaagacatcagcatcacggaaaatggcggactgctgaaaggctatcaactgacatatattccggataagcttaaaaacgtcggccatcaatgcggctgcatcttttatgttccggcagcgtatacatcaaaaattgatccgacaacaggctttgtcaacatcttcaaattcaaagatctgacggtcgatgcgaaacgcgaattcattaagaaatttgacagcatccgctacgacagcgagaaaaatcttttctgctttacgttcgactacaacaactttatcacgcagaatacggttatgtcaaaaagcagctggtcagtctatacatatggcgttagaattaaacgcagatttgtgaacggcagatttagcaatgaaagcgatacaatcgacatcacgaaagacatggaaaaaacgcttgaaatgacggatattaactggcgtgatggacatgatcttcgccaggatattatcgattatgaaatcgtccagcacatctttgaaatctttagactgacagtccaaatgcgcaattcactgtcagaacttgaagatagagattatgatcgcctgatttctccggtcctgaatgaaaataacatcttttacgatagcgcaaaagcaggcgacgcactgccgaaagatgcggatgcaaatggcgcatattgcattgcactgaaaggcctgtatgaaatcaaacaaatcaccgagaattggaaagaggacggcaaattttcacgggataaactgaaaatcagcaacaaggactggtttgacttcatccaaaataagcgctacctgccgtcaagagcagatccgaagaaaaagagaaaagttggcggaggcggatcaggcggaggtggctcagcagaatatgttagagcactgtttgattttaacggcaacgatgaagaagatctgccgttcaaaaaaggcgatattctgagaattcgcgacaaaccggaagaacaatggtggaatgcagaagatagcgaaggcaaaagaggcatgattccggttccgtatgttgaaaaatactcaggcgattacaaagatcatgacggcgactataaagaccatgacatcgattataaggacgacgatgataaaagcagaatgacggatgcggaatatgttcgcattcatgaaaaactggacatctacacgttcaagaagcagttcttcaacaacaaaaaaagcgtcagccatagatgctacgttctgtttgaactgaaaagaagaggcgaaagacgcgcatgcttttggggctatgcagttaataaaccgcaatcaggcacagaacgcggaattcatgcagaaatctttagcattcgcaaagtcgaagaatatctgagagataatccgggacagtttacgattaattggtattcatcatggtcaccgtgcgcagattgcgcagaaaaaattctggaatggtataaccaagaactgagaggcaatggccatacactgaaaatttgggcatgcaaactgtactacgaaaaaaatgcacgcaatcaaattggcctgtggaatctgcgcgataatggcgttggcctgaatgttatggttagcgaacattatcaatgctgccgcaaaatctttattcagagcagccataatcagctgaatgaaaatagatggctggaaaaaacactgaaacgtgcggaaaaaagacgctcagaactgagcattatgatccaggttaaaatcctgcatacaacgaaatcaccggcagtttcaagaggctcaggcacaaatctgagcgatattatcgaaaaagaaacgggcaaacagctggtcattcaagaatcaattctgatgctgccggaagaagttgaagaagtcattggcaataaaccggaaagcgatatcctggttcatacagcatatgatgaaagcacagatgaaaatgtcatgctgctgacatcagatgcaccggaatacaaaccgtgggcacttgttattcaagatagcaatggcgagaacaagatcaaaatgctgtaa

SEQ ID NO: 159 - protein, 1664 aa, Artificial Sequence
  pMDT452 MAD7d-AID-UGI polypeptide sequence
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIARGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLPSRADPKKKRKVGGGGSGGGGSAEYVRALFDFNGNDEEDLPFKKGDILRIRDKPEEQWWNAEDSEGKRGMIPVPYVEKYSGDYKDHDGDYKDHDIDYKDDDDKSRMTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAVSRGSGTNLSDIIEKETGKQLVIQESILMLPEEVEEVIGNKPESDILVHTAYDESTDENVMLLTSDAPEYKPWALVIQDSNGENKIKML

SEQ ID NO: 160 - DNA, 7682 bp, Artificial Sequence
  pMDT454 nucleotide sequence
aactaactcaacgctagtagtggatttaatcccaaatgagccaacagaaccagaaccagaaacagaatcagaacaagtaacattggatttagaaatggaagaagaaaaaagcaatgacttcgtgtgaataatgcacgaaatcgttgcttattttttttaaaagcggtatactagatataacgaaacaacgaactgaatagaaacgaaaaaagagccatgacacatttataaaatgtttgacgacattttataaatgcatagcccgataagattgccaaaccaacgcttatcagttagtcagatgaactcttccctcgtaagaagttatttaattaactttgtttgaagacggtatataaccgtactatcattatatagggaaatcagagagttttcaagtatctaagctactgaatttaagaattgttaagcaatcaatcggaaatcgtttgattgctttttttgtattcatttatagaaggtggagtttgtatgaatcatgatgaatgtaaaacttatataaaaaatagtttattggagataagaaaattagcaaatatctatacactagaaacgtttaagaaagagttagaaaagagaaatatctacttagaaacaaaatcagataagtatttttcttcggagggggaagattatatatataagttaatagaaaataacaaaataatttattcgattagtggaaaaaaattgacttataaaggaaaaaaatctttttcaaaacatgcaatattgaaacagttgaatgaaaaagcaaaccaagttaattaaacaacctattttataggatttataggaaaggagaacagctgaatgaatatcccttttgttgtagaaactgtgcttcatgacggcttgttaaagtacaaatttaaaaatagtaaaattcgctcaatcactaccaagccaggtaaaagcaaaggggctatttttgcgtatcgctcaaaatcaagcatgattggcggtcgtggtgttgttctgacttccgaggaagcgattcaagaaaatcaagatacatttacacattggacacccaacgtttatcgttatggaacgtatgcagacgaaaaccgttcatacacgaaaggacattctgaaaacaatttaagacaaatcaataccttctttattgattttgatattcacacggcaaaagaaactatttcagcaagcgatattttaacaaccgctattgatttaggttttatgcctactatgattatcaaatctgataaaggttatcaagcatattttgttttagaaacgccagtctatgtgacttcaaaatcagaatttaaatctgtcaaagcagccaaaataatttcgcaaaatatccgagaatattttggaaagtctttgccagttgatctaacgtgtaatcattttggtattgctcgcataccaagaacggacaatgtagaattttttgatcctaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttaatagggcgtaataacgtcatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagtgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaagtaaaaaaagaagaaaaagaaagctatataaaggcgctgacaaattcttttgacttagagcatacattcattcaagagactttaaacaagctagcagaacgccctaaaacggacacacaactcgatttgtttagctatgatacaggctgaaaataaaacccgcactatgccattacatttatatctatgatacgtgtttgttttttctttgctgtttagcgaatgattagcagaaatatacagagtaagattttaattaattattagggggagaaggagagagtagcccgaaaacttttagttggcttggactgaacgaagtgagggaaaggctactaaaacgtcgaggggcagtgagagcgaagcgaacacttgattttttaattttctatcttttataggtcattagagtatacttatttgtcctataaactatttagcagcataatagatttattgaataggtcatttaagttgagcatattagaggaggaaaatcttggagaaatatttgaagaacccgattacatggattggattagttcttgtggttacgtggtttttaactaaaagtagtgaatttttgatttttggtgtgtgtgtcttgttgttagtatttgctagtcaaagtgattaaatagaattcatatccaatttatttttttcttaacaagggaggtgttttttaacatgactaaagtagggtatgcacgtgtcagtagcaaagaacagaacttagatagacaactgaaagcgttagagggcgtttctaaggtcttttcagacaaagcaagcggtcaatcggtcgaacgcccacaattacaagctatgcttaactatattcgtgaaggggatatagttgttgttactgaattagatcgattaggacgaaataataaagaattaacagaattgatgaatcaaattcaaattaagggggcaaccctggaagtcttaaatttaccctcaatgaatggtattgaagatgaaaatttaagacggctgattaataatttagtgattgaattgtataagtaccaagcggaatctgaacgcaaacgaattaaagaacgccaagcccaaggaattgaaattgctaagaaaaaaggaaaattcaaagggcgacaactgaaattcaaagaaaatgatccacgtttacaacacgctttcgatttgtttttgaacggtttatccgataaagaagttgaagaacaaactggaattaatcgccgaacgtttagaaggtatcgatcaagatacaacgtgacagtcgatcaaagaaaaaacaatgaaaagagggatagttaatgagtacggttattttagctgaaaaaccaagccaggcattagcctatgcaagtgctttaaaacaaagcaccaaaaaagacggttattttgagatcaaagacccaatctttgcagatgaaacgtttatcacgtttggttttgggcatttagtcgagttagcagaaccaggtcattatgacgaaaagtggcaaaattggaaacttgaatcattgccgatttttcctgatcgatacgattttgaagtggcaacagataaaaaaaagcagtttaaaattgttgctgaacttttaaaacaagcaaatacaatcattgtcgcaacagatagcgacagagaaggcgaaaacattgcctggtcgatcattcataaagcaaatgccttttctaaagataaaacgtataaaagactatggatcaatagtttagaaaaagatgtgatccgtagcggttttcaaaatttgcaaccaggaatgaattactatcccttttatcaagaagcgcaaacacgccaaattgccgattggttgatcggcatgaatgcaagccctttgtatacgttaaatttacagcagaagggcgtacaaggtacattttcactaggacgtgttcaaacgcccaccttatatcttatttttcagcgccaggaagccatagaaaactttagaaaagaaccttttttcgaggtggaagctagtataaaagtaaaccaagggtcatttaagggcgttataagccccacacagcgctttaaaacccaagaggagcttttagcttttgtttcttctgaacaagctaaaataggcaatcaagaggggataattgctgatgttcaaaccaaagagaagaaaacgaatagtccgagtttgttttctttaagtagtttgcaatcaaaagttaatcagctttataaagcgacagcgagccaaactttaaaagctatgcaaggactgtatgaagcaaaattattgagttatccaagaacagatacaccatttattacagagaacgaatttgcttatttaaaagcgaattttggcaaatatagcggttttttaggacttgatcttgaaatggttcaaacagagcctagaaagcgttatgtggacggtagtaaggtacaggaacaccacgccattatcccaacaaaacaagtacctaccgaatctgcattagcgaaaatggacgatttacaacgaaaaatatatgctttagtcgttaaaacgaccgttgccatgtttctacctgattatttgtatgaagaaactaagatacaaaccaaagtagccgacttactttttcaatcaataggcaagacaccaaagcaagaaggttggaaaattcttttcaaacaacaaaccaaagaagaagaagaggacgttcaaacgttacccttggttatcattggagaacatgccgaggttgacgttaagagtgccgaaaaagaaacacaaccaccgaaagcttttacagagggtacattattaactgctatgaaaacggcgaataaaacggttgatgatgaagaagcaatcaagattttacaagaagttgaggggattggaacagaagcgacaagagcaagcattattgaagccttgaaacaaaaagaatatatccaagtgattaagaataagcttgttgtaactgaaaaaggaaaattattgtgccaggcagttgaaagtcagcaccttttaacgagtgctgaaatgacggctaaatgggaaacgtatttaaaaaaaatcggtaaaagagaaggcaatcaagagaactttattacgaatatcaaaaaattcattgttcatttactggaagctgtacctaacgatatagaaaaactaaatttttctgattaccaggaacagaaagaaaaagaagcagaaaaaagtattgtaggaaaatgtcctaagtgtggcaacaatattgtattaaaaaaatcgttttatggttgttcaaattatcctgaatgtaagtttactttagctgaacattttagaaagaaaaaactcaccaaaacaaatgtaaaagaattactagagggaaaagaaaccctggtaaaaggaatcaaaacgaaagatagaaagtcctacaatgccgttgtaaaaatcggagaaaagggatatattgattttatatctttctcaaaataaacataaaagccctttaaagagggcttttatatattaatcacaaatcacttatcacaaatcacaagtgatttgtgattgttgatgataaaataagaataagaagaaatagaaagaagtgagtgattgtgggaaatttaggcgcacaaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagataaattattaggtatactactgacagcttccaaggagctaaagagctggcgaaagggggatgtgctgcaaggcgattaagttgggtaacgccagggttttcccagtcacgacgttgtaaaacgacggccagtgaattgatcaagctttaaatgcatgctagcaacgcggccgcgttgctagcatgcatttaaagcttgatcaattcgagctcattattaatctgttcagcaatcgggcgcgattgctgaataaaagatacgagagacctctcttgtatcttttttattttgagtggttttgtccgttacactagaaaaccgaaagacaataaaaattttattcttgctgagtctggctttcggtaagctagacaaaacggacaaaataaaaattggcaagggtttaaaggtggagattttttgagtgatcttctcaaaaaatactacctgtcccttgctgatttttaaacgagcacgagagcaaaacccccctttgctgaggtggcagagggcaggtttttttgtttcttttttctcgtaaaaaaaagaaaggtcttaaaggttttatggttttggtcggcactgccgacagcctcgcagagcacacactttatgaatataaagtatagtgtgttatactttacttggaagtggttgccggaaagagcgaaaatgcctcacattgtcgacggtatcgataagcttcccatactgaaactgcggactatctacaagagtagaaattaaaaaggtcttttgaccattttcttatacaaattatattatacatatcagtaaaataatgtcaacccccctttattccttttttttacacagcggacagtctggacagca

SEQ ID NO: 161 - DNA, 21 bp, Artificial Sequence
  PS1-AID protospacer pMDT454
agtccgcagtttcagtatggg

SEQ ID NO: 162 - DNA, 21 bp, Artificial Sequence
  PS2-AID protospacer pMDT455
agatgtcccaagcaaacggca

SEQ ID NO: 163 - DNA, 1743 bp, Artificial Sequence
  DsRed expression cassette
ataaatgagtagaaagcgccatatcggcgcttttcttttggaagaaaatatagggaaaatggtacttgttaaaaattcggaatatttatacaatatcatatgtatcacattgaaaggaggggcctgctgtccagactgtccgctgtgtaaaaaaaaggaataaaggggggttgacattattttactgatatgtataatataatttgtataagaaaatggaggggccctcgaaacgtaagatgaaaccttagataaaagtgctttttttgttgcaattgaagaattattaatgttaagcttaattaaagataatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatttatgaaaaatcattttatcagtttgaaaattatgtattatggagctcttataaaaatgaggagggaaccgaatggcttcaactgaagacgtaatcaaagagttcatgcgcttcaaagtgcgaatggaaggaagtgtaaacgggcatgagtttgaaattgaaggtgaaggtgaaggaaggccttatgaaggaacgcaaactgcaaaacttaaagtgacaaaaggaggaccgctgccgtttgcttgggacatcttaagtccgcagtttcagtatgggtcaaaagtttatgtaaagcatcctgctgacattcctgattacaaaaagttaagttttcctgaaggattcaagtgggagcgcgtaatgaactttgaagatggaggtgtcgtaactgtaacgcaagattcaagtctgcaagacggttgcttcatttacaaagtaaagttcattggcgtgaactttccaagtgatggtcctgtaatgcagaaaaagacaatgggttgggagccgtcaactgagaggctttatccgcgtgatggtgtcttgaaaggtgaaattcacaaagccttaaagttgaaagatggagggcattatcttgttgagttcaagagcatttacatggcgaaaaagcctgtgcagcttcctggctactactatgttgattcaaaacttgacataactagtcacaacgaagactacacaattgttgagcagtatgagcgaactgaaggaaggcatcatctttttctttaagagaccagacttccaattgacactaaagggatccagaagcggcaacacgctaatcaataaaaaaacgctgtgcggttaaagggcacagcgttttttgtgtatgaatcgaaaaagaggagagatcgcactgataattgccaacacaattaacatctcaatcaaggtaaatgctagcgcggccgcgtcgacaggcc

SEQ ID NO: 164 - DNA, 678 bp, Artificial Sequence
  DsRed coding region
atggcttcaactgaagacgtaatcaaagagttcatgcgcttcaaagtgcgaatggaaggaagtgtaaacgggcatgagtttgaaattgaaggtgaaggtgaaggaaggccttatgaaggaacgcaaactgcaaaacttaaagtgacaaaaggaggaccgctgccgtttgcttgggacatcttaagtccgcagtttcagtatgggtcaaaagtttatgtaaagcatcctgctgacattcctgattacaaaaagttaagttttcctgaaggattcaagtgggagcgcgtaatgaactttgaagatggaggtgtcgtaactgtaacgcaagattcaagtctgcaagacggttgcttcatttacaaagtaaagttcattggcgtgaactttccaagtgatggtcctgtaatgcagaaaaagacaatgggttgggagccgtcaactgagaggctttatccgcgtgatggtgtcttgaaaggtgaaattcacaaagccttaaagttgaaagatggagggcattatcttgttgagttcaagagcatttacatggcgaaaaagcctgtgcagcttcctggctactactatgttgattcaaaacttgacataactagtcacaacgaagactacacaattgttgagcagtatgagcgaactgaaggaaggcatcatctttttctttaa

SEQ ID NO: 165 - DNA, 16 bp, Artificial Sequence
  Primer ID 1202334
ttgcaccgtctaatgg

SEQ ID NO: 166 - DNA, 20 bp, Artificial Sequence
  Primer ID 1228373
gatgatgccttccttcagtt

(19) United States
(12) Patent Application Publication
(10) Pub. No.: US 2025/0179528 A1
(43) Pub. Date: Jun. 5, 2025

(54) CRISPR-AID USING CATALYTICALLY INACTIVE RNA-GUIDED ENDONUCLEASE

(71) Applicant: Novozymes A/S, Bagsvaerd (DK)

(72) Inventors: Takashi Nakanishi, Chiba-shi (JP); Michael David Thomas, Davis, CA (US)

(73) Assignee: Novozymes A/S, Bagsvaerd (DK)

(21) Appl. No.: 17/910,166

(22) PCT Filed: Mar. 10, 2021
(86) PCT No.: PCT/US2021/021670

§ 371 Date: Sep. 8, 2022

(30) Foreign Application Priority Data
Mar. 12, 2020 (EP) 20162685.0

(51) Int. Cl. C12N 15/90; C12N 9/22; C12N 9/78; C12N 15/11; C12N 15/75; C12N 15/81

(52) U.S. Cl. CPC C12N 15/90; C12N 9/22; C12N 9/78; C12N 15/11; C12N 15/75; C12N 15/815; C12Y 305/04005; C12N 2310/20; C12N 2800/101; C12N 2800/102

(57) ABSTRACT

The present invention relates to nucleobase editing complexes comprising a) a catalytically inactive RNA-guided endonuclease having a sequence identity of at least 60% to SEQ ID NO: 126 or SEQ ID NO: 155 and b) a nucleobase editing domain, as well as polynucleotides encoding said nucleobase editing complexes, nucleic acid constructs and expression vectors comprising said polynucleotides, host cells comprising said nucleobase editing complexes and/or polynucleotides, and methods for preparing and using said nucleobase editing complexes.

### REFERENCE TO A SEQUENCE LISTING

This application contains a Sequence Listing in computer readable form, which is incorporated herein by reference.

### FIELD OF THE INVENTION

The present invention relates to nucleobase editing complexes comprising a) a catalytically inactive RNA-guided endonuclease having a sequence identity of at least 60% to SEQ ID NO: 126 or SEQ ID NO: 155 and b) a nucleobase editing domain, as well as polynucleotides encoding said nucleobase editing complexes, nucleic acid constructs and expression vectors comprising said polynucleotides, host cells comprising said nucleobase editing complexes and/or polynucleotides, and methods for preparing and using said nucleobase editing complexes.

### BACKGROUND OF THE INVENTION

Introduction of single nucleotide polymorphisms (SNPs, also known as single nucleotide variations, SNVs) into genomes is key for evolution of a given organism. Industrially important microbes are often mutagenized in a laboratory to obtain new or enhanced functions such as stress tolerance, higher expression potential of a molecule of interest, and so forth. However, the existing methods for SNP introduction have notable limitations. Random mutagenesis obtained via UV irradiation or chemical exposure provides highly diversified genotypes, but identification of beneficial SNPs is labor intensive. Site-directed mutagenesis obtained via the CRISPR technology allows introduction of target-specific SNPs and thus seems more promising. Briefly, an RNA-guided endonuclease such as Cas9 or Cpf1 is directed to a locus of interest using a guide-RNA (gRNA) that has a protospacer region complementary to a DNA sequence in the locus of interest. The RNA-guided endonuclease binds and cuts the DNA strand at the target locus, leading to a double-stranded break (DSB) that the cell will attempt to repair using non-homologous end joining (NHEJ) or similar mechanisms. However, if the cell is supplied with repair DNA that encode a desired point mutation and is flanked by sequences homologous to the regions upstream and downstream of the DSB, homology-directed repair (HDR) will result in incorporation of the repair DNA containing the desired mutation in the locus of interest. Although widely applied, this CRISPR-based approach is not suited for large-scale mutagenesis studies since repair DNA would be needed for every single target locus. Moreover, the introductions of DSBs is associated with cytotoxicity. Hence, a method of generating highly diverse mutants in a targetable manner and with easy identification of the resulting SNPs would be very advantageous.

The requirement for repair DNA may be circumvented by using the so-called CRISPR-AID technology (Komor et al., Nature, vol. 533, pp. 420-424, 2016; Nishida et al., Science, vol. 353, aaf8729, 2016). Herein, a catalytically inactive RNA-guided endonuclease is tethered to a nucleobase editing domain to form a nucleobase editing complex. The nucleobase editing domain is either a cytosine base editor (CBE) that converts C-G base pairs to T-A base pairs or an adenine base editor (ABE) that converts A-T base pairs into G-C base pairs (Rees and Liu, Nat. Rev. Genetics, vol. 19, pp. 770-788, 2018). Binding of the catalytically inactive RNA-guided endonuclease to the target locus via base pairing between the gRNA protospacer and its complementary DNA sequence leads to displacement of a small segment of single-stranded DNA in a so-called “R loop”, wherein the nucleobases are exposed to deamination by the nucleobase editing domain, resulting in transition mutations and SNP generation. Importantly, since the nucleobase editing complex is adapted to operate only on single-stranded DNA, DSBs do not occur and the associated cytotoxic side-effects are avoided. CRISPR-AID is thus a useful methodology for introducing SNPs into a genome of interest in a scalable and targetable manner.

Catalytically inactive variants of Cas9 and Cpf1 have been widely applied in the CRISPR-AID technology (Nishida et al., vide supra; Li et al., Nat. Biotech., vol. 36, pp. 324-327, 2018). However, further developments of CRISPR-AID systems are still warranted to alter or improve various properties such as editing efficiency or editing window.

WO 2015/133554 describes CRISPR-AID utilizing Cas9d as the catalytically inactive RNA-guided endonuclease and activation-induced cytidine deaminase (AID) as nucleobase editing domain.

### SUMMARY OF THE INVENTION

The present inventors have investigated the use of catalytically inactive versions of the RNA-guided endonuclease known as Mad7 isolated from Eubacterium rectale in CRISPR-AID base editing. Mad7 has only 31% sequence identity to Cpf1 isolated from Acidominococcus sp. and is thus structurally very different from other known RNA-guided endonucleases. However, as illustrated in the Examples disclosed herein, CRISPR-AID base editing utilizing Mad7d-AID provided editing efficiency comparable to Cas9d-AID in microbial host cells transformed with a single plasmid containing Mad7d-AID and gRNA expression cassettes for stable expression of Mad7d-AID components. Moreover, the editing window observed for Mad7d-AID was broader than for Cas9d-AID, illustrating that Mad7d-based CRISPR-AID is more suitable for generating multiple different SNPs of the same target locus, which is highly beneficial for generating SNP libraries and for screening purposes.

In a first aspect, the present invention relates to nucleobase editing complexes comprising, consisting essentially of, or consisting of:
a) a catalytically inactive RNA-guided endonuclease having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126; and
b) a nucleobase editing domain.

In a second aspect, the present invention relates to polynucleotides encoding nucleobase editing complexes of the first aspect.

In a third aspect, the present invention relates to nucleic acid constructs comprising polynucleotides of the second aspect.

In a fourth aspect, the present invention relates to expression vectors comprising polynucleotides of the second aspect and/or nucleic acid constructs of the third aspect.

In a fifth aspect, the present invention relates to host cells comprising nucleobase editing complexes of the first aspect, polynucleotides of the second aspect, nucleic acid constructs of the third aspect, and/or expression vectors of the fourth aspect.

In a sixth aspect, the present invention relates to methods for modifying at least one nucleobase in a DNA target sequence, the method comprising:
a) providing a nucleobase editing complex of the first aspect complexed with a gRNA that is complementary to and capable of hybridizing to the DNA target sequence; and
b) contacting the nucleobase editing complex with the DNA target sequence;

wherein at least one nucleobase in the DNA target sequence is converted to a different nucleobase without introducing a double-strand break in the DNA sequence of interest.

### BRIEF DESCRIPTION OF FIGURES

FIG. 1 shows a schematic illustration of CRISPR-AID mode of action.

FIG. 2 shows a schematic drawing of the plasmid pHite277.

FIG. 3 shows a schematic drawing of the plasmid pTNA193.

FIG. 4 shows genome sequences of white spore mutants generated by dCas9-AID.

FIG. 5 shows a schematic drawing of the plasmid pTNA235.

FIG. 6 shows genome sequences of white spore mutants generated by dCas9-AID-UGI.

FIG. 7 shows a schematic drawing of the plasmid pTNA287.

FIG. 8 shows genome sequences of white spore mutants generated by Mad7d-AID-UGI.

FIG. 9 shows genome sequences of white spore mutants generated by 34° C. incubation.

FIG. 10 shows a schematic drawing of the plasmid pAT3530.

FIG. 11 shows a schematic drawing of the plasmid pMDT452.

FIG. 12 shows a schematic drawing of the plasmid pMDT454/pMDT455.

FIG. 13 shows a schematic drawing of the DsRed expression cassette of strain MDT545.

### DEFINITIONS

In accordance with this detailed description, the following definitions apply. Note that the singular forms “a,” “an,” and “the” include plural references unless the context clearly dictates otherwise.

Reference to “about” a value or parameter herein includes aspects that are directed to that value or parameter per se. For example, description referring to “about X” includes the aspect “X”.

Unless defined otherwise or clearly indicated by context, all technical and scientific terms used herein have the same meaning as commonly understood by one of ordinary skill in the art to which this invention belongs.

Catalytically inactive: The term “catalytically inactive” is used to described RNA-guided endonucleases for which endonuclease activity has been disrupted. A catalytically inactive endonuclease can bind to but not introduce any breaks in a target DNA sequence. The terms “catalytically inactive”, “nuclease-null” and “dead” (abbreviated “d”, e.g., Mad7d) are used interchangeably herein.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a polypeptide of the present invention. Each control sequence may be native (i.e., from the same gene) or heterologous (i.e., from a different gene) to the polynucleotide encoding the polypeptide or native or heterologous to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” means any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fusion polypeptide: The term “fusion polypeptide” is a polypeptide in which one polypeptide is fused at the N-terminus or the C-terminus of a polypeptide of the present invention. A fusion polypeptide is produced by fusing a polynucleotide encoding another polypeptide to a polynucleotide of the present invention. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12:2575-2583; Dawson et al., 1994, Science 266:776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3:568-576; Svetina et al., 2000, J. Biotechnol. 76:245-251; Rasmussen-Wilson et al., 1997, Appl. Environ. Microbiol. 63:3488-3493; Ward et al., 1995, Biotechnology 13:498-503; and Contreras et al., 1991, Biotechnology 9:378-381; Eaton et al., 1986, Biochemistry 25:505-512; Collins-Racie et al., 1995, Biotechnology 13:982-987; Carter et al., 1989, Proteins: Structure, Function, and Genetics 6:240-248; and Stevens, 2003, Drug Discovery World 4:35-48.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, or domain of a polypeptide or nucleic acid is not naturally associated with the polypeptide or nucleic acid, i.e., the control sequence is from a gene other than the gene encoding the mature polypeptide of SEQ ID NO: 126.

Host cell: The term “host cell” means any microbial or plant cell into which a nucleic acid construct or expression vector comprising a polynucleotide of the present invention has been introduced. Methods for introduction include but are not limited to protoplast fusion, transfection, transformation, electroporation, conjugation, and transduction. In some embodiments, the host cell is an isolated recombinant host cell that is partially or completely separated from at least one other component with, including but not limited to, proteins, nucleic acids, cells, etc.

Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture broth containing the secreted polypeptide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

RNA-guided endonuclease: The term “RNA-guided endonuclease” means a polypeptide having endonuclease activity, wherein the endonuclease activity is controlled by one or more gRNA that form a complex with the RNA-guided endonuclease and directs the endonuclease activity to a target DNA sequence that is complementary to and capable of hybridizing to the protospacer region(s) of the one or more gRNA.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the—nobrief option must be specified in the command line. The output of Needle labeled “longest identity” is calculated as follows: (Identical Residues×100)/(Length of Alignment-Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

$$( Identical Deoxyribonucleotides \times 100 ) / ( Length of Alignment - Total Number of Gaps in Alignment )$$

### Conventions for Designations of Variants

For purposes of the present invention, the polypeptide disclosed in SEQ ID NO: 126 is used to determine the corresponding amino acid position in another RNA-guided endonuclease. The amino acid sequence of another RNA-guided endonuclease is aligned with the polypeptide disclosed in SEQ ID NO: 126, and based on the alignment, the amino acid position number corresponding to any amino acid residue in the polypeptide disclosed in SEQ ID NO: 126 is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix.

In describing the variants of the present invention, the nomenclature described below is adapted for ease of reference. The accepted IUPAC single letter or three letter amino acid abbreviation is employed.

Substitutions: For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine(S) with phenylalanine (F), respectively.

Deletions: For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions: For an amino acid insertion, the following nomenclature is used: Original amino acid, position, original amino acid, inserted amino acid. Accordingly, the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations: Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively. Different alterations. Where different alterations can be introduced at a position, the different alterations are separated by a comma, e.g., “Arg170Tyr, Glu” represents a substitution of arginine at position 170 with tyrosine or glutamic acid. Thus, “Tyr167Gly, Ala+Arg170Gly,Ala” designates the following variants:
“Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The present inventors have investigated the use of catalytically inactive versions of the RNA-guided endonuclease known as Mad7 isolated from Eubacterium rectale in CRISPR-AID base editing. Mad7 has only 31% sequence identity to Cpf1 isolated from Acidominococcus sp. and is thus structurally very different from other known RNA-guided endonucleases. However, as illustrated in the Examples disclosed herein, CRISPR-AID base editing utilizing Mad7d-AID provided editing efficiency comparable to Cas9d-AID in microbial host cells transformed with a single plasmid containing Mad7d-AID and gRNA expression cassettes for stable expression of Mad7d-AID components. Moreover, the editing window observed for Mad7d-AID was broader than for Cas9d-AID, illustrating that Mad7d-based CRISPR-AID is more suitable for generating multiple different SNPs of the same target locus, which is highly beneficial for generating SNP libraries and for screening purposes.

### Nucleobase Editing Complexes

In a first aspect, the present invention relates to a nucleobase editing complex comprising, consisting essentially of, or consisting of:
a) a catalytically inactive RNA-guided endonuclease having a sequence identity of at least 60%, such as e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126 or SEQ ID NO: 155; and
b) a nucleobase editing domain.

In an embodiment, the nucleobase editing complex comprises, consists essentially of, or consists of:
a) a catalytically inactive RNA-guided endonuclease having a sequence identity of at least 60%, such as e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126; and
b) a nucleobase editing domain.

The catalytically inactive RNA-guided endonuclease may be any catalytically inactive RNA-guided endonuclease having at least 60% sequence identity to SEQ ID NO: 126 or SEQ ID NO: 155. In a preferred aspect, the catalytically inactive RNA-guided endonuclease comprises an alteration of an amino acid at a position corresponding to position 877 of SEQ ID NO: 126. In an embodiment, the amino acid at a position corresponding to position 877 of SEQ ID NO: 126 is substituted with Ala, Arg, Asn, Asp, Cys, Gln, Glu, Gly, His, 11e , Leu, Lys, Met, Phe, Pro, Ser, Thr, Trp, Tyr, or Val, preferably with Ala. In another embodiment, the variant comprises or consists of the substitution D877A of SEQ ID NO: 126. In a preferred embodiment, the catalytically inactive RNA-guided endonuclease comprises, consists essentially of, or consists of SEQ ID NO: 126. In another preferred embodiment, the catalytically inactive RNA-guided endonuclease comprises, consists essentially of, or consists of SEQ ID NO: 155. In one embodiment the catalytically inactive RNA-guided endonuclease is encoded by a polynucleotide having a sequence identity of at least 80%, such as e.g., at least 85%, at least 90%, at least 91%, at least 92%, at least 93%, at least 94%, at least 95%, at least 96%, at least 97%, at least 98%, at least 99%, or 100%, to SEQ ID NO: 156. Preferably the polynucleotide encoding the catalytically inactive RNA-guided endonuclease comprises or consists of the polynucleotide of SEQ ID NO: 156.

The nucleobase editing domain may be either a cytosine base editor (CBE) that converts C-G base pairs to T-A base pairs or an adenine base editor (ABE) that converts A-T base pairs into G-C base pairs. Collectively, CBEs and ABEs can mediate all four possible transition mutations (C to T, A to G, T to C, and G to A) of DNA nucleobases (Rees and Liu, vide supra). In an aspect, the nucleobase editing domain is a cytosine base editor (CBE). CBEs convert C-G base pairs to T-A base pairs by deaminating the exocyclic amine a target cytosine

(C) to generate uracil (U), which is read as thymine (T) by polymerase enzymes. Suitable CBEs include members of the APOBEC/AID family, in particular cytidine deaminase 1 (CDA1, such as PmCDA1 obtained from Petromyzon marinus encoded by the polynucleotide with SEQ ID NO: 127; Nishida et al., vide supra) and APOBEC1 (Harris et al., Mol. Cell., vol. 10, pp. 1247-1253, 2002), but also APOBEC2, APOBEC3, APOBEC4, and APOBEC5 (Knisbacher et al., Trends Genet., vol. 32, pp. 553-563, 2000).

In a preferred embodiment, the nucleobase editing domain is a cytosine base editor of the APOBEC/AID family.

In a preferred embodiment, the nucleobase editing domain is APOBEC1 or a homolog or variant thereof.

In a preferred embodiment, the nucleobase editing domain is CDA1 or a homolog or variant thereof; most preferably the nucleobase editing domain is PmCDA1 obtained from Petromyzon marinus.

In a preferred embodiment, the nucleobase editing domain comprises or consists of a polypeptide having a sequence identity of at least 80%, such as, e.g., at least 85%, at least 90%, at least 91%, at least 92%, at least 93%, at least 94%, at least 95%, at least 96%, at least 97%, at least 98%, at least 99%, or 100%, to SEQ ID NO: 128; preferably the nuclease editing domain comprises, consists essentially of, or consists of SEQ ID NO: 128.

In an aspect, the nucleobase editing domain is an adenine base editor (ABE). Similar to CBEs, ABEs convert A-T base pairs into G-C base pairs by deaminating the exocyclic amine of a target adenine (A) to generate inosine (I). In the context of a polymerase active site, I has a preference for base pairing with cytosine (C), which is then read or replicated as guanine (G). Suitable ABEs are based on tRNA adenosine deaminase (TadA) from E. coli (Gaudelli et al., Nature, vol. 551, p. 464-471, 2017) and include TadA, TadA* (a A106V and/or D108N variant of TadA), TadA homodimers, and TadA-TadA* heterodimers.

In a preferred embodiment, the nucleobase editing domain is an adenine base editor based on tRNA adenosine deaminase (TadA).

In a preferred embodiment, the nucleobase editing domain is selected from the group consisting of TadA, TadA*, TadA homodimer, and TadA-TadA* heterodimer; most preferably the nucleobase editing domain is a TadA-TadA* heterodimer.

A challenge for the use of nucleobase editing domains in eukaryotic cells, in particular mammalian cells, is the need to circumvent the DNA repair processes that are initiated upon formation of the A-U and I-T intermediates, in particular the base excision repair process that is activated by the U-G mismatch generated from the deamination of C to U by CBEs. The repair of U-G mismatches is initiated by uracil N-glycosylase (UNG), and this enzyme may be inhibited by uracil DNA glycosylase inhibitor (UGI), a DNA mimetic derived from the PBS bacteriophage (Mol et al., Cell, vol. 82, pp. 701-708, 1995).

In an embodiment, the nucleobase editing domain is a cytosine base editor (CBE), and the nucleobase editing complex further comprises an uracil DNA glycosylase inhibitor (UGI). Preferably, the uracil DNA glycosylase inhibitor has a sequence identity of at least 80%, such as e.g., at least 85%, at least 90%, at least 91%, at least 92%, at least 93%, at least 94%, at least 95%, at least 96%, at least 97%, at least 98%, at least 99%, or 100%, to SEQ ID NO: 132. Most preferably, the uracil DNA glycosylase inhibitor comprises, consists essentially of, or consists of SEQ ID NO: 132.

The catalytically inactive RNA-guided endonuclease and the nucleobase editing domain may be linked in various ways to form a nucleobase editing complex of the invention. In one embodiment, the RNA-guided endonuclease and the nucleobase editing domain are fused end-to-end, i.e., without an intervening polypeptide sequence. In another embodiment, the catalytically inactive RNA-guided endonuclease and the nucleobase editing domain are connected via an intervening polypeptide (i.e., a linker polypeptide). By arranging the catalytically inactive RNA-guided endonuclease and nucleobase editing domain end-to-end or connecting these via a linker polypeptide, one can express the nucleobase editing complex as a single fusion polypeptide. The length and amino acid composition of the linker polypeptide will depend on the size and the three-dimensional structure of the catalytically inactive RNA-guided endonuclease and the nucleobase editing domain, and the linker polypeptide should in general be of sufficient length and flexibility to prevent steric occlusion of the binding and/or active site(s) of the catalytically inactive RNA-guided endonuclease and the nucleobase editing domain when these are linked together.

Thus, in an aspect, the nucleobase editing complex is a fusion polypeptide comprising a catalytically inactive RNA-guided endonuclease and a nucleobase editing domain. Techniques for producing fusion polypeptide are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12:2575-2583; Dawson et al., 1994, Science 266:776-779).

In an embodiment, the catalytically inactive RNA-guided endonuclease and the nucleobase editing domain are fused end-to-end, i.e., with no intervening linker polypeptide.

In another embodiment, the catalytically inactive RNA-guided endonuclease and the nucleobase editing domain are separated by a linker polypeptide. Preferably, the catalytically inactive RNA-guided endonuclease, the linker polypeptide, and the nucleobase editing domain are encoded in frame and are expressed as a single polypeptide.

Preferably, the linker polypeptide comprises at least 10 amino acid residues, such as, e.g., at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 225, at least 250, at least 275, at least 300, at least 350, at least 400, at least 500, at least 600, at least 700, at least 800, at least 900, at least 1000, or more, amino acid resides. More preferably, the linker polypeptide comprises at least 75 amino acid residues, such as, e.g., at least 80, at least 85, at least 90, at least 95, at least 100, at least 105, at least 110, at least 115, at least 120, or at least 125 amino acid residues. Even more preferably, the linker polypeptide comprises 80-120 amino acid residues, such as, e.g., 85-115 amino acid residues, 90-110 amino acid residues, or 95-105 amino acid residues.

In a preferred embodiment, the linker comprises or consists of 16 amino acid residues.

In a preferred embodiment, the linker comprises or consists of 32 amino acid residues.

In a preferred embodiment, the linker comprises or consists of 100 amino acid residues.

In a preferred embodiment, the linker comprises or consists of 105 amino acid residues.

In an embodiment, the linker polypeptide has a sequence identity of at least 80%, such as e.g., at least 85%, at least 90%, at least 91%, at least 92%, at least 93%, at least 94%, at least 95%, at least 96%, at least 97%, at least 98%, at least 99%, or 100%, to SEQ ID NO: 130. Preferably, the linker polypeptide comprises or consists of the polypeptide of SEQ ID NO: 130. In one embodiment the linker polypeptide is encoded by a polynucleotide having a sequence identity of at least 80%, such as e.g., at least 85%, at least 90%, at least 91%, at least 92%, at least 93%, at least 94%, at least 95%, at least 96%, at least 97%, at least 98%, at least 99%, or 100%, to SEQ ID NO: 129. Preferably the polynucleotide encoding the linker polypeptide comprises or consists of the polynucleotide of SEQ ID NO: 129.

Another option for constructing a nucleobase editing complex of the invention is to express the catalytically inactive RNA-guided endonuclease and the nucleobase editing domain as two separate polypeptides and then subsequently connect these via chemical conjugation methods utilizing biocompatible, and preferably biorthogonal, small molecule reactions. Typically, the polypeptides are modified post-translationally by installing the required small molecule functionalities, which optionally may be attached to the polypeptide via a linker. A suitable conjugation method is the Cu(I)-catalyzed azide-alkyne cycloaddition (CuAAC; Rostovtsev et al., Angew. Chem. Int. Ed, vol. 41, pp. 2596-2599, 2002; Tornøe et al., J. Org. Chem, vol. 67, pp. 3057-3064, 2002).

In an embodiment, the nucleobase editing complex of the invention comprises:
a) a catalytically inactive RNA-guided endonuclease having a sequence identity of at least 60%, such as e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126; and
b) a nucleobase editing domain comprising a polypeptide having a sequence identity of at least 80%, such as, e.g., at least 85%, at least 90%, at least 91%, at least 92%, at least 93%, at least 94%, at least 95%, at least 96%, at least 97%, at least 98%, at least 99%, or 100%, to SEQ ID NO: 128.

In an embodiment, the nucleobase editing complex of the invention comprises:
a) a catalytically inactive RNA-guided endonuclease having a sequence identity of at least 60%, such as e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126;
b) a nucleobase editing domain comprising a polypeptide having a sequence identity of at least 80%, such as, e.g., at least 85%, at least 90%, at least 91%, at least 92%, at least 93%, at least 94%, at least 95%, at least 96%, at least 97%, at least 98%, at least 99%, or 100%, to SEQ ID NO: 128; and
c) an uracil DNA glycosylase inhibitor comprising a polypeptide having a sequence identity of at least 80%, such as e.g., at least 85%, at least 90%, at least 91%, at least 92%, at least 93%, at least 94%, at least 95%, at least 96%, at least 97%, at least 98%, at least 99%, or 100%, to SEQ ID NO: 132.

In an embodiment, the nucleobase editing complex of the invention comprises:
a) a catalytically inactive RNA-guided endonuclease having a sequence identity of at least 60%, such as e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126;
b) a nucleobase editing domain comprising a polypeptide having a sequence identity of at least 80%, such as, e.g., at least 85%, at least 90%, at least 91%, at least 92%, at least 93%, at least 94%, at least 95%, at least 96%, at least 97%, at least 98%, at least 99%, or 100%, to SEQ ID NO: 128; and
c) an uracil DNA glycosylase inhibitor comprising a polypeptide having a sequence identity of at least 80%, such as e.g., at least 85%, at least 90%, at least 91%, at least 92%, at least 93%, at least 94%, at least 95%, at least 96%, at least 97%, at least 98%, at least 99%, or 100%, to SEQ ID NO: 132, wherein the catalytically inactive RNA-guided endonuclease and the nucleobase editing domain is separated by a linker polypeptide comprising at least 50 amino acids; preferably at least 100 amino acids.

### Guide-RNA

The gRNA constitutes the re-programmable part of the CRISPR system that allows targeting of the RNA-guided endonuclease to a specific locus of interest. In most natural systems, including S. pyogenes, the gRNA is a complex of two RNA polynucleotides, a first RNA (crRNA or protospacer) containing about 20 nucleotides that determine the specificity of the RNA-guided endonuclease and a second RNA (tracrRNA or scaffold) which hybridizes to the first RNA to form an RNA complex that interacts with RNA-guided endonuclease (see Jinek et al., 2012, A programmable dual-RNA-guided DNA endonuclease in adaptive bacterial immunity, Science 337: 816-821). The terms crRNA and tracrRNA are used interchangeably with the terms tracr-mate RNA and tracr RNA herein.

Since the discovery of the CRISPR-Cas9 system, single polynucleotide gRNAs have been developed and successfully applied just as effectively as natural two-part gRNA complex.

In a preferred embodiment, the gRNA is a single gRNA or RNA complex comprising a first RNA comprising 20 or more nucleotides that are at least 85% complementary to and capable of hybridizing to the one or more DNA target sequence; preferably the 20 or more nucleotides are at least 90%, 95%, 97%, 98%, 99% or even 100% complementary to and capable of hybridizing to the one or more DNA target sequence.

In a particularly preferred embodiment, the gRNA is a single gRNA or RNA complex comprising a first RNA comprising 21 nucleotides that are at least 85% complementary to and capable of hybridizing to the one or more DNA target sequence; preferably the 21 are at least 90%, 95%, 97%, 98%, 99% or even 100% complementary to and capable of hybridizing to the one or more DNA target sequence.

In another preferred embodiment, the gRNA comprises the first and second RNAs in the form of a single polynucleotide, wherein the tracr mate sequence and the tracr sequence form a stem-loop structure when hybridized with each other.

### DNA Target Sequence

The DNA target sequence may be found anywhere, including in vivo (e.g., inside a cell or a living organism, including as part of the genome of said cell or organism), ex vivo, or in vitro. The DNA target sequence should be complementary to and capable of hybridizing to a gRNA suitable for directing the binding of the catalytically inactive RNA-guided endonucleases of the invention to the DNA target sequence.

Preferably, the DNA target sequence is at least 20 nucleotides in length in order to allow its hybridization to the corresponding at least 20 nucleotide sequence of the gRNA. The DNA target sequence can be located anywhere in the genome but will often be within a coding sequence or open reading frame.

In a preferred embodiment, the DNA target sequence comprises a polynucleotide comprising 20 or more nucleotides that are at least 85% complementary to and capable of hybridizing to the gRNA; preferably the 20 or more nucleotides are at least 90%, 95%, 97%, 98%, 99% or even 100% complementary to and capable of hybridizing to the gRNA.

In a particularly preferred embodiment, the DNA target sequence comprises a polynucleotide comprising 21 nucleotides that are at least 85% complementary to and capable of hybridizing to the gRNA; preferably the 21 nucleotides are at least 90%, 95%, 97%, 98%, 99% or even 100% complementary to and capable of hybridizing to the gRNA.

The DNA target sequence should be flanked by a functional protospacer adjacent motif (PAM) that is recognized by the RNA-guided endonuclease of the invention. For an overview of PAM sequences, see, for example, Shah et al., 2013, Protospacer recognition motifs, RNA Biol. 10 (5): 891-899. Preferably, the PAM sequence is 5′-TTTN-3′ or 5′-CTTN-3′. More preferably, the PAM sequence is 5′-TTTN-3′. Most preferably, the PAM sequence is 5′-TTTC-3′ or 5′-TTTG-3′.

In a preferred embodiment, the DNA target sequence is located at the 3′ end of the PAM sequence. Most preferably, the DNA target sequence is located directly adjacent to the 3′ end of the PAM sequence.

Preferably, the DNA target sequence is comprised in an open reading frame encoding a polypeptide or in a promoter region. Also preferably, the DNA target sequence encode one or more enzyme selected from the group consisting of hydrolase, isomerase, ligase, lyase, oxidoreductase, or a transferase; preferably the one or more enzyme is an alpha-amylase, alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucano-transferase, glucoamylase, invertase, laccase, lipase, mannosidase, mutanase, oxidase, pectinolytic enzyme, peroxidase, phosphodiesterase, phytase, polyphenoloxidase, proteolytic enzyme, ribonuclease, transglutaminase, and xylanase.

Preferably, the DNA target sequence encodes a fluorescent protein (e.g., green fluorescent protein), a fragment or a variant thereof.

### Polynucleotides

The present invention also relates to isolated polynucleotides encoding a nucleobase editing complex of the present invention, as described herein.

The techniques used to isolate or clone a polynucleotide are known in the art and include isolation from genomic DNA or cDNA, or a combination thereof. The cloning of the polynucleotides from genomic DNA can be affected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used.

### Nucleic Acid Constructs

The present invention also relates to nucleic acid constructs comprising a polynucleotide encoding a nucleobase editing complex of the present invention, wherein the polynucleotide is operably linked to one or more control sequences that direct the expression of the coding sequence in a suitable host cell under conditions compatible with the control sequences.

The polynucleotide may be manipulated in a variety of ways to provide for expression of the nucleobase editing complex. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis crylllA gene (Agaisse and Lereclus, 1994, Molecular Microbiology 13:97-107), E. coli lac operon, E. coli trc promoter (Egon et al., 1988, Gene 69:301-315), Streptomyces coelicolor agarase gene (dagA), and prokaryotic beta-lactamase gene (Villa-Kamaroff et al., 1978, Proc. Natl. Acad. Sci. USA 75:3727-3731), as well as the tac promoter (DeBoer et al., 1983, Proc. Natl. Acad. Sci. USA 80:21-25). Further promoters are described in “Useful proteins from recombinant bacteria” in Gilbert et al., 1980, Scientific American 242:74-94; and in Sambrook et al., 1989, supra. Examples of tandem promoters are disclosed in WO 99/43835.

Examples of suitable promoters for directing transcription of the polynucleotide of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8:423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rmnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C(CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis crylllA gene (WO 94/25612) and a Bacillus subtilis SP82 gene (Hue et al., 1995, J. Bacteriol. 177:3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15:5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiol. Rev. 57:109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide encoding a nucleobase editing complex of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98:61-67; Cullen et al., 1987, Nucleic Acids Res. 15:9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells comprising a polynucleotide encoding a nucleobase editing complex of the present invention operably linked to one or more control sequences that direct the expression of the nucleobase editing complex. A construct or vector comprising a polynucleotide is introduced into a host cell so that the construct or vector is maintained as a chromosomal integrant or as a self-replicating extra-chromosomal vector as described earlier.

In some embodiments, at least one of the one or more control sequences is heterologous to the polynucleotide encoding the nucleobase editing complex. In some embodiments, at least one of the one or more control sequences is heterologous to the host cell.

In some embodiments, the recombinant host cell comprises at least two copies, e.g., three, four, or five, of the polynucleotide of the present invention.

The host cell may be any cell, e.g., a prokaryotic cell or a eukaryotic cell.

The host cell may be any mammalian cell, microbial cell, or plant cell.

Preferably, the mammalian host cell is a mouse, rat, monkey, or human cell.

Preferably, the microbial cell is a prokaryotic cell (e.g., bacterial cell) or a fungal cell (e.g., a filamentous fungal cell or a yest cell).

The host cell may be any microbial or plant cell, e.g., a prokaryotic cell or a fungal cell. The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. Most preferably the bacterial host cell is a Bacillus licheniformis cell.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168:111-115), competent cell transformation (see, e.g., Young and Spizizen, 1961, J. Bacteriol. 81:823-829, or Dubnau and Davidoff-Abelson, 1971, J. Mol. Biol. 56:209-221), electroporation (see, e.g., Shigekawa and Dower, 1988, Biotechniques 6:742-751), or conjugation (see, e.g., Koehler and Thorne, 1987, J. Bacteriol. 169:5271-5278). The introduction of DNA into an E. coli cell may be effected by protoplast transformation (see, e.g., Hanahan, 1983, J. Mol. Biol. 166:557-580) or electroporation (see, e.g., Dower et al., 1988, Nucleic Acids Res. 16:6127-6145). The introduction of DNA into a Streptomyces cell may be effected by protoplast transformation, electroporation (see, e.g., Gong et al., 2004, Folia Microbiol. (Praha) 49:399-405), conjugation (see, e.g., Mazodier et al., 1989, J. Bacteriol. 171:3583-3585), or transduction (see, e.g., Burke et al., 2001, Proc. Natl. Acad. Sci. USA 98:6289-6294). The introduction of DNA into a Pseudomonas cell may be effected by electroporation (see, e.g., Choi et al., 2006, J. Microbiol. Methods 64:391-397) or conjugation (see, e.g., Pinedo and Smets, 2005, Appl. Environ. Microbiol. 71:51-57). The introduction of DNA into a Streptococcus cell may be effected by natural competence (see, e.g., Perry and Kuramitsu, 1981, Infect. Immun. 32:1295-1297), protoplast transformation (see, e.g., Catt and Jollick, 1991, Microbios 68:189-207), electroporation (see, e.g., Buckley et al., 1999, Appl. Environ. Microbiol. 65:3800-3804), or conjugation (see, e.g., Clewell, 1981, Microbiol. Rev. 45:409-436). However, any method known in the art for introducing DNA into a host cell can be used.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Pichia pastoris (also known as Khomagataella phaffii), Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell. Most preferably the yeast host cell is a Pichia pastoris cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolismis obligately aerobic. In contrast, vegetative growth by yeasts such as Saccharomyces cerevisiae is by budding of a unicellular thallus and carbon catabolismmay be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell. More preferably the filamentous fungal host cell is an Aspergillus niger, Aspergillus oryzae, or Trichoderma reesei cell. Most preferably the filamentous fungal host cell is an Aspergillus niger or Aspergillus oryzae cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81:1470-1474, and Christensen et al., 1988, Bio/Technology 6:1419-1422. Suitable methods for transforming Fusarium species are described by Malardier et al., 1989, Gene 78:147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153:163; and Hinnen et al., 1978, Proc. Natl. Acad. Sci. USA 75:1920.

### Examples

### Materials and Methods

Unless otherwise stated, DNA manipulations and transformations were performed using standard methods of molecular biology as described in Sambrook et al. (1989) Molecular cloning: A laboratory manual, Cold Spring Harbor lab., Cold Spring Harbor, NY; Ausubel, F. M. et al. (eds.) “Current protocols in Molecular Biology”, John Wiley and Sons, 1995; Harwood, C. R., and Cut-ting, S. M. (eds.) “Molecular Biological Methods for Bacillus”. John Wiley and Sons, 1990.

Purchased material (E. coli and kits)

E. coli DH5a (Toyobo) and Stellar (TakaRa) are used for plasmid construction and amplification. Amplified plasmids are recovered with Qiagen Plasmid Kit (Qiagen). Ligation is done with either NEBuilder HiFi DNA Assembly Cloning kit (New England Biolabs, Inc.) or In-Fusion kit (Clontech Laboratories, Inc.) according to the manufactory instructions. Polymerase Chain Reaction (PCR) is carried out with either PrimeSTAR GXL DNA polymerase kit (TaKaRa). QIAquick™ Gel Extraction Kit (Qiagen) is used for either the purification of PCR fragments or extraction of DNA fragment from agarose gel.

### Enzymes

Enzymes for DNA manipulations (e.g. restriction endonucleases, ligases etc.) are obtain-able from New England Biolabs, Inc. and were used according to the manufacturer's instructions.

### Plasmids

pBluescript II SK-(Stratagene #212206).

The CRISPR-Cas9 plasmids described in examples contains U6-2 promoter from Magnaporthe oryzae, Aspergillus fumigatus tRNA Gly promoter, A. fumigatus tef1 promoter and hph marker gene. Details are described in the example section.

The sequence for Po AMG harboring the amyloglucosidase from Penicillium oxalicum is described in WO2011/127802.

The pAT3530 CRISPR-ma7d-AID-UGI plasmid is described in Example 7.

### Microbial Strains

The expression host strain Aspergillus niger M1364 was isolated by Novozymes and is a derivative of Aspergillus niger NN049184 which was isolated from soil as described in Example 14 of WO 2012/160093. M1364 is a strain which produces the glucoamylase from Penicillium oxalicum (Po AMG).

The expression host strain Aspergillus oryzae JaL355 is described in Example 10 of WO 2005/070962.

Bacillus subtilis pp 3724: This strain is a donor strain for conjugation of Bacillus strains as described in WO 1996/029418.

Bacillus licheniformis SJ1904: This strain is described in WO 2008/066931.

### Medium

COVE trace metals solution was composed of 0.04 g of NaB407.10H2O, 0.4 g of CuSO4·5H2O, 1.2 g of FeSO4·7H2O, 0.7 g of MnSO4·H2O, 0.8 g of Na2Mo02.2H2O, 10 g of ZnSO4·7H2O, and deionized water to 1 liter.

50× COVE salts solution was composed of 26 g of KCl, 26 g of MgSO4·7H2O, 76 g of KH2PO4, 50 ml of COVE trace metals solution, and deionized water to 1 liter.

COVE medium was composed of 342.3 g of sucrose, 20 ml of 50× COVE salts solution, 10 ml of 1 M acetamide, 10 ml of 1.5 M CsCl2, 25 g of Noble agar, and deionized water to 1 liter. COVE-N-Gly plates were composed of 218 g of sorbitol, 10 g of glycerol, 2.02 g of KNO3, 50 ml of COVE salts solution, 25 g of Noble agar, and deionized water to 1 liter.

COVE-N(tf) was composed of 342.3 g of sucrose, 3 g of NaNO3, 20 ml of COVE salts solution, 30 g of Noble agar, and deionized water to 1 liter.

COVE-N top agarose was composed of 342.3 g of sucrose, 3 g of NaNO3, 20 ml of COVE salts solution, 10 g of low melt agarose, and deionized water to 1 liter.

COVE-N was composed of 30 g of sucrose, 3 g of NaNO3, 20 ml of COVE salts solution, 30 g of Noble agar, and deionized water to 1 liter.

STC buffer was composed of 0.8 M sorbitol, 25 mM Tris pH 8, and 25 mM CaCI2.

STPC buffer was composed of 40% PEG 4000 in STC buffer.

LB medium was composed of 10 g of tryptone, 5 g of yeast extract, 5 g of sodium chloride, and deionized water to 1 liter.

LB plus ampicilin plates were composed of 10 g of tryptone, 5 g of yeast extract, 5 g of sodium chloride, 15 g of Bacto agar, ampicillin at 100 μg per ml, and deionized water to 1 liter.

YPG medium was composed of 10 g of yeast extract, 20 g of Bacto peptone, 20 g of glucose, and deionized water to 1 liter.

SOC medium was composed of 20 g of tryptone, 5 g of yeast extract, 0.5 g of NaCl, 10 ml of 250 mM KCl, and deionized water to 1 liter.

TAE buffer was composed of 4.84 g of Tris Base, 1.14 ml of Glacial acetic acid, 2 ml of 0.5 M EDTA pH 8.0, and deionized water to 1 liter.

Bacillus strains were grown on LB agar (10 g/l Tryptone, 5 g/l yeast extract, 5 g/l NaCl, 15 g/l agar) plates, on Difco Tryptose Blood Agar Base plates, or in LB liquid medium (10 g/l Tryptone, 5 g/l yeast extract, 5 g/l NaCl).

To select for erythromycin resistance, agar media were supplemented with 1 μg/ml erythromycin and 25 μg/ml lincomycin, and liquid media were supplemented with 5 μg/ml erythromycin. To select for tetracycline resistance, agar and liquid media were supplemented with 15 μg/ml tetracycline. To select for erythromycin resistance and tetracycline resistance simultaneously, agar and liquid media were supplemented with 2 μg/ml erythromycin and 15 μg/ml tetracycline.

Spizizen I and Spizizen II media were used for preparation and transformation of competent Bacillus subtilis cells.

Spizizen I medium consists of 1× Spizizen salts (6 g/l KH2P04, 14 g/l K2HP04, 2 g/l (NH4) 2S04, 1 g/l sodium citrate dihydrate, 0.2 g/l MgS04.7H2O, pH 7.0), 0.5% glucose, 0.1% yeast extract, and 0.02% casein hydrolysate.

Spizizen II medium consists of Spizizen I medium supplemented with 0.5 mM CaCI2, and 2.5 mM MgCl2.

Conjugation donor strains were supplemented with 100 μg/ml D-alanine.

Transformation of Aspergillus niger

Transformation of Aspergillus species can be achieved using the general methods for yeast transformation. The preferred procedure for the invention is described below.

A. niger host strain was inoculated to 100 ml of YPG medium supplemented with 10 mM uridine and incubated for 16 hrs at 32° C. at 80 rpm. Pellets were collected and washed with 0.6 M KCl, and resuspended 20 ml 0.6 M KCl containing a commercial β-glucanase product (GLUCANEX™, Novozymes A/S, Bagsværd, Denmark) at a final concentration of 20 mg per ml. The suspension was incubated at 32° C. at 80 rpm until protoplasts were formed, and then washed twice with STC buffer. The protoplasts were counted with a hematometer and resuspended and adjusted in an 8:2:0.1 solution of STC: STPC: DMSO to a final concentration of 2.5×10⁷ proto-plasts/ml. Approximately 4 μg of plasmid DNA was added to 100 μl of the protoplast suspension, mixed gently, and incubated on ice for 30 minutes. One ml of SPTC was added and the protoplast suspension was incubated for 20 minutes at 37° C. After the addition of 10 ml of 50° C. Cove or Cove-N top agarose, the reaction was poured onto Cove or Cove-N(tf) agar plates and the plates were incubated at 32° C. for 5 days.

Transformation of Aspergillus oryzae

Aspergillus transformation was done as described by Christensen et al.; Biotechnology 1988 6 1419-1422. In short, A. oryzae mycelia were grown in a rich nutrient broth. The mycelia were separated from the broth by filtration. The enzyme preparation Glucanex® (Novozymes) was added to the mycelia in osmotically stabilizing buffer such as 1.2 M MgSO4 buffered to pH 5.0 with sodium phosphate. The suspension was incubated for 60 minutes at 37 degrees C. with agitation. The protoplast was filtered through mira-cloth to remove mycelial debris. The protoplast was harvested and washed twice with STC (1.2 M sorbitol, 10 mM CaCI2, 10 mM Tris-HCl pH 7.5). The protoplasts were finally re-suspended in 200-1000 microl STC.

For transformation, 1 μg CRISPR-AID-UGI plasmid (pAT3532-3537) was added to 100 μl protoplast suspension and then 200 μl PEG solution (60% PEG 4000, 10 mM CaCI2, 10 mM Tris-HCl pH 7.5) was added and the mixture is incubated for 20 minutes at room temperature. The protoplast was harvested and washed twice with 1.2 M sorbitol. The protoplast was finally re-suspended 200 μl 1.2 M sorbitol. Transformants containing the pyrG gene were selected for its ability to grow on minimal plates without addition of 10 mM uridine (Cove D. J. 1966. Biochem. Biophys. Acta. 113:51-56) containing 1.0 M sucrose as carbon source, 10 mM NaNO4 as nitrogen source. After 5-7 days of growth at 37 degrees C., stable transformants appeared as vigorously growing and sporulating colonies. Transformants were purified one through conidiophores.

### Transformation and Conjugation of Bacillus Cells

Competent cells of Bacillus subtilis strains prepared and transformed according to the method described in Yasbin et al. (1973): Transformation and transfection in lysogenic strains of Bacillus subtilis 168. J. Bacteriol. 113, 540-548.

Conjugation of Bacillus licheniformis was performed essentially as described in WO 1996/029418.

### PCR Amplifications in Examples

Polymerase Chain Reaction (PCR) was carried out with PrimeSTAR GXL DNA polymerase (TaKaRa) as follows.

| Component | Volume | | Final Concentration |
|---|---|---|---|
| 5x PrimeSTAR GXL Buffer | 10 | μl | 1x |
| 2.5 mM dNTPs | 4 | μl | 0.2 mM each |
| 5 pmol/μl Primer #1 | 2 | μl | 0.2 μM |
| 5 pmol/μl Primer #2 | 2 | μl | 0.2 μM |
| Template DNA | X | μl | |
| Genomic DNA | 10-200 ng/50 μl | | |
| or Plasmid DNA | 1-50 ng/50 μl | | |
| PCR grade water | Y | μl | |
| PrimeSTAR GXL polymerase | 1 | μl | 1.25 U/50 μl |
| (1.25 U/μl) | | | |
| Total reaction volume | 50 | μl | |

3-step cycle:

| | |
|---|---|
| Pre-denaturation: 98° C., 2 min. | |
| Denaturation: 98° C., 15 sec. | [symbol] 25-30 cycles |
| Annealing: Tm-[5-10] ° C., 15 sec. | |
| Extension: 68° C., 1 min./kb | |

For Examples 9 to 11, Polymerase Chain Reaction (PCR) was carried out with SapphireAmp Fast PCR Master Mix (Takara Bio) as follows.

| | | Expected PCR |
|---|---|---|
| Primer ID | Sequence | fragment size |
| 1202334 | TTGCACCGTCTAATGG (SEQ ID NO: 165) | 757 bp |
| 1228373 | GATGATGCCTTCCTTCAGTT (SEQ ID NO: 166) | |

| Component | Volume | | Final Concentration |
|---|---|---|---|
| 2x SapphireAmp Fast PCR Master | 10 | μl | 1x |
| Mix | | | |
| 50 pmol/μl Primer 1202334 | 0.08 | μl | 0.2 μM |
| 50 pmol/μl Primer 1228373 | 0.08 | μl | 0.2 μM |
| Culture broth | 1 | μl | |
| PCR grade water | 8.84 | μl | |
| Total reaction volume | 20 | μl | |

### Pre-denaturation: 95° C., 3 min.

30 cycles of
Denaturation: 95° C., 15 sec.
Annealing: 58° C., 15 sec.
Extension: 72° C., 15 sec.
   Final extension: 72° C., 3 min.
   PART A: Aspergillus niger.

### Example 1. Construction of the plasmids which harbors the expression cassette of dCas9-AID complex and sgRNAs targeting PKS gene

Construction of dCas9 expression plasmid

The purpose of this experiment is first to prepare the plasmid to express a catalytically inactive (= “dead”) Cas9 (i.e. dCas9 which include D10A and D840A substitutions to Cas9) in A. niger strains.

The fragments of U6-2 promoter (from Magnaporthe oryzae) fused to Aspergillus fumigatus tRNA Gly and tef1 promoter-dCas9 followed by hph selective maker were amplified by PCR with primer pairs (Table 1a) using pSMai289 plasmid DNA as template.

The CRISPR-Cas9 plasmid pSMai289 contains the sequences for the M. oryzae U6-2 promoter and terminator, A. fumigatus tRNAgly (GCC) 1-6, and Cas9. The M. oryzae U6-2 promoter and terminator were identified by searching the gene annotations of the M. grisea strain 70-15 (MG8) genome sequence database from the Joint Genome Institute (JGI). The A. fumigatus tRNAgly (GCC) 1-6 gene sequence was identified ((chr4: 3650153-3650223 (+)) by searching the gene annotations of the A. fumigatus strain 293 genome sequence database from the JGI.

The amplified fragments were assembled and ligated into the 5.5-kb Nhel fragment from pHiTe132 (a derivative of pHiTe50 which is described in WO 2015/025055) using NEBuilder HiFi DNA Assembly Cloning Kit according to the instruction, to create pHiTe277. Plasmid preparation was carried out in E. coli DH5a.

TABLE 1a
Primers for U6 promoter-tRNA, dCas9 and hph genes

| | | PCR |
|---|---|---|
| | | product |
| Fragment | Primers used (5′→3′), forward and reverse | (bp) |
| Fragment 1: from | HTJP-889: ctagaaagtataggaacttcgctagctctgctcgaggccatctg | 1850 |
| M. oryzae U6-2 | (SEQ ID NO: 1) | |
| promoter to dCas9 | HTJP-890: gttcgttccaatggccagcccgatgctatacttc (SEQ ID | |
| (D10A) | NO: 2) | |
| Fragment 2: from | HTJP-891: agtatagcatcgggctggccattggaacgaactcgg (SEQ ID | 2524 |
| dCas9 (D10A) to | NO: 3) | |
| dCas9 (D840A) | HTJP-892: gattgcgggacgatagcgtcaacatcgtagtccgacaaccg | |
| | (SEQ ID NO: 4) | |
| Fragment 3: from | HTJP-893: tcggactacgatgttgacgctatcgtcccgcaatccttcct | 1852 |
| dCas9 (D840A) to | (SEQ ID NO: 5) | |
| hph marker | HTJP-894: ggtagagtaataacgcctaggacacgcaaaacgaggtacatt | |
| | (SEQ ID NO: 6) | |
| Fragment 4: hph | HTJP-895: gtcctaggcgttattactctaccgcaagg (SEQ ID NO: 7) | 2131 |
| marker | HTJP-896: taggaacttcaatcgatctagtcctaggctacgccaggaccgag- | |
| | caagc (SEQ ID NO: 8) | |

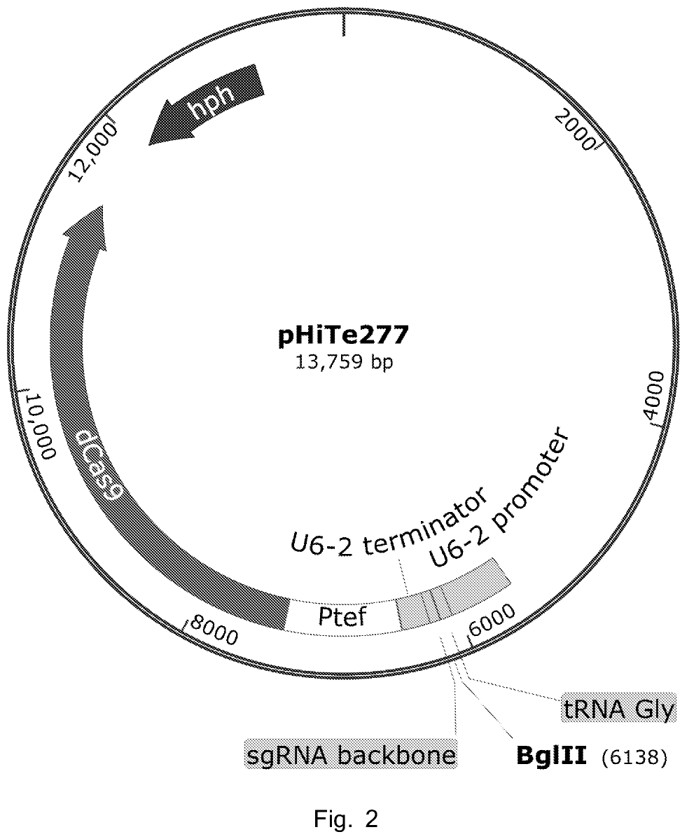

The resulting plasmid pHiTe277 contains the following elements in order (FIG. 2):
M. oryzae U6-2 promoter (SEQ ID NO:9)
A. fumigatus tRNA Gly (SEQ ID NO: 10)
Cas9 sgRNA backbone (SEQ ID NO:11)
M. oryzae U6-2 terminator (SEQ ID NO:12)
A. nidulans tef1 promoter (SEQ ID NO:13)
dCas9_coding (SEQ ID NO: 14)
dCas9_protein (SEQ ID NO: 15)
hph selection marker (SEQ ID NO:16)
   Construction of dCas9-AID expression plasmids with sgRNA expression cassettes targeting PKS gene.

The purpose of this experiment is to prepare plasmid DNAs for testing the effect of AID mutational activity when linked to dead Cas9 as a control for Mad7d-AID. The PKS (or also called wA) gene was used as a model target of dCas9-AID. The PKS knocked-out mutant would show white colored spore phenotype, which is good indicator to select for expected mutants.

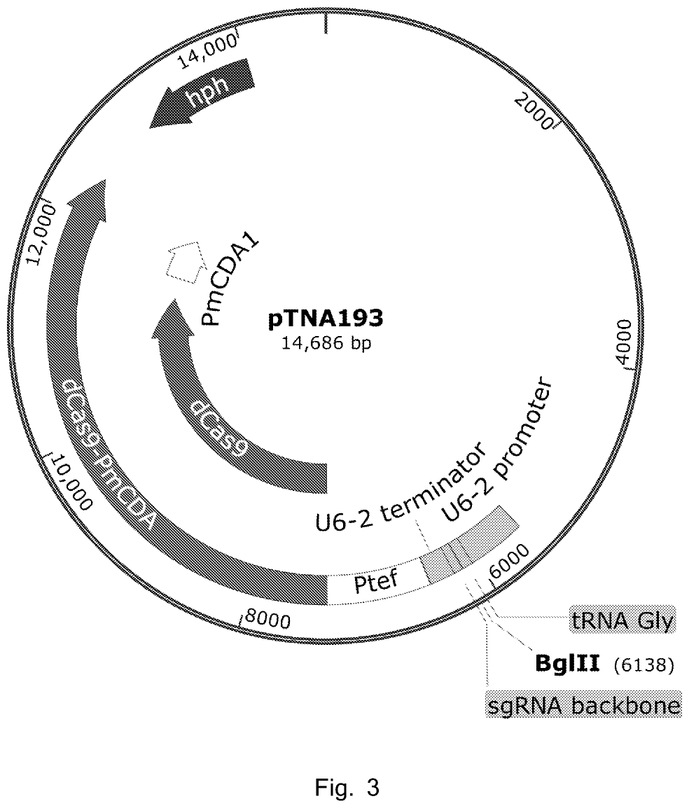

To fuse Petromyzon marinus-derived cytidine deaminase to C-terminus of dCas9 gene, codon-optimized deaminase gene was ordered as synthetic DNA (GeneArt-ThermoFisher Scientific). To preprare DNA fragments for cloning, dCas9 gene and vector region of pHite277 were independently PCR amplified. The vector fragment, dCas9 fragment and deaminase gene fragment were conjugated by NEB HiFi cloning kit, resulting in construction of pTNA 193 (dCas9-AID, sgRNA empty). The pairs of primers were described in Table 1b. FIG. 3 shows the schematic drawing of pTNA193.

TABLE 1b
Primers used for construction of dCas9-AID with
empty sgRNA vectors

| | | SEQ |
|---|---|---|
| | | ID |
| Primer name | Sequence 5′ → 3′ | NO: |
| IF_U6cas9_fwd | ttttctctgctgtctgcctcg | 17 |
| IF_dCasCDA_rev | gtcgccccccagttgactaag | 18 |
| IF_CDA3UTR_fwd | gcggacattcgatttatgccgttatg | 19 |
| IF_U63UTR_rev | agacagcagagaaaagccagatgg | 20 |
| CDAinsert_fwd | caactggggggcgacagcag | 21 |
| CDAinsert_rev | aaatcgaatgtccgcttatccggag | 22 |

Four different protospacers were designed to target the PKS gene as shown in Table 2. Pairs of oligo DNAs including each of protospacers were ordered and cloned into Bg/II site of pTNA193 by NEBuilder HiFi cloning kit, resulting in construction of pTNA197 to 200 (dCas9-AID, wA target #1 to #4, respectively). Oligo DNAs used for construction of those plasmids are shown in Table 3. To inactivate the PKS gene, protospacers were selected by following criteria: (1) stop codon (TAG, TAA, or TGA) should be introduced by C to T nucleotide conversion, (2) the target C should locate at 2nd to 5th position when numbered from PAM distal end (K. Nishida et al., (2016) Science).

TABLE 2
Protospacer sequences for disruption of PKS gene.
Potential target “C” is underlined.

| | | Protospacer |
|---|---|---|
| Plasmid | Protospacer (5′ → 3′), | adjacent |
| (target | potential target C is | motif (PAM) |
| number) | underlined | sequence |
| pTNA197 | CAGCAGTCCTCTGCTCTAGA | agg |
| (wA1) | (SEQ ID NO: 23) | |
| | sense strand | |
| pTNA198 | TCCAACCCACTCCCTGGAAT | cgg |
| (wA2) | (SEQ ID NO: 24) | |
| | sense strand | |
| pTNA199 | CCAGCATGTTGACTCGGAAT | cgg |
| (wA3) | (SEQ ID NO: 25) | |
| | antisense strand | |
| pTNA200 | TGTCCCAGCATAGTCGTCGT | agg |
| (wA4) | (SEQ ID NO: 26) | |
| | antisense strand | |

TABLE 3
The oligo DNAs for the construction of pTNA197-200. Sequence which matches
protospacers is shown in capital.

| Primer name | Sequence 5′ → 3′ | SEQ ID NO: |
|---|---|---|
| wA_sense_1 | ttcgattcacggatgatgcaCAGCAGTCCTCTGCTCTAGAgtttta- | 27 |
| | gagctagaaatagc | |
| wA_sense_1_rev | gctatttctagctctaaaacTCTAGAGCAGAGGACTGCTGtg- | 28 |
| | catcatccgtgaatcgaa | |
| wA_sense_2 | ttcgattcacggatgatgcaTCCAACCCACTCCCTGGAATgtttta- | 29 |
| | gagctagaaatagc | |
| wA_sense_2_rev | gctatttctagctctaaaacATTCCAGGGAGTGGGTT- | 30 |
| | GGAtgcatcatccgtgaatcgaa | |
| wA_anti_1 | ttcgattcacggatgatgcaCCAGCATGTTGACTCGGAATgtttta- | 31 |
| | gagctagaaatagc | |
| wA_anti_1_rev | gctatttctagctctaaaacATTCCGAGTCAACATGCTGGtg- | 32 |
| | catcatccgtgaatcgaa | |
| wA_anti_2 | ttcgattcacggatgatgcaTGTCCCAGCATAGTCGTCGTgtttta- | 33 |
| | gagctagaaatagc | |
| wA_anti_2_rev | gctatttctagctctaaaacACGACGACTATGCTGG- | 34 |
| | GACAtgcatcatccgtgaatcgaa | |

Protospacer cloning to pTNA193 was carried out as follows. The backbone plasmid pTNA 193 was digested by Bg/ll at 37° C. for 1 hour. The digested fragment was then gel purified by using QIAquick Gel Extraction kit (QIAGEN). The purified DNA fragment was mixed with each pair of oligo DNAs shown in Table 3 and protospacer sequences were cloned into the vectors by NEBuilder HiFi DNA Assembly kit. Two micro-liter of the reaction mixture were transformed into DH5a chemically competent E. coli cells. Transformants were spread onto LB plus ampicillin plates and incubated at 37° C. overnight. Plasmid DNA was purified from several transformants using a QIAGEN mini-prep kit. The plasmid DNA was screened for proper ligation by restriction enzyme digestion followed by 1.0% agarose gel electrophoresis with TAE buffer. Commercial sequence service was used to determine the actual DNA sequences.

The representative plasmid pTNA193 contains the following elements in order (FIG. 3):
M. oryzae U6-2 promoter (SEQ ID NO:9)
A. fumigatus tRNA Gly (SEQ ID NO: 10)
Cas9 sgRNA backbone (SEQ ID NO:11)
M. oryzae U6-2 terminator (SEQ ID NO: 12)
A. nidulans tef1 promoter (SEQ ID NO:13)
dCas9-AID_coding (SEQ ID NO:35)
dCas9-AID_protein (SEQ ID NO:36)
hph selection marker (SEQ ID NO: 16)

The target PKS (wA) gene sequence can also be found in the Sequence Listing (SEQ ID NO: 37).

### Example 2. Transformation of CRISPR-AID plasmids for the PKS gene inactivation

The PKS inactivation in Aspergillus niger strain M1364

The purpose of this experiment is to demonstrate that dCas9-AID can introduce target specific C to T conversion in A. niger genome, and to assess the efficiency of mutational effect. To this end, the A. niger strain M1364 was transformed with dCas9-AID plasmids targeting en-dogeneous PKS gene (described in Example 1). The dCas9-AID and sgRNA expression cassete was site-specifically integrated into A. niger genome together with a hygromycin resistance gene. After isolation and spore maturation, the spore color of transformants was checked since PKS gene inactivation should lead to white colored spore phenotype. Clones which showed the expected phenotype were further analysed by target locus sequencing.

TABLE 4
Summary of PKS-targeting dCas9-AID transformation

| | | | Colony | Iso- | White | Hit rate |
|---|---|---|---|---|---|---|
| DNA | Description | | count | lation | spore | (%) |
| pTNA193 | no sgRNA | dCas9-AID | 350 | 12 | 0 | 0% |
| pTNA197 | wA1 | dCas9-AID | 180 | 12 | 0 | 0% |
| pTNA198 | wA2 | dCas9-AID | 90 | 12 | 1 | 8% |
| pTNA199 | wA3 | dCas9-AID | 40 | 12 | 1 | 8% |
| pTNA200 | wA4 | dCas9-AID | 30 | 12 | 0 | 0% |

Table 4 shows the summary of PKS-targeting experiments. As a result, we got white spored clones at 8% efficiency by using pTNA198 or 199, which possesses wA2 or wA3 sgRNA and dCas9-AID expression cassettes, respectively. To see if the PKS gene of those white clones was mutated as expected, genome DNA was isolated and target locus was PCR-amplified. The primers are shown as follows:

For wA2 target:
SEQ ID NO: 38:
Primer pks_seq_f2: 5′ tcatatcggttctgccaagg 3′
SEQ ID NO: 39:
Primer pks_R: 5′ gttgttgacgaaagttcgcc 3′
For wA3 target:
SEQ ID NO: 40:
Primer pks_F: 5′ actgcgactgggaatctgcg 3′
SEQ ID NO: 41:
Primer pks_seq_r3: 5′ cttgtaattcttggaaatgcagg 3′

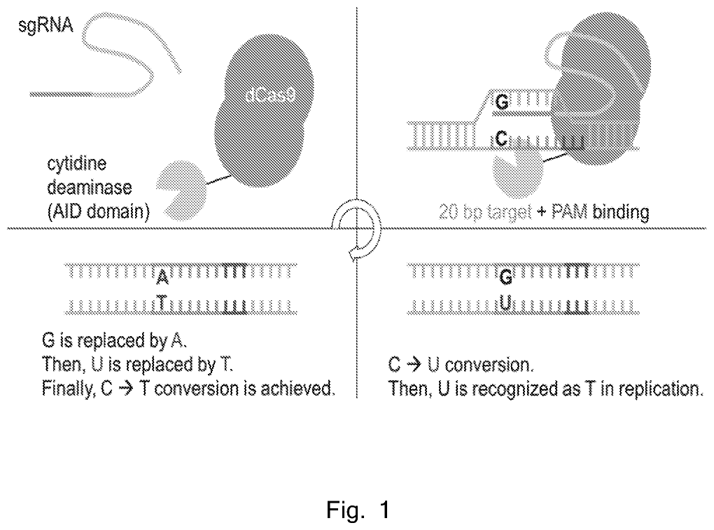

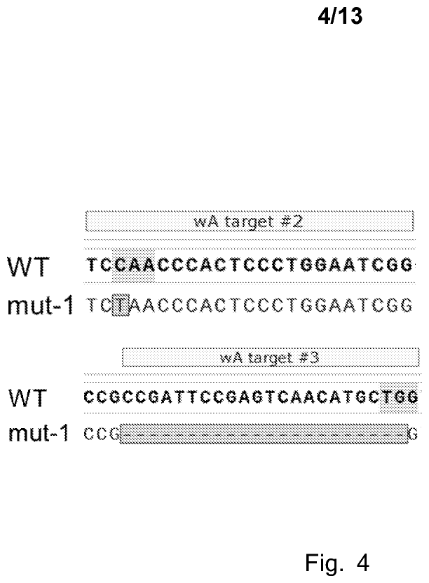

The amplicon was then gel purified by QIAquick Gel Extraction Kit (Qiagen) and sent to commercial sequence service to determine the mutation patterns at targeted region. The black spored clone which was isolated from pTNA193 (no sgRNA) treatment showed no mutation at that genomic region, whereas the white spored clones showed C to T mutation at the expected position (FIG. 4). The mutant generated by pTNA199 (wA3 target) had 17 bp deletion at the sgRNA targeted site (FIG. 4). This might be because of cellular error-prone DNA repair such as base excision repair, other than the mechanism presented in FIG. 1. Anyways, these data showed that CRISPR-Cas9-AID can work in A. niger strain.

### Example 3. Improvement of mutation efficiency by adding UGI domain

Construction of dCas9-AID-UGI expression plasmid

We showed that dCas9-AID system introduced targeted mutation at around 8% efficiency, as described in Example 2. However, this is not practical enough. Recent reports suggested that addition of UGI (uracil glycosylase inhibitor) to C-terminus of dCas9-AID improves the mutation efficiency in vivo (Rees H. A. and Liu D. R. Nature Reviews Genetics 19, 770-788 (2018)). Thus, this experiment aims to prepare plasmid DNAs which harbour dCas9-AID-UGI coding sequence and sgRNA expression cassettes. The target gene is same as what described in Example 2.

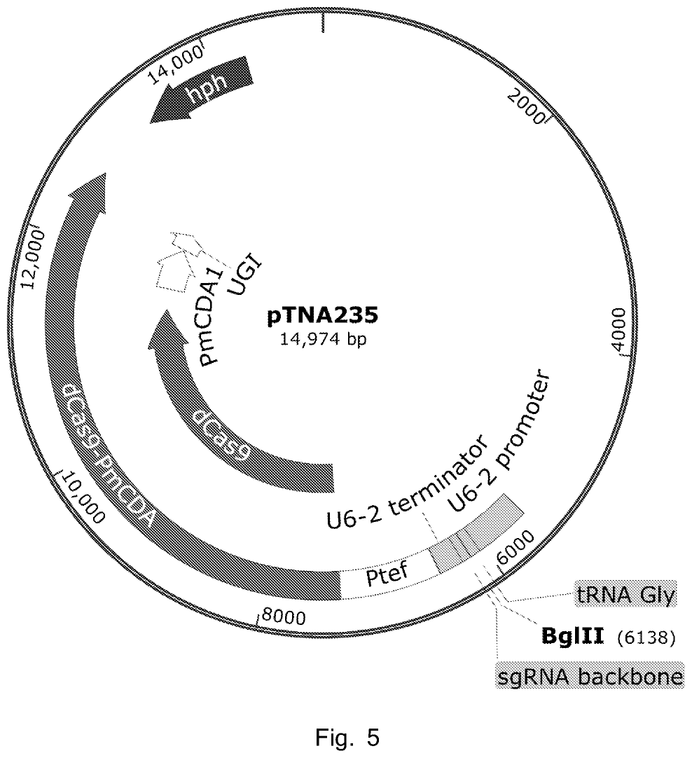

To fuse UGI gene derived from bacteriophage to C-terminus of dCas9-AID, codon-optimized UGI gene was ordered as synthetic DNA (GeneArt-ThermoFisher Scientific), see SEQ ID NO: 131. To preprare DNA fragments for cloning, pTNA193 was first digested by Xbal and Bmtl restriction enzymes and dCas9-AID encoding fragment was gel-purified. The vector part was next PCR amplified with adding BsrGI restriction site to the 5′ end of 3′UTR for dCas9-AID. This PCR fragment was then digested by BsrGI and Bmtl. Finally, UGI gene was PCR amplified with adding Xbal and BsrGI restriction sites to its 5′ and 3′ end, respectively. This UGI fragment was also digested by Xbal and BsrGI. Digested DNA fragments described above were conjugated by NEB HiFi cloning kit, resulting in construction of pTNA235 (dCas9-AID-UGI, sgRNA empty). The pairs of primers to were described in Table 5. FIG. 5 shows the schematic drawing of pTNA235.

TABLE 5
Primers used for construction of dCas9-AID-UGI with empty sgRNA vectors

| Primer name | Sequence 5′ → 3′ | Purpose | SEQ ID NO: |
|---|---|---|---|
| BsrGI-3UTR-fwd | gtctaatgtacagcggacattcgatttatgc | vector | 42 |
| | | fragment | |
| NheI-FRT-rev | agcagagctagcgaagttcctatactttctag | vector | 43 |
| | | fragment | |
| Ex-UGItop-fwd | atgatctctagaggctccggaaccaacctg | UGI | 44 |
| | | fragment | |
| UGIend-rev | aaatcgaatgtccgctgtacattag | UGI | 45 |
| | | fragment | |

The sgRNA target sequences were same as what described in Example 1. Four different target protospacers were selected (see Table 2). To target these regions by dCas9-AID-UGI, pTNA235 was digested by Bg/ll and protospacer sequences were introduced there (see Table 3), resulting in construction of pTNA240-243 (targeting wA1 to wA4, respectively).

The representative plasmid pTNA235 contains the following elements in order (FIG. 5):
M. oryzae U6-2 promoter (SEQ ID NO:9)
A. fumigatus tRNA Gly (SEQ ID NO: 10)
Cas9 sgRNA backbone (SEQ ID NO:11)
M. oryzae U6-2 terminator (SEQ ID NO: 12)
A. nidulans tef1 promoter (SEQ ID NO:13)
dCas9-AID-UGI_coding (SEQ ID NO:46)
dCas9-AID-UGI_protein (SEQ ID NO:47)
hph selection marker (SEQ ID NO:16)
   The PKS inactivation in Aspergillus niger strain M1364

The purpose of this experiment is to demonstrate the addition of UGI domain to dCas9-AID improves the mutation efficiency. To this end, as described in Example 2, the A. niger strain M1364 was transformed with pTNA235 and 240-243. After isolation and spore maturation, the spore color of transformants was checked since PKS gene inactivation should lead to white colored spore phenotype. Clones which showed the expected phenotype were further analysed by target locus sequencing.

TABLE 6
Summary of PKS-targeting dCas9-AID-UGI transformation

| | | | | | | Hit |
|---|---|---|---|---|---|---|
| | | | Colony | Iso- | White | rate |
| DNA | Description | | count | lation | spore | (%) |
| pTNA235 | no | dCas9-AID-UGI | 20 | 20 | 0 | 0% |
| | sgRNA | | | | | |
| pTNA240 | wA1 | dCas9-AID-UGI | 8 | 8 | 2 | 25% |
| pTNA241 | wA2 | dCas9-AID-UGI | 12 | 12 | 5 | 42% |
| pTNA242 | wA3 | dCas9-AID-UGI | 12 | 12 | 4 | 33% |
| pTNA243 | wA4 | dCas9-AID-UGI | 12 | 12 | 3 | 25% |

Table 6 shows the summary of PKS-targeting experiments. As a result, we got white spored clones at 25-42% efficiency, which was clearly higher than the efficiency without UGI (see Example 2). To see if the PKS gene of those white clones was mutated as expected, genome DNA was isolated and target locus was PCR-amplified. The primers are shown as follows:

For wA1 target:
SEQ ID NO: 40:
Primer pks_F: 5′ actgcgactgggaatctgcg 3′
SEQ ID NO: 48:
Primer pks_seq_r1: 5′ atttgcaagagtggtttgtg 3′
For wA2 target:
SEQ ID NO: 38:
Primer pks_seq_f2: 5′ tcatatcggttctgccaagg 3′
SEQ ID NO: 39:
Primer pks_R: 5′ gttgttgacgaaagttcgcc 3′
For wA3 target:
SEQ ID NO: 40:
Primer pks_F: 5′ actgcgactgggaatctgcg 3′
SEQ ID NO: 41:
Primer pks_seq_r3: 5′ cttgtaattcttggaaatgcagg 3′
For wA4 target:
SEQ ID NO: 40:
Primer pks_F: 5′ actgcgactgggaatctgcg 3′
SEQ ID NO: 48:
Primer pks_seq_r1: 5′ atttgcaagagtggtttgtg 3′

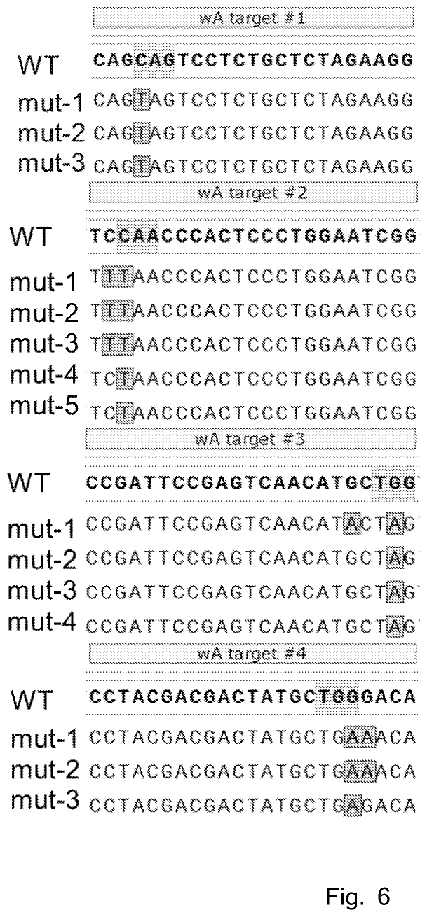

The amplicon was then gel purified by QIAquick Gel Extraction Kit (Qiagen) and sent to commercial sequence service to determine the mutation patterns at targeted region. The black spored clone which was isolated from pTNA235 (no sgRNA) treatment showed no mutation at that genomic region, whereas the white spored clones showed C to T mutation at the expected position (FIG. 6). Thus, these data showed that the addition of UGI domain to dCas9-AID actu-ally improved the mutational efficiency.

### Example 4. Construction of Mad7d

Finding putative catalytic residue of Mad7 for endonuclease activity

The catalytic site responsible for endonuclease activity in Mad7 was identified via sequence alignments with homologous sequences surrounding the active site of the otherwise distant relative FnCpf1, which can be catalytically inactivated by introducing a particular substitution (D917A) in its RuvC-like domain (Zetsche et al., 2015, Cell 163, 759-771). The corresponding conserved region was identified in Mad7 by a multiple alignment with the related Lachnospiraceae bacterium Cpf1 (LbCpf1) and Francisella tularensis Cpf1 (FnCpf1) using the MUSCLE algorithm. The alignment revealed position 877 to be relevant for the endonuclease activity of Mad7, and substitution of the native Asp with Ala (D877A) resulted in a catalytically inactive version of Mad7 (Mad7d), see the polypeptide with SEQ ID NO: 125 encoded by the polynucleotide with SEQ ID NO: 124.

The coding DNA and amino acid sequences of Mad7 nuclease can be found in SEQ ID NO: 49 and SEQ ID: 50, respectively. The amino acid sequences used for multiple alignment described above can be found in SEQ ID NO:51 to 53.

Construction of Mad7d-AID-UGI expression plasmid

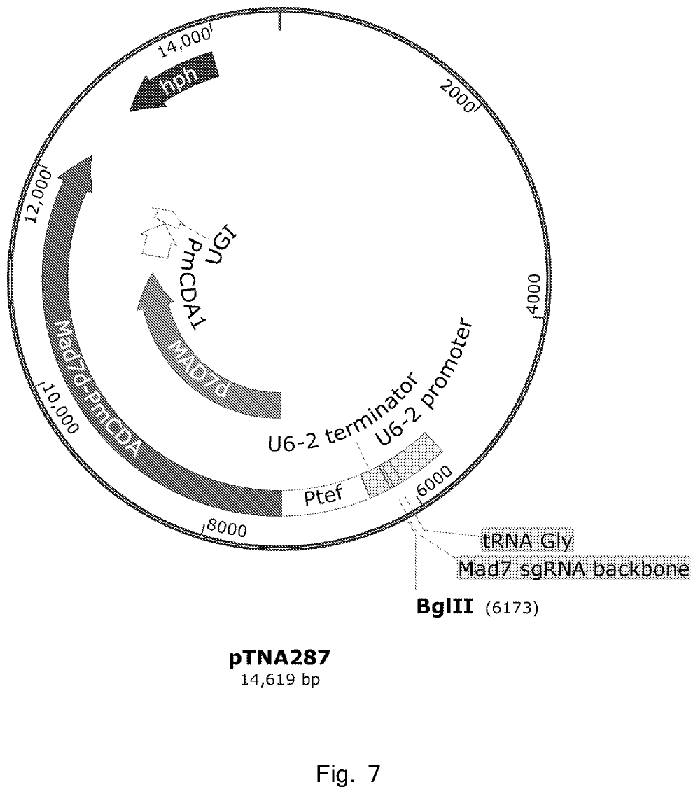

To replace dCas9 part with Mad7d in the plasmid pTNA235, Mad7d gene was ordered as synthetic DNA (GeneArt-ThermoFisher Scientific). To replace dCas part with Mad7d in pTNA235, DNA sequences on pTNA235 except for dCas9 were PCR-amplified and gel purified. Mad7d gene was cloned into that fragment by NEBuilder HiFi DNA Assembly Cloning kit (New England Biolabs, Inc.), resulting in construction of pTNA261 (Mad7d-AID-UGI, Cas9 sgRNA expression). To replace Cas9 sgRNA expression cassette with that of Mad7, pTNA261 was digested by Bg/ll and Pm/l. Mad7 sgRNA coding sequences were introduced into that site by cloning synthetic oligo DNAs with NEBuilder HiFi DNA Assembly Cloning kit, resulting in construction of pTNA287 (Mad7d-AID-UGI, Mad7 sgRNA expression (empty)). The pairs of primers to were described in Table 7. FIG. 7 shows the schematic drawing of pTNA287.

TABLE 7
Primers used for construction of Mad7d-AID-UGI with empty sgRNA vectors

| Primer name | Sequence 5′ → 3′ | SEQ ID NO: |
|---|---|---|
| IF-Ptef-rev | ggtgaaggttgtgttatgttttgtgg | 54 |
| IF-nls-fwd | agcagggctgaccccaagaag | 55 |
| IF-PtfMd7-fwd | aacacaaccttcaccatgaacaacggcacaaacaac | 56 |
| IF-nlsMd7-rev | ggggtcagccctgctaggcaagtacctcttgttctg | 57 |
| sg-site_fwd | ttcgattcacggatgatgcagtcaaaagacctttttaatttctactcttgtagatagatc | 58 |
| | ttttttttggctcttgggttcgaactgc | |
| sg-site_rev | tttggcttcggaaagcacacgtgaagggtaggaacaaagatggaatgattggca | 59 |
| | ggggtgacccaaatggtggggcataaaaaaaaagatgaccaaaacatgggc | |
| | cttgggcagttcgaacccaagagcc | |

The representative plasmid pTNA287 contains the following elements in order:
M. oryzae U6-2 promoter (SEQ ID NO:9)
A. fumigatus tRNA Gly (SEQ ID NO: 10)
Mad7 sgRNA backbone (SEQ ID NO:60)
M. oryzae U6-2 terminator (SEQ ID NO:12)
A. nidulans tef1 promoter (SEQ ID NO:13)
Mad7d-AID-UGI_coding (SEQ ID NO:61)
Mad7d-AID-UGI_protein (SEQ ID NO:62)
hph selection marker (SEQ ID NO:16)

### Example 5. Transformation of Mad7d-AID plasmids for the PKS gene inactivation

The PKS inactivation by Mad7d-AID in A. niger strain M1364

The purpose of this experiment is to demonstrate that Mad7d-AID can introduce target specific C to T conversion in A. niger genome, and to assess the efficiency of mutational effect, as shown in Example 2 for the case of dCas9-AID. Since nobody has reported the activity of Mad7d linked deaminase, we first aimed to clarify the target position of cytidine as a substrate for AID. To this end, 19 target sequences with varied target positions on PKS locus were designed (Table 8), each of which would form premature stop codon when C to T mutation is introduced by Mad7d-AID. Target position of C is numbered from PAM proximal end. These protospacer sequences were cloned into Bg/ll digested site of pTNA287, resulting in construction of pTNA296-307 and pTNA324-330 using the oligo DNAs shown in Table 9.

TABLE 8
Protospacer sequences for disruption of PKS gene.
Potential target “C” is underlined.

| | | Protospacer |
|---|---|---|
| Plasmid | Protospacer (5′ → 3′), | adjacent motif |
| (target | potential target C | (PAM) sequence |
| number) | is underlined | (5′ → 3′) |
| pTNA296 | TTGGAGACCAGACCAGCGACA | cttt |
| MdwA1 | (SEQ ID NO: 63) | |
| | sense strand | |
| pTNA297 | GAGACCAGACCAGCGACATCG | tttg |
| MdwA2 | (SEQ ID NO: 64) | |
| | sense strand | |
| pTNA298 | TTCCAGCAATGCTTCCATGCA | cttt |
| MdwA3 | (SEQ ID NO: 65) | |
| | sense strand | |
| pTNA299 | CAGCAATGCTTCCATGCAATT | tttc |
| MdwA4 | (SEQ ID NO: 66) | |
| | sense strand | |
| pTNA300 | CATGCAATTCGTCAAGAGATC | cttc |
| MdwA5 | (SEQ ID NO: 67) | |
| | sense strand | |
| pTNA301 | TCGAGTTCGAGAACTGGTGGA | tttt |
| MdwA6 | (SEQ ID NO: 68) | |
| | sense strand | |
| pTNA302 | CGATCTCGAGTAGCGCCACTC | tttc |
| MdwA7 | (SEQ ID NO: 69) | |
| | sense strand | |
| pTNA303 | ATCCCTGGCGGTAACCGAGCA | tttc |
| MdwA8 | (SEQ ID NO: 70) | |
| | sense strand | |
| pTNA304 | AGGAAAGCGGCGAACTTTCGT | cttc |
| MdwA9 | (SEQ ID NO: 71) | |
| | sense strand | |
| pTNA305 | ACTGCATCGCAAGAAGCCAGG | tttg |
| MdwA10 | (SEQ ID NO: 72) | |
| | sense strand | |
| pTNA306 | GCCCTGTCGTGGTACAAGTGG | ctta |
| MdwA11 | (SEQ ID NO: 73) | |
| | sense strand | |
| pTNA307 | TGGCCGGCGTGCTCAGTTGCT | tttg |
| MdwA12 | (SEQ ID NO: 74) | |
| | sense strand | |
| pTNA324 | TACAGGAGGTTCCAGAAGCTC | tttg |
| MdwA13 | (SEQ ID NO: 75) | |
| | antisense strand | |
| pTNA325 | TCCAGATCGAATTGCAAGCCA | tttc |
| MdwA14 | (SEQ ID NO: 76) | |
| | antisense strand | |
| pTNA326 | CTGAAGCTGGCCGATTCCAGG | tttc |
| MdwA15 | (SEQ ID NO: 77) | |
| | antisense strand | |
| pTNA327 | ACACCCAGAGCAGTCCAGTAA | cttc |
| MdwA16 | (SEQ ID NO: 78) | |
| | antisense strand | |
| pTNA328 | GGGCCGAGACACTCCCAGTTA | cttg |
| MdwA17 | (SEQ ID NO: 79) | |
| | antisense strand | |
| pTNA329 | AGATCCCACTTGTAGGCTGGG | cttg |
| MdwA18 | (SEQ ID NO: 80) | |
| | antisense strand | |
| pTNA330 | TTCTCCGCCCAATCCGCTGTC | cttc |
| MdwA19 | (SEQ ID NO: 81) | |
| | antisense strand | |

TABLE 9
The oligo DNAs for the construction of pTNA296-307 and 324-330. Sequence which
matches protospacers is shown in capital.

| Primer | | |
|---|---|---|
| name | Sequence 5′ → 3′ | SEQ ID NO: |
| 296-C9- | taatttctactcttgtagatTTGGAGACCAGACCAGCGACAtttttttggctcttggg | 82 |
| sense | ttc | |
| 296-C9- | gaacccaagagccaaaaaaaTGTCGCTGGTCTGGTCTCCAAatctacaag | 83 |
| anti | agtagaaatta | |
| 297-C6- | taatttctactcttgtagatGAGACCAGACCAGCGACATCGtttttttggctcttggg | 84 |
| sense | ttc | |
| 297-C6- | gaacccaagagccaaaaaaaCGATGTCGCTGGTCTGGTCTCatctacaa | 85 |
| anti | gagtagaaatta | |
| 298- | taatttctactcttgtagatTTCCAGCAATGCTTCCATGCAtttttttggctcttgggtt | 86 |
| C4_7- | c | |
| sense | | |
| 298- | gaacccaagagccaaaaaaaTGCATGGAAGCATTGCTGGAAatctacaa | 87 |
| C4_7- | gagtagaaatta | |
| anti | | |
| 299- | taatttctactcttgtagatCAGCAATGCTTCCATGCAATTtttttttggctcttgggtt | 88 |
| C1_4- | c | |
| sense | | |
| 299- | gaacccaagagccaaaaaaaAATTGCATGGAAGCATTGCTGatctacaag | 89 |
| C1_4- | agtagaaatta | |
| anti | | |
| 300-C13- | taatttctactcttgtagatCATGCAATTCGTCAAGAGATCtttttttggctcttgggtt | 90 |
| sense | c | |
| 300-C13- | gaacccaagagccaaaaaaaGATCTCTTGACGAATTGCATGatctacaag | 91 |
| anti | agtagaaatta | |
| 301- | taatttctactcttgtagatTCGAGTTCGAGAACTGGTGGAtttttttggctcttgggt | 92 |
| C2_8- | tc | |
| sense | | |
| 301- | gaacccaagagccaaaaaaaTCCACCAGTTCTCGAACTCGAatctacaag | 93 |
| C2_8- | agtagaaatta | |
| anti | | |
| 302-C21- | taatttctactcttgtagatCGATCTCGAGTAGCGCCACTCtttttttggctcttgggt | 94 |
| sense | tc | |
| 302-C21- | gaacccaagagccaaaaaaaGAGTGGCGCTACTCGAGATCGatctacaa | 95 |
| anti | gagtagaaatta | |
| 303-C16- | taatttctactcttgtagatATCCCTGGCGGTAACCGAGCAtttttttggctcttggg | 96 |
| sense | ttc | |
| 303-C16- | gaacccaagagccaaaaaaaTGCTCGGTTACCGCCAGGGATatctacaa | 97 |
| anti | gagtagaaatta | |
| 304-C11- | taatttctactcttgtagatAGGAAAGCGGCGAACTTTCGTtttttttggctcttgggt | 98 |
| sense | tc | |
| 304-C11- | gaacccaagagccaaaaaaaACGAAAGTTCGCCGCTTTCCTatctacaag | 99 |
| anti | agtagaaatta | |
| 305-C18- | taatttctactcttgtagatACTGCATCGCAAGAAGCCAGGtttttttggctcttggg | 100 |
| sense | ttc | |
| 305-C18- | gaacccaagagccaaaaaaaCCTGGCTTCTTGCGATGCAGTatctacaag | 101 |
| anti | agtagaaatta | |
| 306-C15- | taatttctactcttgtagatGCCCTGTCGTGGTACAAGTGGtttttttggctcttggg | 102 |
| sense | ttc | |
| 306-C15- | gaacccaagagccaaaaaaaCCACTTGTACCACGACAGGGCatctacaa | 103 |
| anti | gagtagaaatta | |
| 307-C14- | taatttctactcttgtagatTGGCCGGCGTGCTCAGTTGCTtttttttggctcttggg | 104 |
| sense | ttc | |
| 307-C14- | gaacccaagagccaaaaaaaAGCAACTGAGCACGCCGGCCAatctacaa | 105 |
| anti | gagtagaaatta | |
| 324- | taatttctactcttgtagatTACAGGAGGTTCCAGAAGCTCtttttttggctcttgggt | 106 |
| C12,13- | tc | |
| sense | | |
| 324- | gaacccaagagccaaaaaaaGAGCTTCTGGAACCTCCTGTAatctacaag | 107 |
| C12,13- | agtagaaatta | |
| anti | | |
| 325- | taatttctactcttgtagatTCCAGATCGAATTGCAAGCCAtttttttggctcttgggt | 108 |
| C2,3- | tc | |
| sense | | |
| 325- | gaacccaagagccaaaaaaaTGGCTTGCAATTCGATCTGGAatctacaag | 109 |
| C2, 3-anti | agtagaaatta | |
| 326- | taatttctactcttgtagatCTGAAGCTGGCCGATTCCAGGtttttttggctcttggg | 110 |
| C17, 18- | ttc | |
| sense | | |
| 326- | gaacccaagagccaaaaaaaCCTGGAATCGGCCAGCTTCAGatctacaa | 111 |
| C17, 18- | gagtagaaatta | |
| anti | | |
| 327- | taatttctactcttgtagatACACCCAGAGCAGTCCAGTAAtttttttggctcttgggt | 112 |
| C15, 16- | tc | |
| sense | | |
| 327- | gaacccaagagccaaaaaaaTTACTGGACTGCTCTGGGTGTatctacaag | 113 |
| C15, 16- | agtagaaatta | |
| anti | | |
| 328- | taatttctactcttgtagatGGGCCGAGACACTCCCAGTTAtttttttggctcttggg | 114 |
| C15, 16- | ttc | |
| 2-sense | | |
| 328- | gaacccaagagccaaaaaaaTAACTGGGAGTGTCTCGGCCCatctacaa | 115 |
| C15, 16- | gagtagaaatta | |
| 2-anti | | |
| 329- | taatttctactcttgtagatAGATCCCACTTGTAGGCTGGGtttttttggctcttgggt | 116 |
| C6,7- | tc | |
| sense | | |
| 329- | gaacccaagagccaaaaaaaCCCAGCCTACAAGTGGGATCTatctacaa | 117 |
| C6, 7-anti | gagtagaaatta | |
| 330- | taatttctactcttgtagatTTCTCCGCCCAATCCGCTGTCtttttttggctcttgggt | 118 |
| C9, 10- | tc | |
| sense | | |
| 330- | gaacccaagagccaaaaaaaGACAGCGGATTGGGCGGAGAAatctacaa | 119 |
| C9, 10- | gagtagaaatta | |
| anti | | |

Next, as described in Example 2, the A. niger strain M1364 was transformed those plasmid DNAs. The Mad7d-AID and sgRNA expression cassette was site-specifically integrated into A. niger genome together with a hygromycin resistance gene. After isolation and spore maturation, the spore color of transformants was checked. Clones which showed the expected phenotype (white spore colour) were further analysed by target locus sequencing.

TABLE 10
Summary of PKS-targeting Mad7d-AID transformation

| | | | Colony | | White | Hit rate |
|---|---|---|---|---|---|---|
| DNA | Description | | count | Isolation | spore | (%) |
| pTNA287 | no sgRNA | Mad7d-AID-UGI | >200 | 12 | 0 | 0% |
| pTNA296 | MdwA1 | Mad7d-AID-UGI | >200 | 12 | 0 | 0% |
| pTNA297 | MdwA2 | Mad7d-AID-UGI | >200 | 12 | 0 | 0% |
| pTNA298 | MdwA3 | Mad7d-AID-UGI | >200 | 12 | 0 | 0% |
| pTNA299 | MdwA4 | Mad7d-AID-UGI | >200 | 12 | 0 | 0% |
| pTNA300 | MdwA5 | Mad7d-AID-UGI | >200 | 12 | 0 | 0% |
| pTNA301 | MdwA6 | Mad7d-AID-UGI | >200 | 12 | 0 | 0% |
| pTNA302 | MdwA7 | Mad7d-AID-UGI | >200 | 12 | 0 | 0% |
| pTNA303 | MdwA8 | Mad7d-AID-UGI | >200 | 12 | 2 | 17% |
| pTNA304 | MdwA9 | Mad7d-AID-UGI | >200 | 12 | 0 | 0% |
| pTNA305 | MdwA10 | Mad7d-AID-UGI | >200 | 12 | 0 | 0% |
| pTNA306 | MdwA11 | Mad7d-AID-UGI | >200 | 12 | 0 | 0% |
| pTNA307 | MdwA12 | Mad7d-AID-UGI | >200 | 12 | 0 | 0% |
| pTNA324 | MdwA13 | Mad7d-AID-UGI | >200 | 12 | 1 | 8% |
| pTNA325 | MdwA14 | Mad7d-AID-UGI | >200 | 12 | 0 | 0% |
| pTNA326 | MdwA15 | Mad7d-AID-UGI | >200 | 12 | 0 | 0% |
| pTNA327 | MdwA16 | Mad7d-AID-UGI | >200 | 12 | 0 | 0% |
| pTNA328 | MdwA17 | Mad7d-AID-UGI | >200 | 12 | 0 | 0% |
| pTNA329 | MdwA18 | Mad7d-AID-UGI | >200 | 12 | 0 | 0% |
| pTNA330 | MdwA19 | Mad7d-AID-UGI | >200 | 12 | 0 | 0% |

As shown in Table 10, white spore mutants were obtained at 8 to 17% efficiency with effective sgRNAs. To see if the PKS gene of those white clones was mutated as expected, genome DNA was isolated and target locus was PCR-amplified. The primers are shown as follows:

For MdwA8 target:
SEQ ID NO: 120:
Primer pks_seq_f5: 5′ ttcttcaacatgtcgcctcgg 3′
SEQ ID NO: 121:
Primer pks_seq_r6: 5′ gtgttacagttgccagtgg 3′
For MdwA13 target:
SEQ ID NO: 122:
Primer pks_seq_f4: 5′ ggtacttgatgaattcgtcg 3′
SEQ ID NO: 121:
Primer pks_seq_r6: 5′ gtgttacagttgccagtgg 3′

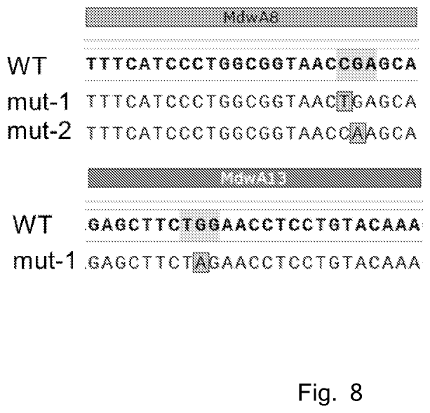

The amplicon was then gel purified by QIAquick Gel Extraction Kit (Qiagen) and sequenced to determine the mutation patterns at targeted region. The black spored clone which was isolated from pTNA287 (no sgRNA) treatment showed no mutation at read genomic region, whereas mutants had mutations at C16, G17 (antisense) by pTNA303, and C13 by pTNA324 as expected (FIG. 8). This suggests that the primary target window of Mad7d-AID is C13 to C17, and that Mad7d-AID system can introduce C to T mutations in industrially important microbes such as A. niger.

### Example 6. Investigation of optimum temperature for Mad7d-AID activity

The PKS inactivation by Mad7d-AID with various incubation temperatures

The purpose of this experiment is to investigate the optimum temperature for Mad7d-AID activity. To this end, pTNA303 was transformed into M1364 host strain, and transformants were incubated at 25, 30, or 34° C. until spore maturation. The procedure described in Example 5 was followed in this experiment except for incubation temperature. The results are summarized in Table 11.

TABLE 11
Summary of pTNA303 transformation at various temperatures

| | | Incubation | Colony | Iso- | White | Hit rate |
|---|---|---|---|---|---|---|
| DNA | sgRNA | temperature | count | lation | spore | (%) |
| pTNA303 | MdwA8 | 34° C. | 50 | 12 | 8 | 67% |
| pTNA303 | MdwA8 | 30° C. | 120 | 12 | 2 | 17% |
| pTNA303 | MdwA8 | 25° C. | 220 | 12 | 0 | 0% |

As shown in Table 11, white spore mutants were obtained at 0 to 67% efficiency with a clear trend that higher temperature gives better efficiency. To see if the PKS gene of those white clones was mutated as expected, genome DNA was isolated and target locus was PCR-amplified. The primers are shown as follows:

SEQ ID NO: 120:
Primer pks_seq_f5: 5′ ttcttcaacatgtcgcctcgg 3′
SEQ ID NO: 121:
Primer pks_seq_r6: 5′ gtgttacagttgccagtgg 3′

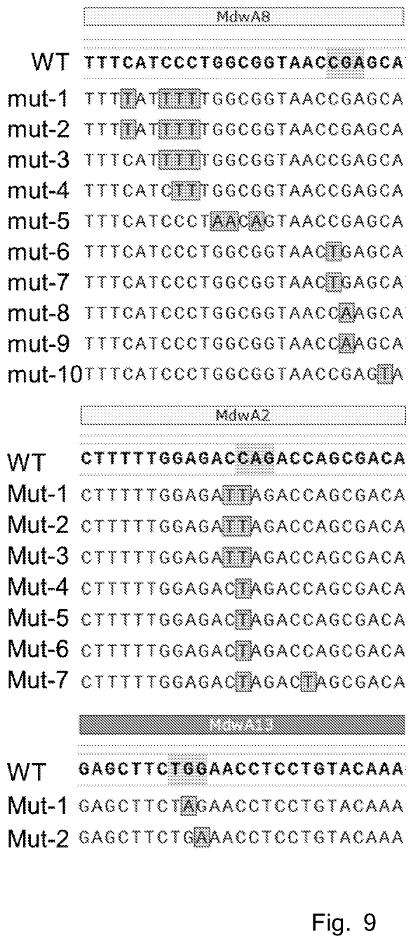

The amplicon was then gel purified by QIAquick Gel Extraction Kit (Qiagen) and sent to commercial sequence service to determine the mutation patterns at targeted region. Interestingly, the white spore mutants had C to T or G to A mutations across sgRNA targeted sequences, as shown in FIG. 9. G to A mutations may be introduced by antisense C to T mutations.

Next, to see if this efficiency improvement is general to other sgRNAs or not, we tested 13 sgRNAs for 34° C. incubation, as listed in Table 12.

TABLE 12
Summary of re-visiting PKS-targeting sgRNAs for 34° C. incubation

| | | | Colony | | White | Hit rate |
|---|---|---|---|---|---|---|
| DNA | Description | | count | Isolation | spore | (%) |
| pTNA287 | no sgRNA | Mad7d-AID-UGI | 50-100 | 12 | 0 | 0% |
| pTNA296 | MdwA1 | Mad7d-AID-UGI | 50-100 | 12 | 1 | 8% |
| pTNA297 | MdwA2 | Mad7d-AID-UGI | 50-100 | 12 | 7 | 58% |
| pTNA298 | MdwA3 | Mad7d-AID-UGI | 50-100 | 12 | 0 | 0% |
| pTNA299 | MdwA4 | Mad7d-AID-UGI | 50-100 | 12 | 1 | 8% |
| pTNA300 | MdwA5 | Mad7d-AID-UGI | 50-100 | 12 | 1 | 8% |
| pTNA301 | MdwA6 | Mad7d-AID-UGI | 50-100 | 12 | 1 | 8% |
| pTNA302 | MdwA7 | Mad7d-AID-UGI | 50-100 | 12 | 0 | 0% |
| pTNA303 | MdwA8 | Mad7d-AID-UGI | 50-100 | 12 | 7 | 58% |
| pTNA304 | MdwA9 | Mad7d-AID-UGI | 50-100 | 12 | 0 | 0% |
| pTNA324 | MdwA13 | Mad7d-AID-UGI | 50-100 | 12 | 2 | 17% |
| pTNA325 | MdwA14 | Mad7d-AID-UGI | 50-100 | 12 | 3 | 25% |
| pTNA326 | MdwA15 | Mad7d-AID-UGI | 50-100 | 12 | 0 | 0% |
| pTNA327 | MdwA16 | Mad7d-AID-UGI | 50-100 | 12 | 0 | 0% |

As shown in Table 12, white spore mutants were obtained at 0 to 58% efficiency. When compared to Table 10, editing efficiency was clearly improved with some of sgRNAs like MdwA2, MdwA8, MdwA13, and MdwA14. To see if the PKS gene of those white clones was mutated as expected, genome DNA was isolated and target locus was PCR-amplified. The primers are shown as follows:

For MdwA1-6:
SEQ ID NO: 123:
Primer MS-test-wA3: 5′ tgaattcaactctttacaatcg 3′
SEQ ID NO: 41:
Primer pks_seq_r3: 5′ cttgtaattcttggaaatgcagg 3′
For MdwA8-14:
SEQ ID NO: 122:
Primer pks_seq_f4: 5′ ggtacttgatgaattcgtcg 3′
SEQ ID NO: 121:
Primer pks_seq_r6: 5′ gtgttacagttgccagtgg 3′

The amplicon was then gel purified by QIAquick Gel Extraction Kit (Qiagen) sequenced to determine the mutation patterns at targeted region. Some representative genotypes are also shown in FIG. 9.

These data suggest that Mad7d-AID provides at least two beneficial effects:
1) Mad7d-AID may be able to target both sense and antisense strands simultaneously. In general, sgRNA should form tight heteroduplex with targeting strand of the genome DNA due to its complementarity, which makes non-targeting strand free and targetable by AID, vice versa, targeting strand cannot be targeted because of duplex formation. As described in Example 3, dCas9-AID introduced mutations to either sense or antisense strand dependent on sgRNA targeting strand. But, as shown in FIG. 9, pTNA303 transformants had C to T and/or G to A mutations at the protospacer region despite targeting only sense strand by MdwA8 sgRNA. Although the mechanism is not yet completely clear, this effect seems to be mediated by Mad7d-AID deamination of both strands.
2) Mad7d-AID may be able to target cytidines at any position of protospacer region. as shown in FIG. 9, pTNA303 transformants had C to T and/or G to A mutations across the protospacer region (21 bp-long), which is well distinct from dCad9-AID whose target range is limited to 4 bp window within protospacer (20 bp-long), as described in Example 3. This is clearly beneficial when making mutant library, since Mad7d-AID creates larger variety of mutations from a single sgRNA than dCas9-AID.

Taken together, the Mad7d-AID system has beneficial and hitherto undescribed features especially when creating mutant library for screening purposes, which is key for development of industrially important strains.

PART B: Aspergillus oryzae.

### Example 7. Construction of pAT3530 Mad7d-AID-UGI expression plasmid for A. oryzae

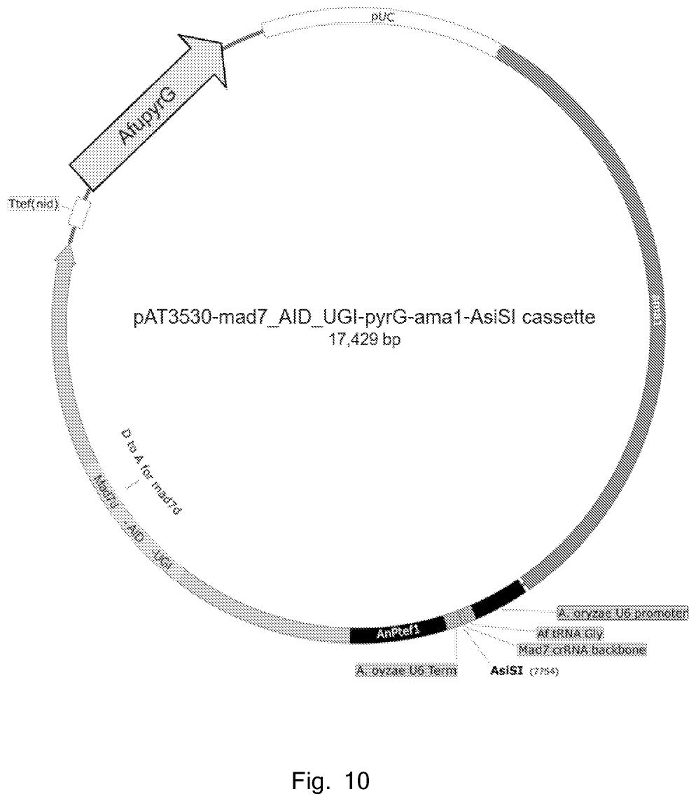

Plasmid pAT3530 (FIG. 10, SEQ ID NO: 133) is a vector ready for cloning of protospacer sequences into the single AsiSI restriction site.

Plasmid pAT3530 contains the following elements:

| Position | |
|---|---|
| in SEQ | |
| ID NO: 133 | Element |
| 15.845-1.343 | pUC plasmid |
| 1.359-1.358 | Linker sequence |
| 1.359-7.079 | Ama1 sequence for replication in A. oryzae |
| 7.080-7.115 | Linker sequence |
| 7.116-7.623 | A. oryzae U6 promoter |
| 7.624-7.714 | A. fumigatus tRNA (Gly) |
| 7.715-7.749 | Mad7 crRNA |
| 7.750-7.757 | AsiSl restriction site (used for cloning of protoscers) |
| 7.758-7.895 | A. oryzae U6 terminator |
| 7.896-7.903 | Linker sequence |
| 7.904-8.789 | A. nidulans Tef1 promoter |
| 8.790-13.817 | Mad7d-AID-UGI gene (taken from pTNA287 in |
| | Example 4) |
| 13.818-13.823 | Linker sequence |
| 13.824-14.228 | A. nidulans Tef1 terminator |
| 14.229-14.405 | Linker sequence |
| 14.406-15.844 | A. fumigatus pyrG gene |

The A. oryzae target wA gene sequence can be found in the Sequence Listing as SEQ ID NO: 134.

### Example 8. Transformation of Mad7d-AID-UGI plasmids for the wA gene inactivation in A. oryzae strain JaL355

The purpose of this experiment is to demonstrate that Mad7d-AID-UGI can introduce target specific C to T conversion in the A. oryzae genome, and to assess the efficiency of mutational effect, as shown in Example 5 for the case of Mad7d-AID. To our knowledge, nobody has reported the activity of Mad7d-linked deaminase, so we first aimed to clarify the target position of cytidine as a substrate for AID. To this end, six target sequences with varied target positions on PKS locus were designed (Table 13), each of which would form premature stop codon when C to T mutation is introduced by Mad7d-AID. Target position of C is numbered from PAM proximal end. These protospacer sequences (Table 14) were cloned into AsiSI digested site of pAT3530, resulting in construction of pAT3532-pAT3537.

TABLE 13
Protospacer sequences for disruption of wA gene in
A. oryzae. Potential target “C” is underlined.

| | | Protospacer |
|---|---|---|
| Plasmid | Protospacer (5′ → 3′), | adjacent motif |
| (target | potential target C is | (PAM) sequence |
| number) | underlined | (5′ → 3′) |
| pAT3532 | ACGCTTTGCGCCAAGAGATC | TTTC |
| | (SEQ ID NO: 135) | |
| | sense strand | |
| pAT3533 | CGCCAAGAGATCGCGAGGCT | TTTC |
| | (SEQ ID NO: 136) | |
| | sense strand | |
| pAT3534 | AAGCACTGCGACACAAAGCT | TTTC |
| | (SEQ ID NO: 137) | |
| | sense strand | |
| pAT3535 | CATTCTGCCCAAGTTACTCC | TTTC |
| | (SEQ ID NO: 138) | |
| | sense strand | |
| pAT3536 | CGGAAGCCGGTCTGTTCCAA | TTTC |
| | (SEQ ID NO: 139) | |
| | antisense strand | |
| pAT3537 | TACCTAGTGAACCCCAATAG | TTTA |
| | (SEQ ID NO: 140) | |
| | antisense strand | |

TABLE 14
The oligo DNAs for the construction of pAT3532-3537. Sequence which matches
protospacers is shown in capital.

| Primer | | |
|---|---|---|
| name | Sequence 5′ → 3′ | SEQ ID NO: |
| oAT3941 | aatttctactcttgtagatACGCTTTGCGCCAAGAGATCtttttttttgagcatttat | 141 |
| | cagc | |
| oAT3942 | aatttctactcttgtagatCGCCAAGAGATCGCGAGGCTtttttttttgagcattta | 142 |
| | tcagc | |
| oAT3943 | aatttctactcttgtagatAAGCACTGCGACACAAAGCTtttttttttgagcatttat | 143 |
| | cagc | |
| oAT3944 | aatttctactcttgtagatCATTCTGCCCAAGTTACTCCtttttttttgagcatttatc | 144 |
| | agc | |
| oAT3945 | aatttctactcttgtagatCGGAAGCCGGTCTGTTCCAAtttttttttgagcatttat | 145 |
| | cagc | |
| oAT3946 | aatttctactcttgtagatTACCTAGTGAACCCCAATAGtttttttttgagcatttatc | 146 |
| | agc | |

Next, the plasmids pAT3530 and pAT3532-pAT3537, was transformed into A. oryzae strain JaL355 as described in methods for A. oryzae transformation. The plasmids containing the Mad7d-AID-UGI and sgRNA expression cassettes was maintained as replicative plasmid by the pyrG gene on the plasmids. Thereafter all colonies having white spore colour and a corresponding number of green spore colour colonies from each transformation was spore isolated on non-selective plates (containing uridine) so the replicative plasmids were lost. All re-isolated transformants were further analysed by target locus sequencing.

TABLE 15
Summary of wA-targeting Mad7d-AID-
UGI A. oryzae transformation

| | | | | White | Green |
|---|---|---|---|---|---|
| | | | Colony | spore | spore |
| DNA | Description | | count | isolated | isolated |
| pAT3530 | no sgRNA | Mad7d-AID-UGI | >200 | 0 | 0 |
| pAT3532 | sgRNA | Mad7d-AID-UGI | >200 | 1 | 1 |
| pAT3533 | sgRNA | Mad7d-AID-UGI | >200 | 3 | 3 |
| pAT3534 | sgRNA | Mad7d-AID-UGI | >200 | 1 | 1 |
| pAT3535 | sgRNA | Mad7d-AID-UGI | >200 | 1 | 1 |
| pAT3536 | sgRNA | Mad7d-AID-UGI | >200 | 5 | 5 |
| pAT3537 | sgRNA | Mad7d-AID-UGI | >200 | 3 | 3 |

As shown in Table 15, white spore mutants were obtained with effective sgRNAs. To see if the PKS gene of those white clones was mutated as expected, genome DNA was isolated and target locus was PCR-amplified as following for the 6 targets:

For pAT3532 and pAT3533 target: PCR amplification was done with oAT3912: 5′ TCCAAGTTCTTTGCATGC 3′ (SEQ ID NO: 147) and oAT3613: 5′ TATCTCAGGTTAGGCTCG 3′ (SEQ ID NO: 148) resulting in an amplicon on 500 bp.

For pAT3534 target: PCR amplification was done with oJaL 188: 5′ CCATGGTCCTTAC-CATGC 3′ (SEQ ID NO: 149) and oAT3616 5′ TATTTATCTCCCGATAGTCATC 3′ (SEQ ID NO: 150) resulting in an amplicon on 812 bp.

For pAT3535 amplification target: PCR was done with oAT919: 5′ CTGGCTGTCAAGGCTTCC 3′ (SEQ ID NO: 151) and oAT1040: 5′ TTTGTGGTGCAGCTTGAAT 3′ (SEQ ID NO: 152) resulting in an amplicon on 733 bp.

For pAT3536 target: PCR amplification was done with oJaL 188 and oAT967: 5′ GCGAACACGAACCCTAC 3′ (SEQ ID NO: 153) resulting in an amplicon on 2033 bp.

For pAT3537 target: PCR amplification was done with oJaL 188 and oAT3618: 5′ TCAAA-GCAGCAAACTCC 3′ (SEQ ID NO: 154) resulting in an amplicon on 2312 bp.

The amplicon was then gel purified by QIAquick Gel Extraction Kit (Qiagen) and sequenced using the primers for the PCR amplification to determine the mutation patterns at targeted region. All isolated green spore colonies showed no mutation at the target region or any of the sequenced amplicon. For white spore colonies from pAT3532, pAT3533, pAT3534, and pAT3535, none of them had mutations at the target site or in the rest of the amplicon. With respect to white spore colonies from pAT3536, one colony had the expected C17 mutation leading to a stop codon, and another colony had a silent mutation at C8 indicating that another mutation had happened elsewhere in the wA gene. With respect to white spore colonies from pAT3537, two colonies had the same expected C15 mutation leading to a stop codon.

The examples generated in A. oryzae supports the findings in A. niger and suggests that the primary target window of Mad7d-AID-UGI is C8 to C17, and that the Mad7d-AID-UGI system also can introduce C to T mutations in A. oryzae.

PART C: Bacillus licheniformis

### Example 9: Construction of a plasmid for expression of MAD7d-AID-UGI in Bacillus licheniformis

Plasmid pMDT452 was constructed for expression of MAD7d-AID-UGI in Bacillus. Plasmid pMDT452 comprises the MAD7d-AID-UGI coding sequence flanked upstream by promoter PamyL4199 (U.S. Pat. No. 6,100,063) and downstream by the aprH transcription terminator of Bacillus clausii in mobilizable plasmid vector pBC16 (Bernhard et al. (1978): Bacteriocin and Antibiotic Resistance Plasmids in Bacillus cereus and Bacillus subtilis. J. Bacteriol. 133, 897-903). Plasmid pMDT452 was introduced into conjugation donor strain Bacillus subtilis pp 3724 by transformation, resulting in strain pp 3724/pMDT452.

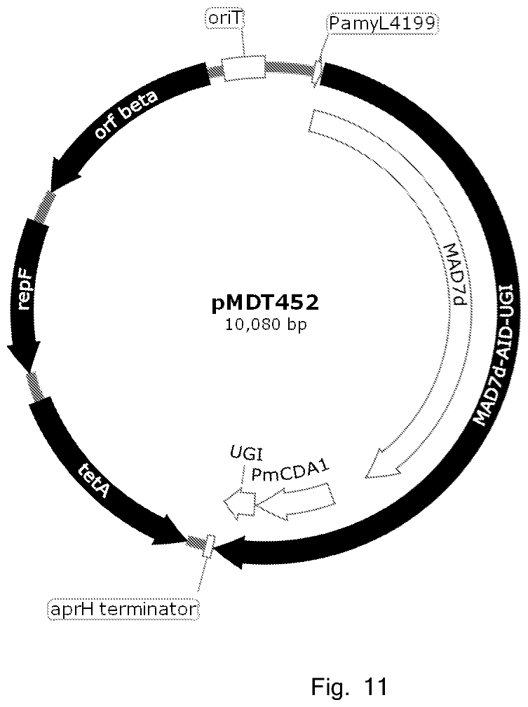

A map of pMDT452 is shown in FIG. 11, the DNA sequence of the MAD7d-AID-UGI coding region is shown in SEQ ID NO: 158, and the corresponding amino acid sequence is shown in SEQ ID NO: 159.

### Example 10. Construction of plasmids for expression of sgRNAs targeting a DsRED gene in Bacillus licheniformis

Plasmids pMDT454 and pMDT455 were constructed for expression of sgRNAs targeting the DsRED gene of Bacillus licheniformis strain MDT545. Each plasmid comprises an sgRNA cassette with the sgRNA expressed from the PamyQsc promoter (Prshort “consensus” amyQ; U.S. Pat. No. 6,255,076) in a plasmid vector based on temperature sensitive pAMB1-derived plasmid pWT (Bidnenko et al. (1998): In vivo relations between pAMB1-encoded type I topoisomerase and plasmid replication. Mol. Microbiol. 28, 1005-1016) and comprising the origin of transfer oriT of plasmid pUB110 (Selinger, L. B., McGregor, N. F., Khachatourians, G. G. and Hynes, M. F. (1990). Mobilization of closely related plasmids pUB110 and pBC16 by Bacillus plasmid pXO503 requires trans-acting open reading frame B. J. Bacteriol., 172, 3290-3297) for mobilization by conjugation. Two protospacers with different target positions within the DsRED coding sequence were designed (Table 16), each of which could form a premature stop codon when a C to T mutation is introduced by MAD7d-AID. Plasmids pMDT454 and pMDT455 were introduced into conjugation donor strain Bacillus subtilis pp 3724 by transformation, resulting in strains pp 3724/pMDT454 and pp 3724/pMDT455 respectively.

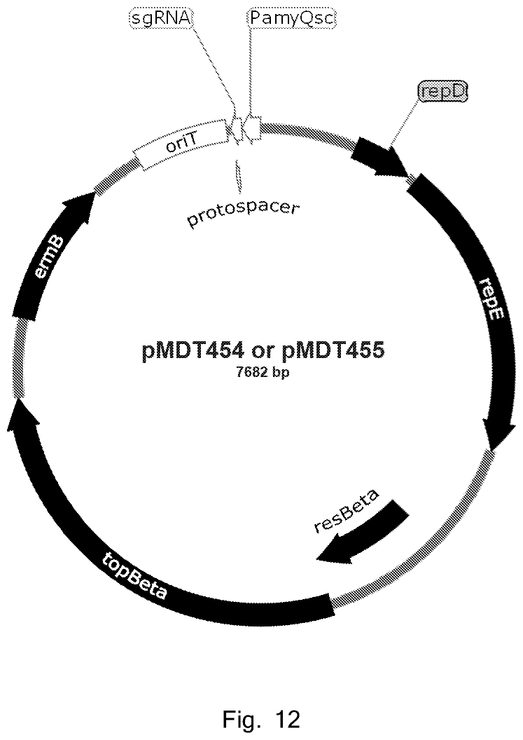

A map representing the structure of sgRNA plasmids pMDT454 and pMDT455 is shown in FIG. 12. As an example of the sgRNA plasmids, the DNA sequence of pMDT454 is shown in SEQ ID NO: 160.

TABLE 16
Protospacer sequences for disruption of DsRED
gene. Potential target C's are underlined.

| Plasmid | | Protospacer |
|---|---|---|
| (proto- | Protospacer (5′ → 3′), | adjacent motif |
| spacer | potential target C's | (PAM) sequence |
| number) | are underlined | (5′ → 3′) |
| pMDT454 | AGTCCGCAGTTTCAGTATGGG | CTTA |
| (PS1-AID) | (SEQ ID NO: 161) | |
| | sense strand | |
| pMDT455 | AGATGTCCCAAGCAAACGGCA | CTTA |
| (PS2-AID) | (SEQ ID NO: 162) | |
| | antisense strand | |

### Example 11. Introduction of MAD7d-AID-UGI plasmid and sgRNA plasmids for DsRED gene inactivation into Bacillus licheniformis

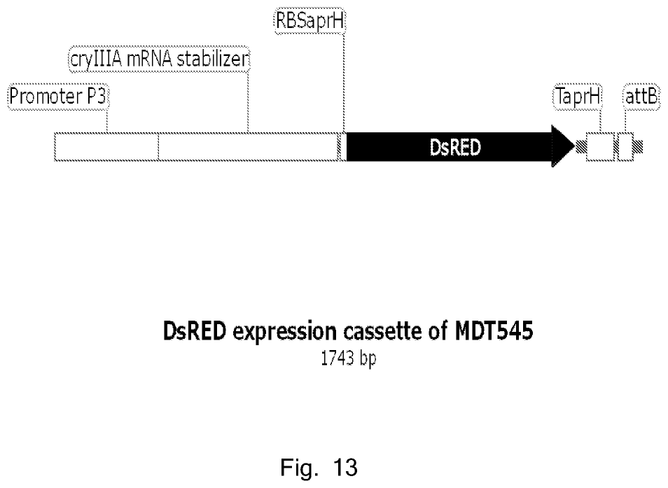

Bacillus licheniformis MDT545 is a derivative of Bacillus licheniformis SJ1904 comprising a DsRED expression cassette inserted at the amyL locus of the chromosome and a GFP expression cassette inserted at the xylA locus of the chromosome. A map of the inserted DsRED expression cassette is shown in FIG. 13, and the corresponding DNA sequence is shown in SEQ ID NO: 163; the DNA sequence of the DsRED coding region is shown in SEQ ID NO: 164.

Using conjugation donor strain pp 3724/pMDT452, plasmid pMDT452 was introduced by conjugation into Bacillus licheniformis MDT545, selecting for resistance to erythromycin at 34° C. The resulting strain was designated MDT545/pMDT452.

Using conjugation donor strains pp 3724/pMDT454 and pp 3724/pMDT455, sgRNA plasmids pMDT454 and pMDT455 were respectively introduced by conjugation into Bacillus licheniformis MDT545/pMDT452, selecting for resistance to both erythromycin and tetracycline at 34° C.

Colonies of strain MDT545 appear red, due to expression of DsRED. Disruption of the DsRED results in a strain that appears green, due to expression of GFP. However, binding of MAD7d to the DsRED gene can also result in gene silencing, resulting in reduction of DsRED expression and a green appearance of colonies. Colonies that appear green due to silencing of the DsRED gene will appear red again if either of the plasmids is lost, whereas colonies that appear green due to mutation of the DsRED gene will remain green even if one of the plasmids is lost. Therefore, in order to distinguish colonies in which the DsRED gene has been mutated from those in which the DsRED gene has merely been silenced, transconjugants must be re-moved from antibiotic selection in order to allow one or both of the plasmids to be lost.

After 2 days' incubation at 34° C., the selective plates from the sgRNA plasmid conjugations were flooded with 2 ml LB broth, and a sterile spreader was used to suspend transconjugant colonies into the broth. Fresh LB broth supplemented with 2 μg/ml erythromycin and 15 μg/ml tetracycline was then inoculated with the resulting cell suspension from each conjugation and incubated at 34° C. with shaking at 250 rpm. After overnight incubation, fresh LB broth without antibiotic was inoculated with each culture and incubated at 50° C. with shaking at 250 rpm to promote loss of the temperature-sensitive sgRNA plasmids.

After overnight incubation, dilutions of the LB cultures were plated on LB agar and incubated at 34° C. Green colonies were picked, and the DsRED coding region was amplified by PCR from 16 green colonies per conjugation. The PCRs were treated with the ExoSAP-IT PCR Product Cleanup kit (Applied Biosystems) and sequenced to determine the mutation patterns at the targeted region. As can be seen in Table 17, sequencing results indicated that one of the expected nonsense mutations had occurred in the targeted protospacer region of each green strain, which confirms that the nucleobase editing complex of the invention also works efficiently in bacterial cells. The examples generated in B. licheniformis supports the findings in A. oryzae and A. niger and suggests that the Mad7d-AID-UGI system also can introduce C to T mutations in B. licheniformis.

TABLE 17
Summary of results of dsRED-targeting MAD7d-
AID-UGI B. licheniformis conjugations

| | | Approximate | | Mutations |
|---|---|---|---|---|
| sgRNA | | frequency of green | Expected | observed in green |
| plasmid | Protospacer | colonies | mutation(s) | transconjugants |
| pMDT454 | PS1-AID | 0.5% | C190T (Q64*) or | C190T in 15 of 16; |
| | | | C196T (Q66*) | C196T in 1 of 16; |
| pMDT455 | PS2-AID | 1% | G173A or G174A | G173A in 6 of 16; |
| | | | (W58*) | G174A in 10 of 16; |

### List of Embodiments

1. A nucleobase editing complex comprising, consisting essentially of, or consisting of:
   a) a catalytically inactive RNA-guided endonuclease having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126 or SEQ ID NO: 155; and
   b) a nucleobase editing domain.
2. The nucleobase editing complex according to embodiment 1, wherein the catalytically inactive RNA-guided endonuclease comprises an alteration of an amino acid at a position corresponding to position 877 of SEQ ID NO: 126; preferably the amino acid at a position corresponding to position 877 of SEQ ID NO: 126 is substituted with Ala, Arg, Asn, Asp, Cys, Gln, Glu, Gly, His, 11e , Leu, Lys, Met, Phe, Pro, Ser, Thr, Trp, Tyr, or Val; most preferably the amino acid at a position corresponding to position 877 of SEQ ID NO: 126 is substituted with Ala.
3. The nucleobase editing domain according to any of the preceding embodiments, wherein the catalytically inactive RNA-guided endonuclease comprises, consists essentially of, or consists of SEQ ID NO: 126 or SEQ ID NO: 155.
4. The nucleobase editing complex according to any of the preceding embodiments, wherein the nucleobase editing domain is a cytosine base editor (CBE).
5. The nucleobase editing complex according to embodiment 4, wherein the nucleobase editing domain is a cytosine base editor of the APOBEC1/AID family; preferably the nucleobase editing domain is APOBEC1 or CDA1, in particular PmCDA1.
6. The nucleobase editing complex according to embodiment 4, wherein the nucleobase editing domain comprises or consists of a polypeptide having a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 128; preferably the nuclease editing domain comprises, consists essentially of, or consists of SEQ ID NO: 128.
7. The nucleobase editing complex according to any of embodiments 4-6, which further comprises an uracil DNA glycosylase inhibitor (UGI). Preferably, the uracil DNA glycosylase inhibitor has a sequence identity of at least 80%, such as e.g., at least 85%, at least 90%, at least 91%, at least 92%, at least 93%, at least 94%, at least 95%, at least 96%, at least 97%, at least 98%, at least 99%, or 100%, to SEQ ID NO: 132. Most preferably, the uracil DNA glycosylase inhibitor comprises, consists essentially of, or consists of SEQ ID NO: 132.
8. The nucleobase editing complex according to any of embodiments 1-2, wherein the nucleobase editing domain is an adenine base editor (ABE).; preferably the nucleobase editing domain is selected from the group consisting of TadA, TadA*, TadA homodimer, and TadA-TadA* heterodimer; most preferably the nucleobase editing domain is a TadA-TadA* heterodimer.
9. The nucleobase editing complex according to any of the preceding embodiments, wherein the catalytically inactive RNA-guided endonuclease and the nucleobase editing domain are fused end-to-end or connected via a linker polypeptide; preferably the catalytically inactive RNA-guided endonuclease, the linker polypeptide, and the nucleobase editing domain are encoded in frame and are expressed as a single polypeptide.
10. The nucleobase editing complex according to embodiment 9, wherein the linker polypeptide comprises at least 10 amino acid residues; preferably the linker polypeptide comprises at least 50 amino acid residues; most preferably the linker polypeptide comprises at least 100 amino acid residues.
11. The nucleobase editing complex according to any of embodiments 10-11, wherein the linker polypeptide has a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 130; preferably the linker polypeptide comprises, consists essentially of, or consists of SEQ ID NO: 130.
12. A polynucleotide encoding a nucleobase editing complex comprising, consisting essentially of, or consisting of:
   a) a catalytically inactive RNA-guided endonuclease having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126 or SEQ ID NO: 155; and
   b) a nucleobase editing domain.
13. The polynucleotide according to embodiment 12, wherein the catalytically inactive RNA-guided endonuclease and the nucleobase editing domain are encoded in frame and are expressed as a single polypeptide.
14. The polynucleotide of according to any of embodiments 12-13, which further comprises a third polynucleotide encoding a linker polypeptide, wherein the third polynucleotide is positioned between the 3′ end of the first polynucleotide and the 5′ end of the second polynucleotide, and wherein the first polynucleotide, the second polypeptide, and the third polypeptide are encoded in frame and expressed as a single polypeptide.
15. The polynucleotide according to any of embodiments 12-14, wherein the catalytically inactive RNA-guided endonuclease comprises an alteration of an amino acid at a position corresponding to position 877 of SEQ ID NO: 126; preferably the amino acid at a position corresponding to position 877 of SEQ ID NO: 126 is substituted with Ala, Arg, Asn, Asp, Cys, Gln, Glu, Gly, His, 11e , Leu, Lys, Met, Phe, Pro, Ser, Thr, Trp, Tyr, or Val; most preferably the amino acid at a position corresponding to position 877 of SEQ ID NO: 126 is substituted with Ala.
16. The polynucleotide according to any of embodiments 12-15, wherein the catalytically inactive RNA-guided endonuclease comprises, consists essentially of, or consists of SEQ ID NO: 126 or SEQ ID NO: 155.
17. The polynucleotide according to any of embodiments 12-16, wherein the nucleobase editing domain is a cytosine base editor (CBE).
18. The polynucleotide according to embodiment 17, wherein the nucleobase editing domain is a cytosine base editor of the APOBEC1/AID family; preferably the nucleobase editing domain is APOBEC1 or CDA1, in particular PmCDA1.
19. The polynucleotide according to embodiment 17, wherein the nucleobase editing domain comprises or consists of a polypeptide having a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 128; preferably the nuclease editing domain comprises, consists essentially of, or consists of SEQ ID NO: 128.
20. The polynucleotide according to any of embodiment 12-19, which further comprises an uracil DNA glycosylase inhibitor (UGI). Preferably, the uracil DNA glycosylase inhibitor has a sequence identity of at least 80%, such as e.g., at least 85%, at least 90%, at least 91%, at least 92%, at least 93%, at least 94%, at least 95%, at least 96%, at least 97%, at least 98%, at least 99%, or 100%, to SEQ ID NO: 132. Most preferably, the uracil DNA glycosylase inhibitor comprises, consists essentially of, or consists of SEQ ID NO: 132.
21. The polynucleotide according to any of embodiments 12-16, wherein the nucleobase editing domain is an adenine base editor (ABE).; preferably the nucleobase editing domain is selected from the group consisting of TadA, TadA*, TadA homodimer, and TadA-TadA* heterodimer; most preferably the nucleobase editing domain is a TadA-TadA* heterodimer.
22. The polynucleotide according to any of embodiments 14-21, wherein the linker polypeptide comprises at least 10 amino acid residues; preferably the linker polypeptide comprises at least 50 amino acid residues; most preferably the linker polypeptide comprises at least 100 amino acid residues.
23. The polynucleotide according to any of embodiments 14-22, wherein the linker polypeptide has a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 130; preferably the linker polypeptide comprises, consists essentially of, or consists of SEQ ID NO: 130.
24. A nucleic acid construct comprising a polynucleotide encoding a nucleobase editing complex comprising, consisting essentially of, or consisting of:
   a) a catalytically inactive RNA-guided endonuclease having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126 or SEQ ID NO: 155; and
   b) a nucleobase editing domain.
25. The nucleic acid construct according to embodiment 24, wherein the catalytically inactive RNA-guided endonuclease and the nucleobase editing domain are encoded in frame and are expressed as a single polypeptide.
26. The nucleic acid construct according to any of embodiments 24-25, which further comprises a third polynucleotide encoding a linker polypeptide, wherein the third polynucleotide is positioned between the 3′ end of the first polynucleotide and the 5′ end of the second polynucleotide, and wherein the first polynucleotide, the second polypeptide, and the third polypeptide are encoded in frame and expressed as a single polypeptide.
27. The nucleic acid construct according to any of embodiments 24-26, wherein the catalytically inactive RNA-guided endonuclease comprises an alteration of an amino acid at a position corresponding to position 877 of SEQ ID NO: 126; preferably the amino acid at a position corresponding to position 877 of SEQ ID NO: 126 is substituted with Ala, Arg, Asn, Asp, Cys, Gln, Glu, Gly, His, 11e , Leu, Lys, Met, Phe, Pro, Ser, Thr, Trp, Tyr, or Val; most preferably the amino acid at a position corresponding to position 877 of SEQ ID NO: 126 is substituted with Ala.
28. The nucleic acid construct according to any of embodiments 24-27, wherein the catalytically inactive RNA-guided endonuclease comprises, consists essentially of, or consists of SEQ ID NO: 126 or SEQ ID NO: 155.
29. The nucleic acid construct according to any of embodiments 24-28, wherein the nucleobase editing domain is a cytosine base editor (CBE).
30. The nucleic acid construct according to embodiment 29, wherein the nucleobase editing domain is a cytosine base editor of the APOBEC1/AID family; preferably the nucleobase editing domain is APOBEC1 or CDA1, in particular PmCDA1.
31. The nucleic acid construct according to embodiment 29-30, wherein the nucleobase editing domain comprises or consists of a polypeptide having a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 128; preferably the nuclease editing domain comprises, consists essentially of, or consists of SEQ ID NO: 128.
32. The nucleic acid construct according to any of embodiment 24-31, which further comprises an uracil DNA glycosylase inhibitor (UGI). Preferably, the uracil DNA glycosylase inhibitor has a sequence identity of at least 80%, such as e.g., at least 85%, at least 90%, at least 91%, at least 99%, or 100%, to SEQ ID NO: 132. Most preferably, the uracil DNA glycosylase inhibitor comprises, consists essentially of, or consists of SEQ ID NO: 132.
33. The nucleic acid construct according to any of embodiments 24-28, wherein the nucleobase editing domain is an adenine base editor (ABE).; preferably the nucleobase editing domain is selected from the group consisting of TadA, TadA*, TadA homodimer, and TadA-TadA* heterodimer; most preferably the nucleobase editing domain is a TadA-TadA* heterodimer.
34. The nucleic acid construct according to any of embodiments 26-33, wherein the linker polypeptide comprises at least 10 amino acid residues; preferably the linker polypeptide comprises at least 50 amino acid residues; most preferably the linker polypeptide comprises at least 100 amino acid residues.
35. The nucleic acid construct according to any of embodiments 26-34, wherein the linker polypeptide has a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 130; preferably the linker polypeptide comprises, consists essentially of, or consists of SEQ ID NO: 130.
36. An expression vector comprising:
   I) a polynucleotide encoding a nucleobase editing complex comprising, consisting essentially of, or consisting of:
   a) a catalytically inactive RNA-guided endonuclease having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126 or SEQ ID NO: 155; and
   b) a nucleobase editing domain; OR
   II) a nucleic acid construct comprising a polynucleotide encoding a nucleobase editing complex comprising, consisting essentially of, or consisting of:
   a) a catalytically inactive RNA-guided endonuclease having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126 or SEQ ID NO: 155; and
   b) a nucleobase editing domain.
37. The expression vector according to embodiment 36, wherein the catalytically inactive RNA-guided endonuclease and the nucleobase editing domain are encoded in frame and are expressed as a single polypeptide.
38. The expression vector according to any of embodiments 36-37, which further comprises a third polynucleotide encoding a linker polypeptide, wherein the third polynucleotide is positioned between the 3′ end of the first polynucleotide and the 5′ end of the second polynucleotide, and wherein the first polynucleotide, the second polypeptide, and the third polypeptide are encoded in frame and expressed as a single polypeptide.
39. The expression vector according to any of embodiments 36-38, wherein the catalytically inactive RNA-guided endonuclease comprises an alteration of an amino acid at a position corresponding to position 877 of SEQ ID NO: 126; preferably the amino acid at a position corresponding to position 877 of SEQ ID NO: 126 is substituted with Ala, Arg, Asn, Asp, Cys, Gln, Glu, Gly, His, 11e , Leu, Lys, Met, Phe, Pro, Ser, Thr, Trp, Tyr, or Val; most preferably the amino acid at a position corresponding to position 877 of SEQ ID NO: 126 is substituted with Ala.
40. The expression vector according to any of embodiments 36-39, wherein the catalytically inactive RNA-guided endonuclease comprises, consists essentially of, or consists of SEQ ID NO: 126 or SEQ ID NO: 155.
41. The expression vector according to any of embodiments 36-40, wherein the nucleobase editing domain is a cytosine base editor (CBE).
42. The expression vector according to embodiment 41, wherein the nucleobase editing domain is a cytosine base editor of the APOBEC1/AID family; preferably the nucleobase editing domain is APOBEC1 or CDA1, in particular PmCDA1.
43. The expression vector according to embodiment 41-42, wherein the nucleobase editing domain comprises or consists of a polypeptide having a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 128; preferably the nuclease editing domain comprises, consists essentially of, or consists of SEQ ID NO: 128.
44. The expression vector according to any of embodiment 36-43, which further comprises an uracil DNA glycosylase inhibitor (UGI). Preferably, the uracil DNA glycosylase inhibitor has a sequence identity of at least 80%, such as e.g., at least 85%, at least 90%, at least 91%, at least 99%, or 100%, to SEQ ID NO: 132. Most preferably, the uracil DNA glycosylase inhibitor comprises, consists essentially of, or consists of SEQ ID NO: 132.
45. The expression vector according to any of embodiments 36-40, wherein the nucleobase editing domain is an adenine base editor (ABE).; preferably the nucleobase editing domain is selected from the group consisting of TadA, TadA*, TadA homodimer, and TadA-TadA* heterodimer; most preferably the nucleobase editing domain is a TadA-TadA* heterodimer.
46. The expression vector according to any of embodiments 38-45, wherein the linker polypeptide comprises at least 10 amino acid residues; preferably the linker polypeptide comprises at least 50 amino acid residues; most preferably the linker polypeptide comprises at least 100 amino acid residues.
47. The expression vector according to any of embodiments 38-46, wherein the linker polypeptide has a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 130; preferably the linker polypeptide comprises, consists essentially of, or consists of SEQ ID NO: 130.
48. A host cell comprising:
   I) a nucleobase editing complex comprising, consisting essentially of, or consisting of:
   a) a catalytically inactive RNA-guided endonuclease having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126 or SEQ ID NO: 155; and
   b) a nucleobase editing domain;
   II) a polynucleotide encoding a nucleobase editing complex comprising, consisting essentially of, or consisting of:
   a) a catalytically inactive RNA-guided endonuclease having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126 or SEQ ID NO: 155; and
   b) a nucleobase editing domain;
   III) a nucleic acid construct comprising a polynucleotide encoding a nucleobase editing complex comprising, consisting essentially of, or consisting of:
   a) a catalytically inactive RNA-guided endonuclease having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126 or SEQ ID NO: 155; and
   b) a nucleobase editing domain; AND/OR
   IV) an expression vector comprising:
   A) a polynucleotide encoding a nucleobase editing complex comprising, consisting essentially of, or consisting of:
   a) a catalytically inactive RNA-guided endonuclease having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126 or SEQ ID NO: 155; and
   b) a nucleobase editing domain; OR
   B) a nucleic acid construct comprising a polynucleotide encoding a nucleobase editing complex comprising, consisting essentially of, or consisting of:
   a) a catalytically inactive RNA-guided endonuclease having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126 or SEQ ID NO: 155; and
   b) a nucleobase editing domain.
49. The host cell of embodiment 48, which is a prokaryotic or eukaryotic host cell.
50. The host cell according to any of embodiments 48-49, which is a bacterial host cell; preferably the bacterial host cell is a Bacillus, Escherichia, Lactobacillus, Lactococcus Streptococcus, or Streptomyces cell; more preferably the bacterial host cell is selected from the group consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, Bacillus thuringiensis, Escherichia coli, Lactobacillus acidophilus, Lactobacillus amylovorus, Lactobacillus brevis, Lactobacillus (para) casei, Lactobacillus cellobiosus, Lactobacillus crispatus, Lactobacillus curvatus, Lactobacillus delbrueckii subsp. bulgaricus, L. delbrueckii subsp. lactis, Lactobacillus fermentum, Lactobacillus gallinarum, Lactobacillus gasseri, Lactobacillus helveticus, Lactobacillus johnsonii, Lactobacillus plantarum, Lactobacillus reuteri, Lactobacillus rhamnosus, Lactobacillus salivarius, Lactococcus chungangensis, Lactococcus formosensis, Lactococcus fujiensis, Lactococcus garvieae, Lactococcus lactis, Lactococcus piscium, Lactococcus plantarum, Lactococcus raffinolactis, Lactococcus taiwanensi, Streptococcus equisimilis, Streptococcus pyogenes, Streptococcus uberis, Streptococcus equi subsp. zooepidemicus, Streptomyces achromogenes, Streptomyces avermitilis, Streptomyces coelicolor, Streptomyces griseus, and Streptomyces lividans; most preferably the bacterial host cell is Bacillus licheniformis.
51. The host cell according to any of embodiments 48-49, which is a filamentous fungal host cell; preferably the fungal host cell is a Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell; more preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell; most preferably the filamentous fungal host cell is a Aspergillus niger, Aspergillus oryzae, or Trichoderma reesei cell.
52. The host cell according to any of embodiments 48-49, which is a yeast host cell; preferably the yeast host cell is a Candida, Hansenula, Kluyveromyces, Pichia, Saccharomyces, Schizosaccharomyces, and Yarrowia cell; more preferably the yeast host cell is selected from the group consisting of Kluyveromyces lactis, Pichia pastoris, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell; most preferably the yeast host cell is Pichia pastoris.
53. The host cell according to any of embodiments 48-49, which is a mammalian host cell; preferably the mammalian host cell is a mouse, rat, or human cell.
54. The host cell according to any of embodiments 48-53, wherein the catalytically inactive RNA-guided endonuclease and the nucleobase editing domain are encoded in frame and are expressed as a single polypeptide.
55. The host cell according to any of embodiments 48-54, wherein the catalytically inactive RNA-guided endonuclease and the nucleobase editing domain are fused end-to-end or connected via a linker polypeptide; preferably the catalytically inactive RNA-guided endonuclease, the linker polypeptide, and the nucleobase editing domain are encoded in frame and are expressed as a single polypeptide.
56. The host cell according to any of embodiments 48-55, wherein the catalytically inactive RNA-guided endonuclease comprises an alteration of an amino acid at a position corresponding to position 877 of SEQ ID NO: 126; preferably the amino acid at a position corresponding to position 877 of SEQ ID NO: 126 is substituted with Ala, Arg, Asn, Asp, Cys, Gln, Glu, Gly, His, Ile, Leu, Lys, Met, Phe, Pro, Ser, Thr, Trp, Tyr, or Val; most preferably the amino acid at a position corresponding to position 877 of SEQ ID NO: 126 is substituted with Ala.
57. The host cell according to any of embodiments 48-56, wherein the catalytically inactive RNA-guided endonuclease comprises, consists essentially of, or consists of SEQ ID NO: 126 or SEQ ID NO: 155.
58. The host cell according to any of embodiments 48-57, wherein the nucleobase editing domain is a cytosine base editor (CBE).
59. The host cell according to embodiment 58, wherein the nucleobase editing domain is a cytosine base editor of the APOBEC1/AID family; preferably the nucleobase editing domain is APOBEC1 or CDA1, in particular PmCDA1.
60. The host cell according to embodiment 58-59, wherein the nucleobase editing domain comprises or consists of a polypeptide having a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 128; preferably the nuclease editing domain comprises, consists essentially of, or consists of SEQ ID NO: 128.
61. The host cell according to any of embodiment 48-60, wherein the nucleobase editing complex further comprises an uracil DNA glycosylase inhibitor (UGI). Preferably, the uracil DNA glycosylase inhibitor has a sequence identity of at least 80%, such as e.g., at least 85%, at least 90%, at least 91%, at least 92%, at least 93%, at least 94%, at least 95%, at least 96%, at least 97%, at least 98%, at least 99%, or 100%, to SEQ ID NO: 132. Most preferably, the uracil DNA glycosylase inhibitor comprises, consists essentially of, or consists of SEQ ID NO: 132.
62. The host cell according to any of embodiments 48-57, wherein the nucleobase editing domain is an adenine base editor (ABE).; preferably the nucleobase editing domain is selected from the group consisting of TadA, TadA*, TadA homodimer, and TadA-TadA* heterodimer; most preferably the nucleobase editing domain is a TadA-TadA* heterodimer.
63. The host cell according to any of embodiments 55-62, wherein the linker polypeptide comprises at least 10 amino acid residues; preferably the linker polypeptide comprises at least 50 amino acid residues; most preferably the linker polypeptide comprises at least 100 amino acid residues.
64. The host cell according to any of embodiments 55-63, wherein the linker polypeptide has a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 130; preferably the linker polypeptide comprises, consists essentially of, or consists of SEQ ID NO: 130.
65. A method for modifying at least one nucleobase in a DNA target sequence, the method comprising:
   I) providing a nucleobase editing complex comprising, consisting essentially of, or consisting of:
   a) a catalytically inactive RNA-guided endonuclease having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO: 126 or SEQ ID NO: 155; and
   b) a nucleobase editing domain;
   wherein the nucleobase editing complex is complexed with a gRNA that is complementary to and capable of hybridizing to the DNA target sequence; and
   II) contacting the nucleobase editing complex with the DNA target sequence;
   wherein at least one nucleobase in the DNA target sequence is converted to a different nucleobase without introducing a double-strand break in the DNA sequence of interest.
66. The method according to embodiment 65, wherein the catalytically inactive RNA-guided endonuclease comprises an alteration of an amino acid at a position corresponding to position 877 of SEQ ID NO: 126; preferably the amino acid at a position corresponding to position 877 of SEQ ID NO: 126 is substituted with Ala, Arg, Asn, Asp, Cys, Gln, Glu, Gly, His, 11e , Leu, Lys, Met, Phe, Pro, Ser, Thr, Trp, Tyr, or Val; most preferably the amino acid at a position corresponding to position 877 of SEQ ID NO: 126 is substituted with Ala.
67. The method according to any of embodiments 65-66, wherein the catalytically inactive RNA-guided endonuclease comprises, consists essentially of, or consists of SEQ ID NO: 126 or SEQ ID NO: 155.
68. The method according to any of embodiments 65-67, wherein the nucleobase editing domain is a cytosine base editor (CBE).
69. The method according to embodiment 68, wherein the nucleobase editing domain is a cytosine base editor of the APOBEC1/AID family; preferably the nucleobase editing domain is APOBEC1 or CDA1, in particular PmCDA1.
70. The method according to embodiment 68-69, wherein the nucleobase editing domain comprises or consists of a polypeptide having a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 128; preferably the nuclease editing domain comprises, consists essentially of, or consists of SEQ ID NO: 128.
71. The method according to any of embodiments 68-70, which further comprises an uracil DNA glycosylase inhibitor (UGI). Preferably, the uracil DNA glycosylase inhibitor has a sequence identity of at least 80%, such as e.g., at least 85%, at least 90%, at least 91%, at least 92%, at least 93%, at least 94%, at least 95%, at least 96%, at least 97%, at least 98%, at least 99%, or 100%, to SEQ ID NO: 132. Most preferably, the uracil DNA glycosylase inhibitor comprises, consists essentially of, or consists of SEQ ID NO: 132.
72. The method according to any of embodiments 65-67, wherein the nucleobase editing domain is an adenine base editor (ABE).; preferably the nucleobase editing domain is selected from the group consisting of TadA, TadA*, TadA homodimer, and TadA-TadA* heterodimer; most preferably the nucleobase editing domain is a TadA-TadA* heterodimer.
73. The method according to any of embodiments 65-72, wherein the catalytically inactive RNA-guided endonuclease and the nucleobase editing domain are fused end-to-end or connected via a linker polypeptide; preferably the catalytically inactive RNA-guided endonuclease, the linker polypeptide, and the nucleobase editing domain are encoded in frame and are expressed as a single polypeptide.
74. The method according to embodiment 73, wherein the linker polypeptide comprises at least 10 amino acid residues; preferably the linker polypeptide comprises at least 50 amino acid residues; most preferably the linker polypeptide comprises at least 100 amino acid residues.
75. The method according to any of embodiments 73-74, wherein the linker polypeptide has a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 130; preferably the linker polypeptide comprises, consists essentially of, or consists of SEQ ID NO: 130.
76. The method according to any of embodiments 65-75, wherein the DNA target sequence comprises a polynucleotide comprising 21 nucleotides that are at least 85% complementary to and capable of hybridizing to the gRNA; preferably the 21 nucleotides are at least 90%, 95%, 97%, 98%, 99% or even 100% complementary to and capable of hybridizing to the gRNA.
77. The method according to any of embodiments 65-76, wherein the DNA target sequence is flanked by a functional protospacer adjacent motif (PAM) that is recognized by the catalytically inactive RNA-guided endonuclease; preferably the PAM sequence is 5′-TTTN-3′ or 5′-CTTN-3′; more preferably the PAM sequence is 5′-TTTN-3′; most preferably the PAM sequence is 5′-TTTC-3′ or 5′-TTTG-3′.
78. The method according to embodiment 77, wherein the DNA target sequence is located at the 3′ end of the PAM sequence; preferably the DNA target sequence is located directly adjacent to the 3′ end of the PAM sequence.
79. The method according to any of embodiments 65-78, wherein the DNA target sequence is comprised in an open reading frame encoding a polypeptide or in a promoter region; preferably the DNA target sequence encode one or more enzyme selected from the group consisting of hydrolase, isomerase, ligase, lyase, oxidoreductase, or a transferase; preferably the one or more enzyme is an alpha-amylase, alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucano-transferase, glucoamylase, invertase, laccase, lipase, mannosidase, mutanase, oxidase, pectinolytic enzyme, peroxidase, phosphodiesterase, phytase, polyphenoloxidase, proteolytic enzyme, ribonuclease, transglutaminase, and xylanase.

1-21. (canceled)

22. A nucleobase editing complex comprising:
a) a catalytically inactive RNA-guided endonuclease having a sequence identity of at least 60% to SEQ ID NO: 126; and
b) a nucleobase editing domain.

23. The nucleobase editing complex according to claim 22, wherein the catalytically inactive RNA-guided endonuclease comprises an alteration of an amino acid at a position corresponding to position 877 of SEQ ID NO: 126.

24. The nucleobase editing complex according to claim 22, wherein the amino acid at a position corresponding to position 877 of SEQ ID NO: 126 is substituted with Ala.

25. The nucleobase editing domain according to claim 22, wherein the catalytically inactive RNA-guided endonuclease comprises SEQ ID NO: 126.

26. The nucleobase editing complex according to claim 22, wherein the nucleobase editing domain is a cytosine base editor (CBE).

27. The nucleobase editing complex according to claim 26, wherein the nucleobase editing domain is a cytosine base editor of the APOBEC1/AID family.

28. The nucleobase editing complex according to claim 26, wherein the nucleobase editing domain is PmCDA1.

29. The nucleobase editing complex according to claim 26, wherein the nucleobase editing domain comprises or consists of a polypeptide having a sequence identity of at least 80% to SEQ ID NO: 128.

30. The nucleobase editing complex according to claim 26, wherein the nuclease editing domain comprises SEQ ID NO: 128.

31. The nucleobase editing complex according to claim 26, which further comprises an uracil DNA glycosylase inhibitor (UGI).

32. The nucleobase editing complex according to claim 31, wherein the uracil DNA glycosylase inhibitor has a sequence identity of at least 80% to SEQ ID NO: 132.

33. The nucleobase editing complex according to claim 31, the uracil DNA glycosylase inhibitor comprises SEQ ID NO: 132.

34. The nucleobase editing complex according to claim 22, wherein the nucleobase editing domain is an adenine base editor (ABE).

35. The nucleobase editing complex according to claim 22, wherein the nucleobase editing domain is selected from the group consisting of TadA, TadA*, TadA homodimer, and TadA-TadA* heterodimer.

36. The nucleobase editing complex according to claim 22, wherein the nucleobase editing domain is a TadA-TadA* heterodimer.

37. The nucleobase editing complex according to claim 22, wherein the catalytically inactive RNA-guided endonuclease and the nucleobase editing domain are fused end-to-end or connected via a linker polypeptide.

38. The nucleobase editing complex according to claim 22, wherein the catalytically inactive RNA-guided endonuclease, the linker polypeptide, and the nucleobase editing domain are encoded in frame and are expressed as a single polypeptide.

39. The nucleobase editing complex according to claim 38, wherein the linker polypeptide comprises at least 10 amino acid residues.

40. The nucleobase editing complex according to claim 38, wherein the linker polypeptide comprises at least 50 amino acid residues

41. The nucleobase editing complex according to claim 38, wherein the linker polypeptide has a sequence identity of at least 80% to SEQ ID NO: 130.

42. The nucleobase editing complex according to claim 38, wherein the linker polypeptide comprises SEQ ID NO: 130.

43. A polynucleotide encoding a nucleobase editing complex according to claim 22.

44. A host cell comprising the polynucleotide according to claim 43.

45. The host cell according to claim 44, which is a bacterial host cell.

46. The host cell according to claim 45, wherein the bacterial host cell is Bacillus licheniformis.

47. The host cell according to claim 44, which is a filamentous fungal host cell.

48. The host cell according to claim 47, wherein the filamentous fungal host cell is a Aspergillus niger, Aspergillus oryzae, or Trichoderma reesei cell.

49. The host cell according to claim 44, which is a yeast host cell.

50. The host cell according to claim 49, wherein the yeast host cell is Pichia pastoris.

51. The host cell according to claim 44, which is a mammalian host cell.

52. A method for modifying at least one nucleobase in a DNA target sequence, the method comprising:
a) providing a nucleobase editing complex according to claim 22 complexed with a gRNA that is complementary to and capable of hybridizing to the DNA target sequence; and
b) contacting the nucleobase editing complex with the DNA target sequence;
wherein at least one nucleobase in the DNA target sequence is converted to a different nucleobase without introducing a double-strand break in the DNA sequence of interest.
